(19) United States
(12) Patent Application Publication
(10) Pub. No.: US 2025/0171738 A1
(43) Pub. Date: May 29, 2025

(54) PROCESS FOR PRODUCING CONE PHOTORECEPTOR CELLS

(71) Applicant: CENTRE FOR EYE RESEARCH AUSTRALIA LTD, East Melbourne (AU)

(72) Inventors: Ching Bong WONG, East Melbourne (AU); Daniel URRUTIA-CABRERA, East Melbourne (AU)

(21) Appl. No.: 18/721,139

(22) PCT Filed: Dec. 22, 2022
(86) PCT No.: PCT/AU2022/051572

§ 371 Date: Jun. 17, 2024

(30) Foreign Application Priority Data
Dec. 22, 2021 (AU) 2021904199

(51) Int. Cl. C12N 5/0793; A61K 35/30; A61P 27/02; C07K 14/47; C12N 15/86

(52) U.S. Cl. CPC C12N 5/062; A61K 35/30; A61P 27/02; C07K 14/4702; C12N 15/86; C12N 2506/08; C12N 2510/00; C12N 2750/14143; C12N 2830/48; C12N 2830/50

(57) ABSTRACT

The invention relates to methods and compositions for the in vitro or in vivo converting one cell type to another cell type. Specifically, the invention relates to transdifferentiation of a cell to a cone photoreceptor cell. In one aspect, the invention provides a method for reprogramming a source cell, the method comprising increasing the protein expression of one or more transcription factors, or biologically active fragments or variants thereof, in the source cell, wherein the source cell is reprogrammed to exhibit at least one characteristic of a target cell, wherein the source cell is a glial cell, the target cell is a cone photoreceptor cell or cone-like photoreceptor cell; and the transcription factors are one or more of those selected from NEUROG2, CRX, RAX, RORA, NEUROD1, OTX2, ASCL1, PAX6, THRB, MEF2C, FOXP1 and ONECUT1.

### FIELD OF THE INVENTION

The invention relates to methods and compositions for the in vitro or in vivo converting of one cell type to another cell type. Specifically, the invention relates to transdifferentiation of a cell to a cone photoreceptor cell.

### RELATED APPLICATION

This application claims priority from Australian provisional application 2021904199, the entire contents of which are hereby incorporated by reference in their entirety.

### BACKGROUND OF THE INVENTION

Cell-based regenerative therapy requires the generation of specific cell types for replacing tissues damaged by injury, disease or age. Embryonic stem cells (ESC) have the potential to differentiate in every cell type from the (human) body and have therefore been extensively studied as a source for replacement therapy. However, ESC cannot be derived in a patient-specific fashion since they are established from cultured blastocysts. Therefore, immune rejection and ethical concerns are the main barriers that prevent the transfer of the ESC technology, and in particular of human ESC technology, to clinical applications.

Cell-replacement therapies have the potential to rapidly generate a variety of therapeutically important cell types directly from one's own easily accessible tissues, such as skin or blood. Such immunologically-matched cells would also pose less risk for rejection after transplantation. Moreover, these cells would manifest less tumorigenicity since they are terminally differentiated.

Trans-differentiation, the process of converting from one cell type to another without going through a pluripotent state, may have great promise for regenerative medicine but has yet to be reliably applied. Although it may be possible to switch the phenotype of one somatic cell type to another, the elements required for conversion are difficult to identify and in most instances unknown. The identification of factors to directly reprogram the identity of cell types is currently limited by, amongst other things, the cost of exhaustive experimental testing of plausible sets of factors, an approach that is inefficient and unscalable.

Photoreceptor cells, also known simply as photoreceptors, are light-sensing cells within the retina that form the basis of human vision. Cone cells, or cones, are photoreceptor cells in the retinas of vertebrate eyes including the human eye. They respond differently to light of different wavelengths, and are thus responsible for colour vision, and function best in relatively bright light, as opposed to rod cells, which work better in dim light.

The degeneration of photoreceptors is a central hallmark of many blinding diseases, including retinitis pigmentosa, age-related macular degeneration, choroideremia and diabetic retinopathy. These diseases affect millions worldwide and results in a significant socio-economic burden on our healthcare system. Critically, there is no cure to blindness once the photoreceptors in the eye are lost. Also, at the late stages of these retinal degenerative diseases, there are often insufficient remaining photoreceptors that can be targeted for pharmacological treatment. In this regard, regenerative medicine represents a highly attractive approach to address this issue.

There is a need for a new and/or improved method for generating cells and cell populations, particularly cone photoreceptor cells, for use in research and therapeutic applications.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention relates to in vitro, ex vivo or in vivo methods and compositions for direct reprogramming (i.e. transdifferentiation or cellular reprogramming) of a source cell to a cell having characteristics of a cone photoreceptor cell.

In one aspect, the present invention provides a method for reprogramming a source cell, the method comprising increasing the protein expression of one or more transcription factors, or biological active fragments or variants thereof, in the source cell, wherein the source cell is reprogrammed to exhibit at least one characteristic of a target cell, wherein:
the source cell is a glial cell,
the target cell is a cone photoreceptor cell or a cone-like photoreceptor cell; and
the transcription factors are one or more of those selected from NEUROG2, CRX, RAX, RORA, NEUROD1, OTX2, ASCL1, PAX6, THRB, MEF2C, FOXP1 and ONECUT1.

In one aspect, the present invention provides an in vitro, ex vivo, or in vivo method for reprogramming a source cell, the method comprising increasing the protein expression of one or more transcription factors, or biologically active fragments or variants thereof, in the source cell, wherein the source cell is reprogrammed to exhibit at least one characteristic of a target cell, wherein:
the source cell is a glial cell,
the target cell is a cone photoreceptor cell or a cone-like photoreceptor cell; and
the transcription factors are one or more of those selected from NEUROG2, CRX, RAX, RORA, NEUROD1, OTX2, ASCL1, PAX6, THRB, MEF2C, FOXP1 and ONECUT1.

Preferably, the glial cell is selected from the group consisting of a Müller glial (MG) cell, an astrocyte and a microglia. The glial cell may be a retinal glial cell.

The cone photoreceptor cell may be a L (or Long) type, M (or Medium) type, or S (or Short) type. The methods described herein may generate one or more of these types of cone photoreceptor cells.

In another aspect, the present invention provides a method of generating a cell exhibiting at least one characteristic of a cone photoreceptor cell from a source cell, the method comprising:
increasing the amount of one or more transcription factors, or biologically active fragments or variants thereof, in the source cell; and
culturing the source cell for a sufficient time and under conditions to allow differentiation to a cone photoreceptor cell; thereby generating the cell exhibiting at least one characteristic of a cone photoreceptor cell from a source cell, wherein:
the source cell is a glial cell, and
the transcription factors are one or more of those selected from NEUROG2, CRX, RAX, RORA, NEUROD1, OTX2, ASCL1, PAX6, THRB, MEF2C, FOXP1 and ONECUT1.

In any aspect or embodiment, the glial cell is selected from the group consisting a Müller glial (MG) cell, an astrocyte and a microglia. The glial cell may be a retinal glial cell.

In another aspect, the present invention provides a method for reprogramming a source cell to a cell that exhibits at least one characteristic of a cone photoreceptor cell, the method comprising:
providing a source cell, or a cell population comprising a source cell;
transfecting said source cell with one or more nucleic acids comprising a nucleotide sequence that encodes one or more transcription factors; and
culturing said cell or cell population, and optionally monitoring the cell or cell population for at least one characteristic of a cone photoreceptor cell, wherein:
the source cell is a glial cell, and
the transcription factors are one or more of those selected from NEUROG2, CRX, RAX, RORA, NEUROD1, OTX2, ASCL1, PAX6, THRB, MEF2C, FOXP1 and ONECUT1.

Further still, the present invention provides an in vitro, ex vivo or in vivo method for reprogramming a source cell to a cell that exhibits at least one characteristic of a cone photoreceptor cell, the method comprising:
providing a source cell, or a cell population comprising a source cell;
transfecting said source cell with one or more nucleic acids for increasing the expression of one or more genes encoding one or more transcription factors; and
culturing said cell or cell population, and optionally monitoring the cell or cell population for at least one characteristic of a cone photoreceptor cell, wherein:
the source cell is a glial cell and
the transcription factors are one or more of those selected from NEUROG2, CRX, RAX, RORA, NEUROD1, OTX2, ASCL1, PAX6, THRB, MEF2C, FOXP1 and ONECUT1.

Preferably, the one or more nucleic acids comprise sgRNAs for use in a CRISPR activation system for increasing the expression of the genes encoding the transcription factors. The sgRNA may be any sgRNA for increasing expression of one or more of NEUROG2, CRX, RAX, RORA, NEUROD1, OTX2, ASCL1, PAX6, THRB, MEF2C, FOXP1 and ONECUT1. The sgRNA may be one or more of those described herein.

In any aspect of the present invention, the glial cell is selected from the group consisting of a Müller glial (MG) cell, an astrocyte and a microglia.

In any aspect, the method comprises transfecting the source cell with nucleic acids encoding or for increasing the expression of least two of: NEUROG2, CRX, RAX, RORA, NEUROD1, OTX2, ASCL1, PAX6, THRB, MEF2C, FOXP1 and ONECUT1; at least three of NEUROG2, CRX, RAX, RORA, NEUROD1, OTX2, ASCL1, PAX6, THRB, MEF2C, FOXP1 and ONECUT1; at least four of NEUROG2, CRX, RAX, RORA, NEUROD1, OTX2, ASCL1, PAX6, THRB, MEF2C, FOXP1 and ONECUT1; at least five of NEUROG2, CRX, RAX, RORA, NEUROD1, OTX2, ASCL1, PAX6, THRB, MEF2C, FOXP1 and ONECUT1; at least six of NEUROG2, CRX, RAX, RORA, NEUROD1, OTX2, ASCL1, PAX6, THRB, MEF2C, FOXP1 and ONECUT1.

In any aspect of a method of the invention described herein, the source cell is a glial cell (e.g. Müller glial cell), and the transcription factors, or biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2;
(k) CRX, NEUROD1 and THRB;
(l) OTX2, RAX, and PAX6;
(m) ASCL1, NEUROD1, and OTX2;
(n) MEF2C, RAX and THRB;
(o) MEF2C, PAX6 and OTX2;
(p) MEF2C, OTX2 and THRB;
(q) MEF2C, OTX2 and RAX;
(r) ASCL1, CRX and FOXP1;
(s) CRX, NEUROG2, THRB and RAX;
(t) ASCL1, CRX, MEF2C, NEUROD1, OTX2 and THRB;
(u) MEF2C, PAX6 and THRB;
(v) MEF2C, NEUROD1 and PAX6;
(w) ASCL1, OTX2 and RAX;
(x) ASCL1, NEUROG2 and PAX6;
(y) ASCL1, CRX and RAX;
(z) CRX, NEUROD1 and OTX2;
(aa) CRX, NEUROG2 and OTX2;
(bb) ASCL1, CRX and NEUROG2;
(cc) CRX, RORA and THRB;
(dd) NEUROD1, OTX2 and RAX;
(ee) CRX, RAX and THRB;
(ff) MEF2C, OTX2 and RORA;
(gg) NEUROG2, PAX6 and RAX;
(hh) ASCL1, CRX and PAX6;
(ii) FOXP1, NEUROG2, PAX6 and THRB;
(jj) CRX, NEUROD1 and RAX;
(kk) CRX, NEUROG2 and PAX6;
(ll) CRX, NEUROD1, OTX2 and RAX;
(mm) NEUROG2, OTX2 and PAX6;
(nn) CRX and RAX;
(oo) PAX6 and RAX;
(pp) CRX, NEUROG2, OTX2 and RAX; or
(bb) NEUROG2 and PAX6.

In any aspect of a method of the invention described herein, the source cell is a glial cell (e.g. Müller glial cell), and the transcription factors, or biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2;
(k) OTX2, RAX, and PAX6;
(l) ASCL1, NEUROD1, and OTX2;
(m) MEF2C, RAX and THRB;
(n) MEF2C, PAX6 and OTX2;
(o) MEF2C, OTX2 and THRB;
(p) MEF2C, OTX2 and RAX;
(q) ASCL1, CRX and FOXP1;
(r) CRX, NEUROG2, THRB and RAX;
(s) ASCL1, CRX, MEF2C, NEUROD1, OTX2 and THRB;
(t) MEF2C, PAX6 and THRB;
(u) MEF2C, NEUROD1 and PAX6;
(v) ASCL1, OTX2 and RAX;
(w) ASCL1, NEUROG2 and PAX6;
(x) ASCL1, CRX and RAX;
(y) CRX, NEUROD1 and OTX2;
(z) CRX, NEUROG2 and OTX2;
(aa) ASCL1, CRX and NEUROG2;
(bb) CRX, RORA and THRB;
(cc) NEUROD1, OTX2 and RAX;
(dd) CRX, RAX and THRB;
(ee) MEF2C, OTX2 and RORA;
(ff) NEUROG2, PAX6 and RAX;
(gg) ASCL1, CRX and PAX6;
(hh) FOXP1, NEUROG2, PAX6 and THRB;
(ii) CRX, NEUROD1 and RAX;
(jj) CRX, NEUROG2 and PAX6;
(kk) CRX, NEUROD1, OTX2 and RAX;
(ll) NEUROG2, OTX2 and PAX6;
(mm) CRX and RAX;
(nn) PAX6 and RAX;
(oo) CRX, NEUROG2, OTX2 and RAX; or
(pp) NEUROG2 and PAX6.

In any aspect of a method of the invention described herein, the source cell is a glial cell (e.g. Müller glial cell), and the transcription factors, or biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2;
(k) CRX, NEUROD1 and THRB;
(l) OTX2, RAX, and PAX6;
(m) ASCL1, NEUROD1, and OTX2;
(n) MEF2C, RAX and THRB;
(o) MEF2C, PAX6 and OTX2;
(p) MEF2C, OTX2 and THRB;
(q) MEF2C, OTX2 and RAX;
(r) ASCL1, CRX and FOXP1;
(s) CRX, NEUROG2, THRB and RAX;
(t) ASCL1, CRX, MEF2C, NEUROD1, OTX2 and THRB;
(u) MEF2C, PAX6 and THRB;
(v) MEF2C, NEUROD1 and PAX6;
(w) ASCL1, OTX2 and RAX;
(x) ASCL1, NEUROG2 and PAX6;
(y) ASCL1, CRX and RAX;
(z) CRX, NEUROD1 and OTX2;
(aa) CRX, NEUROG2 and OTX2;
(bb) ASCL1, CRX and NEUROG2;
(cc) CRX, RORA and THRB;
(dd) NEUROD1, OTX2 and RAX; or
(ee) CRX, RAX and THRB.

In any aspect of a method of the invention described herein, the source cell is a glial cell (e.g. Müller glial cell), and the transcription factors, or biologically active fragments or variants thereof, are:
(a) ASCL1, NEUROG2 and OTX2;
(b) CRX, NEUROG2, THRB and RAX;
(c) NEUROD1, NEUROG2 and PAX6;
(d) NEUROG2 and PAX6;
(e) ASCL1, OTX2 and PAX6;
(f) CRX, NEUROD1 and THRB;
(g) ASCL1, CRX and RORA;
(h) ASCL1, CRX and ONECUT1;
(i) CRX, OTX2 and RAX;
(j) ASCL1, CRX and NEUROD1;
(k) ASCL1, NEUROG2 and PAX6;
(l) ASCL1, CRX and NEUROG2;
(m) ASCL1, CRX and THRB;
(n) ASCL1, CRX and MEF2C;
(o) CRX, NEUROG2, OTX2 and RAX;
(p) ASCL1, CRX, MEF2C, OTX2, and THRB;
(q) CRX, NEUROG1 and OTX2;
(r) OTX2; RAX and PAX6;
(s) ASCL1; NEUROD1 and OTX2;
(t) CRX; NEUROG2 and OTX2;
(u) ASCL1; CRX and OTX2;
(V) ASCL1, CRX and RAX;
(w) CRX, RORA and THRB;
(x) CRX, RAX and THRB; or
(y) NEUROD1; OTX2 and RAX.

In any aspect of a method of the invention described herein, the source cell is a glial cell (e.g. Müller glial cell), and the transcription factors, or biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2;
(k) CRX, NEUROD1 and THRB;
(l) OTX2, RAX, and PAX6;
(m) ASCL1, NEUROD1, and OTX2;
(n) MEF2C, RAX and THRB;
(o) MEF2C, PAX6 and OTX2;
(p) MEF2C, OTX2 and THRB;
(q) MEF2C, OTX2 and RAX;
(r) ASCL1, CRX and FOXP1;
(s) CRX, NEUROG2, THRB and RAX;
(t) ASCL1, CRX, MEF2C, NEUROD1, OTX2 and THRB;
(u) MEF2C, PAX6 and THRB;
(v) MEF2C, NEUROD1 and PAX6;
(w) ASCL1, OTX2 and RAX; or
(x) ASCL1, NEUROG2 and PAX6.

In any aspect of a method of the invention described herein, the source cell is a glial cell (e.g. Müller glial cell), and the transcription factors, or biologically active fragments or variants thereof, are:
(a) ASCL1, NEUROG2 and OTX2;
(b) CRX, NEUROG2, THRB and RAX;
(c) NEUROD1, NEUROG2 and PAX6;
(d) NEUROG2 and PAX6;
(e) ASCL1, OTX2 and PAX6;
(f) CRX, NEUROD1 and THRB;
(g) ASCL1, CRX and RORA;
(h) ASCL1, CRX and ONECUT1;
(i) CRX, OTX2 and RAX;
(j) ASCL1, CRX and NEUROD1;
(k) ASCL1, NEUROG2 and PAX6;
(l) ASCL1, CRX and NEUROG2; or
(m) ASCL1, CRX and THRB.

In any aspect of a method of the invention described herein, the source cell is a glial cell (e.g. Müller glial cell), and the transcription factors, or biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2;
(k) CRX, NEUROD1 and THRB;
(l) OTX2, RAX, and PAX6;
(m) ASCL1, NEUROD1, and OTX2;
(n) MEF2C, RAX and THRB;
(o) MEF2C, PAX6 and OTX2;
(p) MEF2C, OTX2 and THRB;
(q) MEF2C, OTX2 and RAX; or
(r) ASCL1, CRX and FOXP1.

In any aspect of a method of the invention described herein, the source cell is a glial cell (e.g. Müller glial cell), and the transcription factors, or biologically active fragments or variants thereof, are:
(a) ASCL1, NEUROG2 and OTX2;
(b) CRX, NEUROG2, THRB and RAX;
(c) NEUROD1, NEUROG2 and PAX6;
(d) NEUROG2 and PAX6;
(e) ASCL1, OTX2 and PAX6; or
(f) CRX, NEUROD1 and THRB.

In any aspect of a method of the invention described herein, the source cell is a Müller glial cell, and the transcription factors, or biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2; or
(k) CRX, NEUROD1 and THRB.

In any aspect of a method of the invention described herein, the source cell is a glial cell (e.g. Müller glial cell), and the transcription factors, or biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6; or
(i) ASCL1, CRX and OTX2.

In any aspect of a method of the invention described herein, the source cell is a glial cell (e.g. Müller glial cell), and the transcription factors, or biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C; or
(f) ASCL1, OTX2 and PAX6.

In any aspect of a method of the invention described herein, the source cell is a glial cell (e.g. Müller glial cell), and the transcription factors, or biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1; or
(e) ASCL1, CRX, and MEF2C.

In any aspect of a method of the invention described herein, the source cell is a glial cell (e.g. Müller glial cell), and the transcription factors, or biologically active fragments or variants thereof, are:
(a) ASCL1, CRX and ONECUT1;
(b) ASCL1, OTX2 and PAX6; or
(c) ASCL1, CRX and RORA.

In any aspect of a method of the invention described herein, the source cell is a glial cell (e.g. Müller glial cell), and the transcription factors, or biologically active fragments or variants thereof, are:
(a) ASCL1, CRX and ONECUT1;
(b) ASCL1, CRX and THRB; or
(c) ASCL1, CRX and RORA.

In any aspect of a method of the invention described herein, the source cell is a glial cell (e.g. Müller glial cell), and the transcription factors, or biologically active fragments or variants thereof, are:
(a) ASCL1, NEUROG2 and OTX2; or
(b) CRX, NEUROG2, THRB and RAX.

In any aspect of a method of the invention described herein, the source cell is a glial cell (e.g. Müller glial cell), and the transcription factors, or biologically active fragments or variants thereof, are:
(a) ASCL1, CRX and ONECUT1; or
(b) ASCL1, CRX and THRB.

Preferably, the at least one characteristic of the cone photoreceptor cell is up-regulation of any one or more target cell markers and/or change in cell morphology. Relevant markers are described herein and known to those in the art. Exemplary markers for the cone photoreceptor cells include:
ARR3, CNGB3, GNAT2, GNGT2, GRK7, GUCA1C, PDE6C, PDE6H, RXRG, THRB, OPN1LW, OPN1MW and OPN1SW;
an electrophysiological response in a photopic condition, for example, as described in the Examples.

In any embodiment, the cone photoreceptor cell, or cone photoreceptor-like cell, produced or generated from a method or use described herein exhibits a detectable level of any one, 2, 3, 4, 5, 6, 7, 8, 9, 10, 11 or 12 of the following markers ARR3, CNGB3, GNAT2, GNGT2, GRK7, GUCA1C, PDE6C, PDE6H, RXRG, THRB, OPN1LW, OPN1MW and OPN1SW. The cone photoreceptor cell, or cone photoreceptor-like cell may have a detectable level of OPN1LW, OPN1MW or OPN1SW.

Additional examples of photoreceptor markers include the opsins that are light-detecting molecules. For example, rhodopsin (rod photoreceptor cells), red/green opsin (cone photoreceptor cells), blue opsin (cone photoreceptor cells), and recoverins (rod photoreceptor cells, cone photoreceptor cells).

In any aspect, the combination of transcription factors one or more of those selected from NEUROG2, CRX, RAX, RORA, NEUROD1, OTX2, ASCL1, PAX6, THRB, MEF2C, FOXP1 and ONECUT1, wherein the combination results in a cone photoreceptor, or cone photoreceptor-like, cells with a fold increase in opsin mRNA expression of equal to, or greater than, 2, 3, 4, 5, 6, 7, 8, 9, 10, 11, 12, 13, 14, 15, 16, 17 or 18 fold compared to the opsin expression in the source cell type. Preferably, the fold increase is equal to, or greater than, 4, 5, 6, 7, 8, 9, 10, 11, 12, 13, 14, 15, 16, 17 or 18 fold compared to the opsin expression in the source cell type. Preferably, the fold increase is equal to, or greater than, 5, 6, 7, 8, 9, 10, 11, 12, 13, 14, 15, 16, 17 or 18 fold compared to the opsin expression in the source cell type. Preferably, the fold increase is equal to, or greater than, 6, 7, 8, 9, 10, 11, 12, 13, 14, 15, 16, 17 or 18 fold compared to the opsin expression in the source cell type. Preferably, the fold increase is equal to, or greater than, 7, 8, 9, 10, 11, 12, 13, 14, 15, 16, 17 or 18 fold compared to the opsin expression in the source cell type. Preferably, the fold increase is equal to, or greater than, 4, 5, 6, 9, 10, 11, 12, 13, 14, 15, 16, 17 or 18 fold compared to the opsin expression in the source cell type. Preferably, the fold increase is equal to, or greater than, 10, 11, 12, 13, 14, 15, 16, 17 or 18 fold compared to the opsin expression in the source cell type. Most preferably, the fold increase is equal to, or greater than, Preferably, the fold increase is equal to, or greater than, 13, 14, 15, 16, 17 or 18 fold compared to the opsin expression in the source cell type. The opsin may be OPN1LW/MW and/or OPN1SW.

As used herein, an opsin may be encoded by the gene OPN1LW/MW or the gene OPN1SW.

In any aspect of the present invention, the source cell is a human cell. Where the source cell is a Müller glial cell, it may be a human Müller glial cell.

Typically, conditions suitable for photoreceptor cell differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 2.

In any aspect of the present invention, the cells may be contacted with Trichostatin A during the transfecting or culturing step.

In another aspect, the present invention also provides a cell exhibiting at least one characteristic of a cone photoreceptor cell produced by a method as described herein.

In any method described herein, the method may further include the step of expanding the cells exhibiting at least one characteristic of a cone photoreceptor cell to increase the proportion of cells in the population exhibiting at least one characteristic of a cone photoreceptor cell. The step of expanding the cells may be in culture for a sufficient time and under conditions for generating a population of cells as described below.

In any method described herein, the method may further include the step of administering the cells, or cell population including a cell, exhibiting at least one characteristic of a cone photoreceptor cell, to an individual.

The present invention also provides a population of cells, wherein at least 0.01%, at least 0.02%, at least 0.03%, at least 0.04%, at least 0.05, at least 0.06%, at least 0.07%, at least 0.08%, at least 0.09%, at least 0.1%, at least 0.15%, at least 0.2%, at least 0.25%, at least 0.3%, at least 0.35%, at least 0.4%, at least 0.45%, at least 0.5, at least 0.6%, at least 0.7%, at least 0.8%, at least 0.9%, at least 1%, at least 2%, at least 3%, at least 4% or at least 5% of cells exhibit at least one characteristic of a cone photoreceptor cell and those cells are produced by a method as described herein. Preferably, at least 5% at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or at least 100% of the cells in the population exhibit at least one characteristic of a cone photoreceptor cell. The present invention also provides a population of cells, wherein 0.01%, 0.02%, 0.03%, 0.04%, 0.05, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9%, 1%, 2%, 3%, 4% or 5% of cells exhibit at least one characteristic of a cone photoreceptor cell and those cells are produced by a method as described herein. Preferably, 5% 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of a cone photoreceptor cell.

The present invention also relates to kits for producing a cell exhibiting at least one characteristic of a cone photoreceptor cell as disclose herein. In some embodiments, a kit comprises one or more nucleic acids having one or more nucleic acid sequences encoding a transcription factor described herein or biological active fragment variant thereof, including the specific combinations referred to in (a) to (ee) herein. Preferably, the kit can be used with a source cell referred to herein. In some embodiments, the kit further comprises instructions for reprogramming a source cell to a cell exhibiting at least one characteristic of a cone photoreceptor cell according to the methods as disclosed herein. Preferably, the present invention provides a kit when used in a method of the invention described herein.

In another aspect, the present invention relates to a composition comprising at least one source cell as described herein and at least one agent which increases the expression of genes encoding one or more transcription factors in the source cell. Further, the transcription factor may be any one or more described herein, including the combinations reference to in (a) to (ee) herein.

Typically, the gene expression, or amount, of a transcription factor as described herein is increased by contacting the cell with an agent which increases the expression of the transcription factor. Preferably, the agent is selected from the group consisting of: a nucleotide sequence, a protein, an aptamer and small molecule, ribosome, RNAi agent and peptide-nucleic acid (PNA) and analogues or biologically active fragments or variants thereof. Preferably, the agent is exogenous. In a preferred embodiment, the agent or agents are transgene(s) or CRISPR components, such as those described herein, that induce endogenous gene activation. For example, a CRISPR activation system, and components thereof including sgRNAs, such as that described herein, is contemplated as an agent that increases the expression of one or more transcription factors.

Typically, the gene expression, or amount, of a transcription factor as described herein is increased by introducing at least one nucleic acid comprising a nucleotide sequence encoding a transcription factor, or encoding a functional fragment thereof, in the cell. Preferably, the nucleotide sequence encoding a transcription factor is at least 70%, 80%, 85%, 90%, 91%, 92%, 93%, 94%, 95%, 96%, 97%, 98%, or 99% identical to a sequence with an accession number listed in Table 1.

The gene expression, or amount, of a transcription factor as described herein may also be increased by introducing at least one nucleic acid (such as an sgRNA) for use in a CRISPR activation system, for increasing the expression of the gene encoding the transcription factor.

Preferably, the nucleic acid further includes a heterologous promoter. Preferably, the nucleic acid is in a vector, such as a viral vector or a non-viral vector. In one embodiment, the nucleic acid may be RNA, preferably mRNA, most preferably synthetic mRNA. Preferably, the vector is a viral vector comprising a genome that does not integrate into the host cell genome. The viral vector may be a retroviral vector, AAV vector, baculoviral vector or a lentiviral vector.

In another aspect, the present invention relates to a nucleic acid or vector comprising a nucleic acid as described herein that may include one or more nucleotide sequences encoding one or more transcription factors as described herein. Preferably, the nucleic acid or vector encodes one or more sets of transcription factors as described herein, including in (a) through to (ee) above, and Table 3 below. In one embodiment, the nucleic acid or vector comprises one or more of the sequences referred to above in Table 1 or a sequence encoding any one or more of the amino acid sequences listed in Table 1. In another embodiment, the nucleic acid or vector is any one as described herein.

In another aspect, the present invention relates to a CRISPR activation system for increasing the expression of the gene encoding one or more of transcription factors described herein. Preferably the CRISPR activation system results in increasing the expression of one or more sets of transcription factors as described herein, including in (a) through to (qq) above, and Table 3 below. In one embodiment, the CRISPR activation system comprises the sgRNAs described herein, including, sgRNAs that target genes selected from the group consisting of: CRX, MEF2C, THRB, RAX, NEUROD1, RORA, OTX2, NEUROG2/NGN2, PAX6, FOXP1, ASCL1 and ONECUT1 (On).

In another aspect, the present invention relates to an in vitro or ex vivo cell comprising a nucleic acid or vector of the invention as described herein.

In any aspect of the present invention, the method as described herein may have one or more, or all, steps performed in vitro, ex vivo or in vivo.

In another aspect, the present invention provides a method of treating a condition associated with or caused by degeneration, or loss, of cone photoreceptor cells in an individual in need thereof, the method comprising administering to the individual a cell or cell population generated in vitro or ex vivo by any method described herein.

In another aspect, the present invention provides a use of a cell or cell population generated in vitro or ex vivo by any method described herein in the manufacture of a medicament for the treatment of a condition associated with or caused by degeneration, or loss, of cone photoreceptor cells in an individual in need thereof.

In another aspect, the present invention provides a cell or cell population generated in vitro or ex vivo by any method described herein for use in the treatment of a condition associated with or caused by degeneration, or loss, of cone photoreceptor cells in an individual in need thereof.

In any embodiment, the nucleic acid or vector comprises or consists of an expression construct. In some embodiment, the expression construct comprises one of more features of an AAV, rAAV, lentiviral or baculovirus vector or synthetic mRNA. Preferably, the expression construct comprises one or more features of an AAV, or rAAV, vector of the invention as described herein.

In another aspect, the present invention also provides a recombinant vector comprising an expression construct as described herein. The recombinant vector may be a recombinant AAV (rAAV) vector.

The promoter in the expression construct, or any other aspect of the invention described herein, may be any nucleotide sequence that is capable of inducing RNA polymerase to bind to and transcribe the coding sequence. The promoter may be a ubiquitous promoter or a glial cell-specific promoter

In one preferred embodiment, the promoter is the CAG promoter. The CAG promoter preferably comprises the cytomegalovirus (CMV) early enhancer element, the promoter, the first exon and the first intron of chicken beta-actin (CBA) gene and the splice acceptor of the rabbit beta-globin gene.

In one embodiment the nucleotide sequence encoding the CMV early enhancer element is 245 bp long, and is referred to herein as SEQ ID NO: 1. Preferably, the CMV early enhancer element comprises a nucleotide sequence substantially as set out in SEQ ID NO: 1, or a fragment or variant thereof.

In one embodiment the nucleotide sequence encoding the GFAP promoter is 681 bp long, and is referred to herein as SEQ ID NO: 2. Preferably, the promoter comprises a nucleotide sequence substantially as set out in SEQ ID NO: 2, or a fragment or variant thereof.

In one embodiment the nucleotide sequence encoding the first intron of chicken-beta actin gene (CBA) is 408 bp long, and is referred to herein as SEQ ID NO: 3. Preferably, the first intron of CBA comprises a nucleotide sequence substantially as set out in SEQ ID NO: 3, or a fragment or variant thereof.

Preferably, the expression construct, or any other aspect of the invention described herein, comprises a nucleotide sequence encoding a Kozak sequence, which enhances transcription factor expression or a biologically active fragment or variant thereof. Preferably, the Kozak coding sequence is disposed 5′ of the nucleotide sequence encoding the one or more sets of transcription factors as described herein, including in (a) through to (qq) above, and Table 3 below, or biologically active fragments or variants thereof.

In one embodiment the nucleotide sequence encoding the Kozak sequence is 10 bp long, and is referred to herein as SEQ ID NO: 4. Preferably, the Kozak sequence comprises a nucleotide sequence substantially as set out in SEQ ID NO: 4, or a fragment or variant thereof.

Preferably, the expression construct, or any other aspect of the invention described herein, comprises a nucleotide sequence encoding a Woodchuck Hepatitis Virus Post-transcriptional Regulatory Element (WPRE), which enhances expression of one or more sets of transcription factors as described herein, including in (a) through to (qq) above, and Table 3 below or biologically active fragments or variants thereof. Preferably, the WPRE coding sequence is disposed 3′ of the nucleotide sequence encoding the one or more sets of transcription factors as described herein, including in (a) through to (qq) above, and Table 3 below or biologically active fragments or variants thereof.

In one embodiment the nucleotide sequence encoding WPRE is 593 bp long, and is referred to herein as SEQ ID NO: 5. Preferably, the WPRE comprises a nucleotide sequence substantially as set out in SEQ ID NO: 5, or a fragment or variant thereof.

Preferably, the expression construct, or any other aspect of the invention described herein, comprises a nucleotide sequence encoding a bovine growth hormone (bGH) polyA tail. Preferably, the bGH polyA tail coding sequence is disposed 3′ of the nucleotide sequence encoding the one or more sets of transcription factors as described herein, including in (a) through to (qq) above, and Table 3 below or biologically active fragments or variants thereof, and preferably 3′ of the WPRE coding sequence.

In one embodiment, the nucleotide sequence encoding the bovine growth hormone (bGH) polyA tail is 269 bp long, and is referred to herein as SEQ ID NO: 6. Preferably, the bovine growth hormone polyA tail comprises a nucleotide sequence substantially as set out in SEQ ID NO: 6, or a fragment or variant thereof.

In any embodiment, the expression construct comprises AAV Inverted Terminal Repeats (ITRs), for example AAV ITRs flanking the nucleotide sequence encoding the one or more sets of transcription factors as described herein, including in (a) through to (qq) above, and Table 3 below or biologically active fragments or variants thereof.

Preferably, the expression construct, or any other aspect of the invention described herein, comprises left and/or right ITRs. Preferably, each ITR is disposed at the 5′ and/or 3′ end of the construct.

In one embodiment, the nucleotide sequence of the left ITR is represented herein as SEQ ID NO: 7.

In one embodiment, the nucleotide sequence of the right ITR is represented herein as SEQ ID NO: 8.

In another aspect, the present invention also provides an adeno-associated viral (AAV) vector, lentiviral vector, baculoviral vector or mRNA (e.g. synthetic mRNA) comprising a nucleotide sequence encoding the one or more sets of transcription factors as described herein, including in (a) through to (qq) above, and Table 3 below or biologically active fragments or variants thereof. Typically, the nucleotide sequence encoding the one or more sets of transcription factors as described herein, including in (a) through to (qq) above, and Table 3 below or biologically active fragments or variants thereof is flanked by two AAV Inverted Terminal Repeats (ITRs).

In any embodiment, the AAV vector, lentiviral vector or baculoviral vector is recombinant, synthetic, purified, or substantially purified.

In some embodiments the AAV vector is a recombinant AAV (rAAV) vector. The rAAV may be a naturally occurring vector or a vector with a hybrid AAV serotype. The rAAV may be AAV-1, AAV-2, AAV-3, AAV-4, AAV-5, AAV-6, AAV-7, AAV-8, AAV-9, AAV-10, AAV-11, AAV-12, AAV-13, ShH10 and ShH10Y.

The recombinant AAV vector may be a bioengineered vector. The rAAV may be Anc80, DJ, DJ/8, KP1, KP2, KP3, LK01, LK02, LK03, LK19, NP6, NP22, NP40, NP59, NP66, NP84, NP94, rh10, 2i8, 7m8, PHP.eB and AAV2 Retro.

The recombinant vector may be SYD01, SYD03, SYD09, HRS1, HRS19, HRS5, CD15, T33, CMRI-01, CMRI-02, CMRI-03, CMRI-04, CMRI-05, CMRI-06, CMRI-07 and CMRI-08.

Preferably, however, the rAAV is ShH10 or ShH10Y.

Advantageously, ShH10 and ShH10Y, derived from an AAV6 parent serotype, is capable of efficient, selective Müller cell infection through intravitreal injection. ShH10 and ShH10Y also shows significantly improved transduction relative to AAV2 (>60%) and AAV6.

The term “recombinant (rAAV) vector” as used herein means a recombinant AAV-derived nucleic acid containing at least one terminal repeat sequence.

Preferably, the expression construct, recombinant plasmid vector or any other aspect of the invention described herein comprises at least one stuffer sequence, preferably one or more than one of the following stuffer sequences described below. Preferably, the recombinant plasmid vector comprises a first, second, third, fourth, fifth, sixth and/or seventh stuffer sequence, preferably wherein the first, second, third, fourth, fifth, sixth and seventh stuffer sequences are as described herein (e.g. SEQ ID NO: 9-15.

In one embodiment, the first stuffer sequence is represented herein as SEQ ID No: 9.

In one embodiment, the second stuffer sequence is represented herein as SEQ ID No: 10.

In one embodiment, the third stuffer sequence is represented herein as SEQ ID No: 11.

In one embodiment, the fourth stuffer sequence is represented herein as SEQ ID No: 12.

In one embodiment, the fifth stuffer sequence is represented herein as SEQ ID No: 13.

In one embodiment, the sixth stuffer sequence is represented herein as SEQ ID No: 14.

In one embodiment, the sixth stuffer sequence is represented herein as SEQ ID No: 15.

Preferably, the expression construct, recombinant plasmid vector or any other aspect of the invention described herein comprises an antibiotic resistance gene. Typically, the antibiotic resistance gene is a nucleotide sequence encoding a kanamycin resistance gene.

In one embodiment, the nucleotide sequence encoding the kanamycin resistance gene is 816 bp long, and is referred to herein as SEQ ID NO: 16.

Preferably, the expression construct, recombinant plasmid vector or any other aspect of the invention described herein comprises a pUC origin. Typically, nucleotide sequence encoding the pUC origin is 668 bp long, and is referred to herein as SEQ ID No: 17.

In any embodiment, the AAV vector, lentiviral vector, baculoviral vector or synthetic mRNA further comprises one or more regulatory sequences (e.g. promoter) that allows, or causes, expression of the one or more sets of transcription factors as described herein, including in (a) through to (ee) above, and Table 3 below or biologically active fragments or variants thereof in glial cells, preferably retinal glial cells.

Preferably, the promoter is a ubiquitous promoter or a glial cell-specific promoter. In any embodiment, the nucleotide sequence encoding the one or more sets of transcription factors as described herein, including in (a) through to (qq) above, and Table 3 below or biologically active fragments or variants thereof is operably linked to the promoter.

Examples of ubiquitous promoters include a CAG promoter. The CAG promoter preferably comprises the cytomegalovirus (CMV) early enhancer element, the promoter, the first exon and the first intron of chicken beta-actin (CBA) gene and the splice acceptor of the rabbit beta-globin gene.

Examples of glial cell-specific promoters include the promoters for GFAP, GLAST and RLBP1 genes and/or combinations of glial cell-specific transcription factor regulatory elements.

In some embodiments, the AAV vector comprises a CMV promoter, for example as described herein. In some embodiments, the AAV vector comprises a Kozak sequence, for example as described herein. In some embodiments, the vector comprises one or more ITR sequence flanking the vector portion encoding the one or more sets of transcription factors as described herein, including in (a) through to (qq) above, and Table 3 below or biologically active fragments or variants thereof, for example as described herein. In some embodiments, the vector comprises a polyadenylation sequence. In some embodiments, the vector comprises a selective marker. Preferably, the selective marker is an antibiotic-resistance gene, such as an ampicillin-resistance gene or a kanamycin-resistance gene.

In any embodiment, the ITR, or each ITR if two or more, is a wildtype AAV ITR sequence, or ITR as described herein.

In one embodiment, the present invention provides a recombinant adeno-associated virus (AAV) vector comprising a nucleic acid comprising, in 5′ to 3′ order:
(a) a 5′ AAV ITR, for example SEQ ID NO: 7 or 8;
(b) a CMV enhancer, for example SEQ ID NO: 1;
(c) a glial cell-specific promoter;
(d) a transgene encoding the one or more sets of transcription factors as described herein, including in (a) through to (qq) above, and Table 3 below or biologically active fragments or variants thereof;
(e) a WPRE, for example SEQ ID NO: 5;
(f) a Bovine Growth Hormone polyA signal tail, for example SEQ ID NO: 6; and
(g) a 3′AAV ITR, for example SEQ ID NO: 7 or 8.

In another aspect, the present invention provides a recombinant adeno-associated virus (rAAV) comprising:
(i) an AAV capsid protein; and
(ii) an AAV vector of the invention as described herein.

In one embodiment, the AAV capsid protein is a ShH10 or ShH10Y capsid protein.

In any embodiment, the rAAV may be a AAV variant or mutant as described herein.

In another aspect, the present invention provides a pharmaceutical composition comprising an isolated nucleic acid of the invention as described herein, a genetic construct of the invention as described herein an AAV vector of the invention as described herein, or a recombinant AAV of the invention as described herein, and a pharmaceutically acceptable carrier, diluent or excipient.

In another aspect, the present invention provides a plasmid comprising isolated nucleic acid comprising an expression construct of the invention as described herein, or an AAV vector of the invention as described herein.

In another aspect, the present invention provides a Baculovirus vector comprising a nucleic acid of the invention as described herein.

In another aspect, the present invention provides a cell comprising:
(i) a first vector encoding one of more adeno-associated virus rep protein and/or one or more adeno-associated virus cap protein; and
(ii) a second vector comprising a nucleotide sequence encoding the one or more sets of transcription factors as described herein, including in (a) through to (qq) above, and Table 3 below or biologically active fragments or variants thereof.

In one embodiment, the first vector is a plasmid and the second vector is a plasmid. In another embodiment, the first vector is a Baculovirus vector and the second vector is a Baculovirus vector.

Typically, the cell is a mammalian cell, preferably the mammalian cell is a HEK293 cell. Alternatively, the cell is an insect cell, preferably the insect cell is a SF9 cell.

In another aspect, the present inventions provides a method of producing an AAV of the invention as described herein, the method comprising:
(i) delivering to a cell a first vector encoding one or more adeno-associated virus rep protein and/or one or more adeno-associated cap protein, and a recombinant AAV vector comprising a expression cassette comprising a nucleotide sequence that encodes the one or more sets of transcription factors as described herein, including in (a) through to (qq) above, and Table 3 below or biologically active fragments or variants thereof;
(ii) culturing the cells under conditions allowing for packaging the AAV; and
(iii) harvesting the cultured host cell or culture medium for collection of the AAV.

In another aspect, the present invention provides a method of decreasing progression of or ameliorating vision loss associated with cone dystrophy in a subject, the method comprising administering to the subject an isolated nucleic acid of the invention as described herein, a genetic construct of the invention as described herein an AAV vector of the invention as described herein, a recombinant AAV of the invention as described herein, or a pharmaceutical composition of the invention as described herein, thereby of decreasing progression of or ameliorating vision loss associated with or cause by degeneration, or loss, of cone photoreceptor cells.

In another aspect, the present invention provides use of an isolated nucleic acid of the invention as described herein, an AAV vector of the invention as described herein, a recombinant AAV of the invention as described herein, or a pharmaceutical composition of the invention as described herein, in the manufacture of a medicament for decreasing progression of or ameliorating vision loss associated with or cause by degeneration, or loss, of cone photoreceptor cells in a subject.

In another aspect, the present invention provides an isolated nucleic acid of the invention as described herein, an AAV vector of the invention as described herein, a recombinant AAV of the invention as described herein, or a pharmaceutical composition of the invention as described herein, for use in decreasing progression of or ameliorating vision associated with or cause by degeneration, or loss, of cone photoreceptor cells in a subject.

In any aspect, preferably the subject is a human.

In any aspect or embodiment, the condition associated with or cause by degeneration, or loss, of cone photoreceptor cells may also be referred to as a cone cell disorder. The degeneration, or loss, of cone photoreceptor cells is associated with or causes changes in vision, typically a reduction in vision.

In some embodiments, the cone cell disorder is a retinal degenerative disorder. In certain embodiments, the retinal degenerative disorder is selected from the group consisting of achromatopsia, blue cone monochromacy, a protan defect, a deutan defect, and a tritan defect. In some embodiments, the cone cell disorder is a macular dystrophy or retinal dystrophy. The macular dystrophy may be selected from the group consisting of Stargardt's macular dystrophy, cone dystrophy (including rod-cone dystrophy and cone-rod dystrophy), Spinocerebellar ataxia type 7, and Bardet-Biedl syndrome-1. Preferably, the macular dystrophy is Stargardt's macular dystrophy or cone-rod dystrophy. In some embodiments, the cone cell disorder is a vision disorder of the central macula or a retinal dystrophy. In certain embodiments, vision disorder of the central macula or retinal dystrophy is selected from the group consisting of age-related macular degeneration, macular telangiectasia, retinitis pigmentosa, diabetic retinopathy, retinal vein occlusions, glaucoma, choroideremia, Sorsby's fundus dystrophy, adult vitelliform macular dystrophy, Best's disease, Leber's congenital amaurosis, and X-linked retinoschisis. Preferably, the vision disorder is retinitis pigmentosa, age-related macular degeneration or diabetic retinopathy.

In any embodiment, the subject has been diagnosed with a condition associated with or cause by degeneration, or loss, of cone photoreceptor cells as described herein. Preferably, the individual has been diagnosed with a cone dystrophy. The individual may have been diagnosed with progressive cone dystrophy or stationary cone dystrophy. The cone dystrophy may be a rod-cone dystrophy or a cone-rod dystrophy.

In some such embodiments, the method further comprises detecting a change in the condition or disorder symptoms. Including any symptom described herein. In some such embodiments, the change comprises a stabilization in the health of the existing or reprogrammed cone cells and/or a reduction in the rate of visual acuity loss of the subject. In certain such embodiments, the change comprises an improvement in in the visual acuity of the subject.

In some such embodiments, the method further comprises detecting a change in the condition or disorder symptoms, wherein the change comprises an increase in the ability of the subject to perceive a colour.

In any aspect of the present invention, the isolated nucleic acid of the invention as described herein, an AAV vector of the invention as described herein, a recombinant AAV of the invention as described herein, or a pharmaceutical composition of the invention as described herein, is administered to the subject via the retina. In other words, the isolated nucleic acid of the invention as described herein, an AAV vector of the invention as described herein, a recombinant AAV of the invention as described herein, or a pharmaceutical composition of the invention as described herein is administered by retinal administration. Typically, the retinal administration is by retinal injection (e.g. intravitreal or subretinal injection) into an affected eye of said subject.

In another aspect, the present invention provides for a composition comprising any of the AAV vectors or rAAV of the invention as disclosed herein and a pharmaceutically acceptable carrier, excipient or diluent.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Experimental setup for genome-wide CRISPRa screening for genes that promote reprogramming of human Muller glial (MG) cells to cones.

FIG. 2. Genome-wide CRISPRa screens. A) Genome-wide CRISPRa screens identified genes for iCone reprogramming, including top hit NEUROG2. B) Gene ontology analysis showed candidate genes are related to photoreceptor signalling and functions.

FIG. 3. The identified transcription factors form a key transcriptional network with NEUROG2 as a core factor (arrow).

FIG. 4. Schematic of in vitro reprogramming of human MG cells (MIOM1) into iCones.

FIG. 5. Characterisation of photoreceptors by iCones reprogramming. A) iCones expressed L/M-opsins (OPN1LW/MW-DsRed+). B) Gene expression profiling showed iCones upregulated cone marker genes. C) Multi-electrode array showed iCones possess functional electrophysiology.

FIG. 6. Initial screening of transcription factor cocktails for iCone reprogramming. Dotted line mark 2-fold increase compared to control. Ng: NEUROG2; C: CRX; R: RAX; Roa: RORA; N: NEUROD1; O: OTX2; A: ASCL1; P: PAX6; T: THRB, M: MEF2C; F: FOXP1; On: ONECUT1.

FIG. 7. Optimisation of transcription factor cocktails for reprogramming human MG cells into iCones first in A) primary screening and subsequently validated in B) secondary screening (n=3-4 biological repeats). C: CRX; M: MEF2C; T: THRB; R: RAX: N: NEUROD1; Roa: RORA; O: OTX2; P: PAX6; F: FOXP1; A: ASCL1; Ng: NEUROG2; On: ONECUT1.

FIG. 8. A) Schematic of in vivo reprogramming testing in a rat retinitis pigmentosa (RP) model with photoreceptor degeneration (P23H3). Intravitreal injection of adeno-associated viruses (AAV) carrying iCone genes were performed in P23H3 rats, and visual response were analysed using electroretinogram (ERG) 4 weeks after treatment. B) Schematic of AAV vector used to deliver individual iCone gene driven by the Muller glia (MG)-specific promoter GFAP and generated using the MG-specific targeting AAV serotype ShH10Y.

FIG. 9. ERG analysis of P23H3 rats following injection of AAV carrying iCone genes Ascl1+Crx+Ng (ACNg), highlighting the functional improvement in visual response following AAV delivery of iCone genes. The a-wave depicting photoreceptor function (A,C) and the b-wave depicting bipolar function (B,D) were normalised before and after treatment for the individual eye. Naïve (untreated) controls and sham controls with PBS injection were used as negative controls. ***: p>0.001.

FIG. 10. Immunohistochemistry analysis showed localised increase in the thickness of the outer nuclear layer (ONL) in P23H3 rats (marked by white arrows) following treatment with AAV delivery of Ascl1+Crx+Neurog2 (ACNg) compared to untreated control. DAPI was used as a nuclear counterstain together with the photoreceptor marker Recoverin. INL: inner nuclear layer; ONL: outer nuclear layer.

### SEQUENCE LISTING

| | SEQ | |
|---|---|---|
| Description | ID NO | Nucleotide or amino acid sequence |
| CMV early enhancer | 1 | CCATTGACGTCAATAATGACGTATGTTCC |
| element | | CATAGTAACGCCAATAGGGACTTTCCATT |
| | | GACGTCAATGGGTGGAGTATTTACGGTAA |
| | | ACTGCCCACTTGGCAGTACATCAAGTGTAT |
| | | CATATGCCAAGTACGCCCCCTATTGACGTC |
| | | AATGACGGTAAATGGCCCGCCTGGCATTAT |
| | | GCCCAGTACATGACCTTATGGGACTTTCCT |
| | | ACTTGGCAGTACATCTACGTATTAGTCATC |
| | | GCTATTAC |
| Promoter | 2 | AACATATCCTGGTGTGGAGTAGGGGACGCTG |
| | | CTCTGACAGAGGCTCGGGGGCCTGAGCTGG |
| | | CTCTGTGAGCTGGGGAGGAGGCAGACAGCC |
| | | AGGCCTTGTCTGCAAGCAGACCTGGCAGCAT |
| | | TGGGCTGGCCGCCCCCCAGGGCCTCCTCTTC |
| | | ATGCCCAGTGAATGACTCACCTTGGCACAGAC |
| | | ACAATGTTCGGGGTGGGCACAGTGCCTGCTTC |
| | | CCGCCGCACCCCAGCCCCCCTCAAATGCCTTC |
| | | CGAGAAGCCCATTGAGCAGGGGGCTTGCATTG |
| | | CACCCCAGCCTGACAGCCTGGCATCTTGGGAT |
| | | AAAAGCAGCACAGCCCCCTAGGGGCTGCCCTT |
| | | GCTGTGTGGCGCCACCGGCGGTGGAGAACAA |
| | | GGCTCTATTCAGCCTGTGCCCAGGAAAGGGGA |
| | | TCAGGGGATGCCCAGGCATGGACAGTGGGTG |
| | | GCAGGGGGGGAGAGGAGGGCTGTCTGCTTCC |
| | | CAGAAGTCCAAGGACACAAATGGGTGAGGGGA |
| | | GAGCTCTCCCCATAGCTGGGCTGCGGCCCAAC |
| | | CCCACCCCCTCAGGCTATGCCAGGGGGTGTTG |
| | | CCAGGGGCACCCGGGCATCGCCAGTCTAGCCC |
| | | ACTCCTTCATAAAGCCCTCGCATCCCAGGAGCG |
| | | AGCAGAGCCAGAGCAGGTTGGAGAGGAGACGC |
| | | ATCACCTCCGCTGCTCGC |
| First intron of | 3 | GGAGTCGCTGCGCGCTGCCTTCGCCCCGTGCCCC |
| chicken-beta actin | | GCTCCGCCGCCGCCTCGCGCCGCCCGCCCCGGCT |
| gene (CBA) | | CTGACTGACCGCGTTACTCCCACAGGTGAGCGGG |
| | | CGGGACGGCCCTTCTCCTCCGGGCTGTAATTAGCG |
| | | CTTGGTTTAATGACGGCTTGTTTCTTTTCTGTGGCTG |
| | | CGTGAAAGCCTTGAGGGGCTCCGGGAGGGCCCTTT |
| | | GTGCGGGGGGAGCGGCTCGGGGCTGTCCGCGGGG |
| | | GGACGGCTGCCTTCGGGGGGGACGGGGCAGGGCG |
| | | GGGTTCGGCTTCTGGCGTGTGACCGGCGGCTCTAG |
| | | AGCCTCTGCTAACCATGTTCATGCCTTCTTCTTTTTC |
| | | CTACAGCTCCTGGGCAACGTGCTGGTTATTGTGCTG |
| | | TCTCATCATTTTGGCAAAGAATT |
| Kozak sequence | 4 | GGCACCATGG |
| WPRE sequence | 5 | ATCGATAATCAACCTCTGGATTACAAAATTTGTGAAAG |
| | | ATTGACTGGTATTCTTAACTATGTTGCTCCTTTTACGCT |
| | | ATGTGGATACGCTGCTTTAATGCCTTTGTATCATGCTAT |
| | | TGCTTCCCGTATGGCTTTCATTTTCTCCTCCTT |
| | | GTATAAATCCTGGTTGCTGTCTCTTTATGAGG |
| | | AGTTGTGGCCCGTTGTCAGGCAACGTGGCGT |
| | | GGTGTGCACTGTGTTTGCTGACGCAACCCCC |
| | | ACTGGTTGGGGCATTGCCACCACCTGTCAGC |
| | | TCCTTTCCGGGACTTTCGCTTTCCCCCTCCCT |
| | | ATTGCCACGGCGGAACTCATCGCCGCCTGCC |
| | | TTGCCCGCTGCTGGACAGGGGCTCGGCTGTT |
| | | GGGCACTGACAATTCCGTGGTGTTGTCGGGG |
| | | AAATCATCGTCCTTTCCTTGGCTGCTCGCCTG |
| | | TGTTGCCACCTGGATTCTGCGCGGGACGTCC |
| | | TTCTGCTACGTCCCTTCGGCCCTCAATCCAGC |
| | | GGACCTTCCTTCCCGCGGCCTGCTGCCGGCT |
| | | CTGCGGCCTCTTCCGCGTCTTCGCCTTCGCC |
| | | CTCAGACGAGTCGGATCTCCCTTTGGGCCGC |
| | | CTCCCC |
| bovine growth | 6 | CGCTGATCAGCCTCGACTGTGCCTTCTAGTTG |
| hormone (bGH) | | CCAGCCATCTGTTGTTTGCCCCTCCCCCGTGC |
| polyA tail sequence | | CTTCCTTGACCCTGGAAGGTGCCACTCCCACT |
| | | GTCCTTTCCTAATAAAATGAGGAAATTGCATCG |
| | | CATTGTCTGAGTAGGTGTCATTCTATTCTGGGG |
| | | GGTGGGGTGGGGCAGGACAGCAAGGGGGAGG |
| | | ATTGGGAAGACAATAGCAGGCATGCTGGGGATG |
| | | CGGTGGGCTCTATGGCTTCTGAGGCGGAAAGAA |
| | | CCAGCTGGGG |
| Left ITR | 7 | CCTGCAGGCAGCTGCGCGCTCGCTCGCTCACTG |
| | | AGGCCGCCCGGGCAAAGCCCGGGCGTCGGGCG |
| | | ACCTTTGGTCGCCCGGCCTCAGTGAGCGAGCGA |
| | | GCGCGCAGAGAGGGAGTGGCCAACTCCATCACTA |
| | | GGGGTTCCT |
| Right ITR | 8 | AGGAACCCCTAGTGATGGAGTTGGCCACTCCCTCT |
| | | CTGCGCGCTCGCTCGCTCACTGAGGCCGGGCGAC |
| | | CAAAGGTCGCCCGACGCCCGGGCTTTGCCCGGGC |
| | | GGCCTCAGTGAGCGAGCGAGCGCGCAGCTGCCTG |
| | | CAGG |
| First stuffer | 9 | GCGGCCAATTCAGTCGATAACTATAACGGT |
| sequence | | CCTAAGGTAGCGATTTAAATCCCGCCATGC |
| | | TACTTATCTACGTAGCCATGCTCTAGGTA |
| Second stuffer | 10 | TCTAGCGGCCGCGAATTCGATATCAAGCTT |
| sequence | | |
| Third stuffer | 11 | GGCGGCCGCGCACCGTCGACT |
| sequence | | |
| Fourth stuffer | 12 | CTCGACTAGAGCATGGCTACGTAGATAAGT |
| sequence | | AGCATGGGGGGATTTAAATTAGGGATAACA |
| | | GGGTAATGGCGCGGGCCGC |
| Fifth stuffer | 13 | GGCGCCTGATGCGGTATTTTCTCCTTACGCATCTGT |
| sequence | | GCGGTATTTCACACCGCATACGTCAAAGCAACCATA |
| | | GTACGCGCCCTGTAGCGGCGCATTAAGCGCGGCGG |
| | | GTGTGGTGGTTACGCGCAGCGTGACCGCTACACTTG |
| | | CCAGCGCCCTAGCGCCCGCTCCTTTCGCTTT |
| | | CTTCCCTTCCTTTCTCGCCACGTTCGCCGGC |
| | | TTTCCCCGTCAAGCTCTAAATCGGGGGCTCC |
| | | CTTTAGGGTTCCGATTTAGTGCTTTACGGCAC |
| | | CTCGACCCCAAAAAACTTGATTTGGGTGATGG |
| | | TTCACGTAGTGCAATTGTATCTAAGCCGTGTAA |
| | | TGAGAACATCCACACCTTAGTGAATCGATGCGC |
| | | CGCTTCGGAATACCGTTTTGGCTACCTGTTACTA |
| | | AGCCCATCGCGATTTTCAGGTATCGTGCACGTAG |
| | | GGTTGCACCGCACGCATGTCGAACTGGTGGCGA |
| | | AGTACGATTCCACGACCGGTGTACGATTCAACTA |
| | | CGCGGACGTGACGAGCTTCTTATATATGCTTCGC |
| | | CCGCCGGACCGGCCTCGTGATGGGGTAGCTGCG |
| | | CATGAGCTTATGACAAATAACGAGTGTGTACTTGT |
| | | TTAATCATCTCACAGTGAAAGTCGGGAGAATAGGA |
| | | GCCGCTACACACATTTTACCGCAACTACACCTAAC |
| | | TGAGATACTACCATAGATGACTACCCATCCCTCTA |
| | | GGCCTTAGATAACCGGATACAGTGACTTTGATAGG |
| | | TTTGTGGGGTACAGCTATGACTTGCATAGCTGCGT |
| | | ATGGAGGAAGGAACTTTTGCGTGTTAGTATGTTGA |
| | | CCCGTGTATTACGCATGGGGGTAGATCATGTGGGT |
| | | AGAGACATCCAGGTCAAGTTCTCGACCTTCTCGTG |
| | | GGAGGTGATCCAGTTCACTATAGGACCATTCCATT |
| | | CGAGCATGGCACTAAGTACGCCGTCCCCATTCTGG |
| | | TAATCATCATCCCTATCAGGGTTTGGAGTGAGTGGT |
| | | GACGGTTATTCCCCAGGAATGGACTTCTTATTCACA |
| | | GTCGGTCACATTGGGCTACTCCTTGGGTCTTCCGCT |
| | | TGGCCCGGTCTGTTGGGCCGCCATTGCGTGAGTTTC |
| | | GGCCCTGTGCTGCGCTGTGTCGGCGATTCTCATTGG |
| | | GGCCTCACATCTGGAAACCCCGACCTATTTTGACGGC |
| | | ACCATTGGCGGAAGTTGCTGGGCTTGTGCACCGTGAA |
| | | GTCCTCCGCGGGCCTCCCCCCTACAAAAGACGATAAG |
| | | CTCCGGCAAGCAGCATTGATCAACGCAAGGATCGGCG |
| | | ATATTAACAGAGATACGGCTGATTACTCTTGTTGGTGTG |
| | | GTATCGCTAAACTGCGCCGCGGAGCCTTATGGCAAAGT |
| | | CGTCCGCGAACCATTCCGGTAGCGCTTAAGGTCCATAG |
| | | CACATTCATCGCATCCCGGCGTGCGTTCAATTTGACGAC |
| | | CCCTTGGCGCAAAGGTGCTGGCCACGTGCTAAATTAAAG |
| | | CGGCTGCACTGCTGTAAGGTCCGTTACGGAGAGGGGGG |
| | | CCTGGGGAGAGCACTAGCCCATCGACCTGTACAGGAACA |
| | | CTCTATATTGTTCTCGGACGGACAGATTACTAGAGTGCCG |
| | | CTTTCAGCCCCCCTGTCGTCGCCGACGTCTGTAATATGGC |
| | | GTTGTTGTGATTCCACTCTATTGAGGCATTA |
| | | ACTGATGCGTAAGGAGATCTGGAATGAACTGG |
| | | CCTATGTCACTGAAACTGTGCAAACACCCAATG |
| | | TCGTTAGTGTAGGTTCTGACCGATACCTGCTTC |
| | | GTTGAGAACTCACAATTTTACAACTGGGGACAT |
| | | AATCCCTACGCCCATCATCTACTCGCGTCCCTG |
| | | TGGCTCCAGTTCATGTAATGGGAGAGTATCCTC |
| | | CACAAGATCTAGTGCTATGGTGGTATAGTAAGC |
| | | TCGTACTGTGGTAGACGCGACACGGGTAGGAC |
| | | CATCAGTAATAAGGATAGTGCGAAAGCTCACAG |
| | | ACCACTGCCTATAGGGGGTGCTTACTTTTACAAA |
| | | AAGCGACTGTCAGTATAACCCCACGAGGATTCG |
| | | AAAAGGTGAACCGACCCAGACGATCCGGAGGCA |
| | | CGGGCCTCAAAGCCGCGACACGACGGCTGTCGG |
| | | CCGGTAACAGTAACCCCGGAGTGAACTCCTATGG |
| | | GGCTGGATAGAACAGCCCTGGTGGGCCCCATCAG |
| | | CAACCCGAATACGTGGCTTTTCGGGAGGCGGCCG |
| | | GAGGGGCGATGTCTTCCACTATTCGAGGCCGTTCG |
| | | TTAATACTTGTTGCGTTCCTAGCCGCTATATTTGTCT |
| | | CTTTGCCGACTAATGTGGACAAGCACACCATAGCCA |
| | | TTTATCGGAGCGCCTCGGAATACGGTATGAGCAGG |
| | | CGCCTCGTGAGACCATTGCGAATACCAGGTGTCCT |
| | | GTGGCGCCTGATGCGGTATTTTCTCCTTACGCATC |
| | | TGTGCGGTATTTCACACCGCATACGTCAAAGCAAC |
| | | CATAGTACGCGCCCTGTAGCGGCGCATTAAGCGCG |
| | | GCGGGTGTGGTGGTTACGCGCAGCGTGACCGCTAC |
| | | ACTTGCCAGCGCCCTAGCGCCCGCTCCTTTCGCTTT |
| | | CTTCCCTTCCTTTCTCGCCACGTTCGCCGGCTTTCCC |
| | | CGTCAAGCTCTAAATCGGGGGCTCCCTTTAGGGTTCC |
| | | GATTTAGTGCTTTACGGCACCTCGACCCCAAAAAACTT |
| | | GATTTGGGTGATGGTTCACGTAGTGGGCCATCGCCCT |
| | | GATAGACGGTTTTTCGCCCTTTGACGTTGGAGTCCACG |
| | | TTCTTTAATAGTGGACTCTTGTTCCAAACTGGAACAACA |
| | | CTCAACCCTATCTCGGGCTATTCTTTTGATTTATAAGGG |
| | | ATTTTGCCGATTTCGGCCTATTGGTTAAAAAATGAGCTG |
| | | ATTTAACAAAAATTTAACGCGAATTTTAACAAAATATTAA |
| | | CGTTTACAATTTTATGGTGCACTCTCAGTACAATCTGCTC |
| | | TGATGCCGCATAGTTAAGCCAGCCCCGACACCCGCCAA |
| | | CACCCGCTGACGCGCCCTGACGGGCTTGTCTGCTCCCG |
| | | GCATCCGCTTACAGACAAGCTGTGACCGTCTCCGGGAG |
| | | CTGCATGTGTCAGAGGTTTTCACCGTCATCACCGAAACG |
| | | CGCGAGACGAAAGGGCCTCGTGATACGCCTATTTTTATA |
| | | GGTTAATGTCATGATAATAATGGTTTCTTAGACGTCAGGT |
| | | GGCACTTTTCGGGGAAATGTGCGCGGAACCCCTATTTG |
| | | TTTATTTTTCTAAATACATTCAAATATGTATCCGCTCATGA |
| | | GACAATAACCCTGATAAATGCTTCAATAATATTGAAAAA |
| | | GGAAGAGT |
| Sixth stuffer | 14 | TAACTGTCAGACCAAGTTTACTCATATATACTTTAGATTGA |
| sequence | | TTTAAAACTTCATTTTTAATTTAAAAGGATCTAGGTGAAGA |
| | | TCCTTTTTGATAATCTCATGACCAAAATCCCTTAACGTGAG |
| | | TTTTCGTTCCACTGAGCGTCAGACCCC |
| Seventh stuffer | 15 | CATGTACATGT |
| sequence | | |
| Kanamycin | 16 | ATGAGCCATATTCAACGGGAAACGTCTTGCTC |
| resistance gene | | GAGGCCGCGATTAAATTCCAACATGGATGCTGA |
| | | TTTATATGGGTATAAATGGGCTCGCGATAATGTC |
| | | GGGCAATCAGGTGCGACAATCTATCGATTGTATG |
| | | GGAAGCCCGATGCGCCAGAGTTGTTTCTGAAACA |
| | | TGGCAAAGGTAGCGTTGCCAATGATGTTACAGATG |
| | | AGATGGTCAGACTAAACTGGCTGACGGAATTTATG |
| | | CCTCTTCCGACCATCAAGCATTTTATCCGTACTCCT |
| | | GATGATGCATGGTTACTCACCACTGCGATCCCCGG |
| | | GAAAACAGCATTCCAGGTATTAGAAGAATATCCTGAT |
| | | TCAGGTGAAAATATTGTTGATGCGCTGGCAGTGTTCC |
| | | TGCGCCGGTTGCATTCGATTCCTGTTTGTAATTGTCC |
| | | TTTTAACAGCGATCGCGTATTTCGTCTCGCTCAGGCG |
| | | CAATCACGAATGAATAACGGTTTGGTTGATGCGAGTG |
| | | ATTTTGATGACGAGCGTAATGGCTGGCCTGTTGAACA |
| | | AGTCTGGAAAGAAATGCATAAGCTTTTGCCATTCTCAC |
| | | CGGATTCAGTCGTCACTCATGGTGATTTCTCACTTGAT |
| | | AACCTTATTTTTGACGAGGGGAAATTAATAGGTTGTATT |
| | | GATGTTGGACGAGTCGGAATCGCAGACCGATACCAGG |
| | | ATCTTGCCATCCTATGGAACTGCCTCGGTGAGTTTTCTC |
| | | CTTCATTACAGAAACGGCTTTTTCAAAAATATGGTATTGA |
| | | TAATCCTGATATGAATAAATTGCAGTTTCATTTGATGCTC |
| | | GATGAGTTTTTCTAA |
| pUC origin | 17 | GTAGAAAAGATCAAAGGATCTTCTTGAGATCCTTTTTTT |
| | | CTGCGCGTAATCTGCTGCTTGCAAACAAAAAAACCACC |
| | | GCTACCAGCGGTGGTTTGTTTGCCGGATCAAGAGCTAC |
| | | CAACTCTTTTTCCGAAGGTAACTGGCTTCAGCAGAGCGC |
| | | AGATACCAAATACTGTCCTTCTAGTGTAGCCGTAGTTAG |
| | | GCCACCACTTCAAGAACTCTGTAGCACCGCCTACATACC |
| | | TCGCTCTGCTAATCCTGTTACCAGTGGCTGCTGCCAGTG |
| | | GCGATAAGTCGTGTCTTACCGGGTTGGACTCAAGACGAT |
| | | AGTTACCGGATAAGGCGCAGCGGTCGGGCTGAACGGGG |
| | | GGTTCGTGCACACAGCCCAGCTTGGAGCGAACGACCTAC |
| | | ACCGAACTGAGATACCTACAGCGTGAGCTATGAGAAAGC |
| | | GCCACGCTTCCCGAAGGGAGAAAGGCGGACAGGTATCCG |
| | | GTAAGCGGCAGGGTCGGAACAGGAGAGCGCACGAGGGA |
| | | GCTTCCAGGGGGAAACGCCTGGTATCTTTATAGTCCTGTC |
| | | GGGTTTCGCCACCTCTGACTTGAGCGTCGATTTTTGTGAT |
| | | GCTCGTCAGGGGGGCGGAGCCTATGGAAAAACGCCAGCA |
| | | ACGCGGCCTTTTTACGGTTCCTGGCCTTTTGCTGGCCTT |
| | | TTGCTCA |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

For purposes of interpreting this specification, terms used in the singular will also include the plural and vice versa.

Currently there is no appropriate human cone photoreceptor cell lines available, which represent a bottleneck in the study of retinal diseases. Thus, it is critical to develop good in vitro models for human cone photoreceptors for the retinal research field, as some questions regarding the unique nature of the human visual system cannot be answered by animal models. This research described herein is the development of direct reprogramming method to generate human cone photoreceptors in vitro. Critically, the direct reprogramming method is much faster (˜2 weeks) compared to iPSC generation and differentiation (˜3-6 months), making it more cost-effective for generating human cone photoreceptors in vitro. The derived human cone photoreceptors will provide both a better in vitro model to study retinal biology and diseases, providing a platform for drug testing in a clinically relevant cell type, as well as a cellular source for tissue engineering and transplantation for cell therapy.

Further, the invention described herein also includes the in vivo reprogramming of cells to photoreceptor cells, directly demonstrating an in vivo gene therapy application. In particular, the inventors show use of a gene therapy approach to prevent vision loss in a rat photoreceptor degeneration model. P23H is a well-established rat model for retinitis pigmentosa caused by a rhodopsin mutation, which undergoes a gradual photoreceptor loss characteristic of human autosomal dominant retinitis pigmentosa. The inventors performed viral delivery of a representative set of transcription factors by subretinal injection into P23H rats, and analysed visual function using electroretinogram (ERG) before and after treatment for 4 weeks. The results shown in the Examples that P23H rats treated with a representative set of factors prevent progressive loss after 4 weeks compared to untreated controls providing direct supporting evidence for the therapeutic potential of using various transcription factors describe herein to prevent vision loss in vivo.

In one aspect, the present invention provides compositions and methods for direct reprogramming or transdifferentiation of source cells to target cells, without the source cell becoming an induced pluripotent stem cell (iPS) intermediately prior to becoming a target cell. In comparison to iPS cell technology, transdifferentiation is highly efficient and poses a very low risk of teratoma formation for downstream applications.

The process of reprogramming a cell alters the type of progeny a cell can produce and includes transdifferentiation. Transdifferentiation of one somatic cell provides a cell exhibiting at least one characteristic of another somatic cell type.

A source cell may be any cell type described herein, including a somatic cell or a diseased somatic cell. The somatic cell may be an adult cell or a cell derived from an adult. The diseased cell may be a cell displaying one or more detectable characteristics of a disease or condition, for example the diseased cell may be a cancer cell displaying one or more clinical or biochemical markers of a cancer. Examples of source cells include glial cells, such as a Müller glial (MG) cell, an astrocyte and a microglial cell.

As used herein, the term “somatic cell” refers to any cell forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. The somatic cells may be immortalized to provide an unlimited supply of cells, for example, by increasing the level of telomerase reverse transcriptase (TERT). For example, the level of TERT can be increased by increasing the transcription of TERT from the endogenous gene, or by introducing a transgene through any gene delivery method or system.

Unless otherwise indicated the methods for reprogramming somatic cells can be performed in vitro or in vivo, where in vitro is practiced using isolated somatic cells maintained in culture.

Suitable somatic cells are receptive, or can be made receptive using methods generally known in the scientific literature, to uptake of transcription factors including genetic material encoding the transcription factors. Uptake-enhancing methods can vary depending on the cell type and expression system. Exemplary conditions used to prepare receptive somatic cells having suitable transduction efficiency are well-known by those of ordinary skill in the art.

By a “cone cell”, also referred to herein as a “cone photoreceptor” or “cone”, it is meant the subtype of photoreceptor cells in the retina of the eye that function best in relatively bright light. Cones are sensitive to specific wavelengths of light and hence support the perception of colour. In addition, cones respond faster to stimuli than rod photoreceptors, perceiving finer detail and more rapid changes in images than rods, and hence, support high acuity vision for activities where visual detail is of primary importance such as reading and driving. Cones are readily identifiable in cross-sections of the retina by the cone-like shape of their outer segments. They are also readily identifiable by their location in the retina, the highest density of cones existing at the 1.5 mm depression located in the centre of the macula of the retina, called the “fovea centralis” or “foveal pit”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein, refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of target cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not target cells or their progeny as defined by the terms herein.

A source cell is determined to be converted to a target cell, or become a target-like cell, by a method of the invention when it displays at least one characteristic of the target cell type, i.e. a cone photoreceptor cell. For example, a human Müller glial will be identified as converted to a cone photoreceptor-like cell, when a cell displays at least one characteristic of the cone photoreceptor cell type. Typically, a cell will display 1, 2, 3, 4, 5, 6, 7, 8 or more characteristics (or markers) of a cone photoreceptor cell. For example, where the target cell is a cone photoreceptor cell, a cell is identified or determined to be a cone photoreceptor-like cell when up-regulation of, or presence of, any one or more photoreceptor cell markers and/or change in cell morphology is detectable, preferably, the increase in opsin mRNA expression. Other examples of photoreceptor markers include ARR3, CNGB3, GNAT2, GNGT2, GRK7, GUCA1C, PDE6C, PDE6H, RXRG, THRB, OPN1LW, OPN1MW and OPN1SW, an electrophysiological response in a photopic condition, for example, as described in the Examples. Additional examples of cone photoreceptor markers include the opsins that are light-detecting molecules. For example, red/green opsin (cone photoreceptor cells), blue opsin (cone photoreceptor cells), and recoverins (rod photoreceptor cells, cone photoreceptor cells). In any aspect of the invention, the target cell characteristic may be determined by analysis of cell morphology, gene expression profiles, activity assay, protein expression profile, surface marker profile, or differentiation ability. Examples of characteristics or markers include those that are described herein and those known to the skilled person.

The transcription factors referred to herein are referred to by the HUGO Gene Nomenclature Committee (HGNC) Symbol. Exemplary nucleotide sequences for each transcription factor are shown in Table 1 below. The nucleotide sequences are derived from the Ensembl database (Flicek et al. (2014). Nucleic Acids Research Volume 42, Issue D1. Pp. D749-D755) version 83. Also contemplated for use in the invention is any homolog, ortholog or paralog of a transcription factor referred to herein.

The skilled person will appreciate that this information may be used in performing the methods of the present invention, for example, for the purposes of providing increased amounts of transcription factors in source cells, or providing nucleic acids or the like for recombinantly expressing a transcription factor in a source cell.

TABLE 1
Accession numbers identifying nucleotide sequences and amino acid
sequences of transcription factors and proteins referred to herein.

| Transcription factor | | | Uniprot |
|---|---|---|---|
| Associated Gene Name | Abbreviation | Ensembl Gene ID | ID |
| CRX | C | ENSG00000105392 | O43186 |
| MEF2C | M | ENSG00000081189 | Q06413 |
| THRB | T | ENSG00000151090 | P10828 |
| RAX | R | ENSG00000134438 | Q9Y2V3 |
| NEUROD1 | N | ENSG00000162992 | Q13562 |
| RORA | Roa | ENSG00000069667 | P35398 |
| OTX2 | O | ENSG00000165588 | P32243 |
| NEUROG2 / NGN2 | Ng | ENSG00000178403 | Q9H2A3 |
| PAX6 | P | ENSG00000007372 | P26367 |
| FOXP1 | F | ENSG00000114861 | Q9H334 |
| ASCL1 | A | ENSG00000139352 | P50553 |
| ONECUT1 | On | ENSG00000169856 | Q9UBC0 |

The term a “variant” refers to a polypeptide that is at least 70%, 80%, 85%, 90%, 95%, 98%, or 99% identical to the full length polypeptide. The present invention contemplates the use of variants of the transcription factors described herein, including the sequences listed in Table 1. The variant could be a fragment of full length polypeptide or a naturally occurring splice variant. The variant could be a polypeptide at least 70%, 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof has a functional activity of interest such as the ability to promote conversion of a source cell type to a target cell type. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein. One of skill in the art will be aware of, or will readily be able to ascertain, whether a particular polypeptide variant, fragment, or derivative is functional using assays known in the art. For example, the ability of a variant of a transcription factor to convert a source cell to a target cell type can be assessed using the assays as disclose herein in the Examples. Other convenient assays include measuring the ability to activate transcription of a reporter construct containing a transcription factor binding site operably linked to a nucleic acid sequence encoding a detectable marker such as luciferase. In certain embodiments of the invention a functional variant or fragment has at least 50%, 60%, 70%, 80%, 90%, 95% or more of the activity of the full length wild type polypeptide.

As used herein, the terms “biological activity” and “biologically active” refer to the activity attributed to a particular biological element in a cell. For example, the biological activity of a polypeptide or functional fragment or variant thereof refers to the ability of the polypeptide or functional fragment or variant thereof to carry out its native functions of, e.g., binding, enzymatic activity, etc. For example, a biologically active fragment or variant of a transcription factor retains the ability to bind to DNA and regulate transcription. Typically, the biologically active fragment or variant of regulates transcription to a level at least 70%, 75%, 80%, 85%, 90% or 95% of wildtype protein, preferably human.

Further, the biological activity of a gene regulatory element, e.g. promoter, enhancer, Kozak sequence, and the like, refers to the ability of the regulatory element or functional fragment or variant thereof to regulate, i.e. promote, enhance, or activate the translation of, respectively, the expression of the gene to which it is operably linked.

The term “increasing the amount of” with respect to increasing an amount of a transcription factor, refers to increasing the quantity of the transcription factor in a cell of interest (e.g., a source cell such as a fibroblast or keratinocyte cell). In some embodiments, the amount of transcription factor is “increased” in a cell of interest (e.g., a cell into which an expression cassette directing expression of a polynucleotide encoding one or more transcription factors has been introduced) when the quantity of transcription factor is at least 10%, 20%, 30%, 40%, 50%, 60%, 70%, 80%, 90%, or more relative to a control (e.g., a glial cell into which none of said expression cassettes have been introduced). However, any method of increasing an amount of a transcription factor is contemplated including any method that increases the amount, rate or efficiency of transcription, translation, stability or activity of a transcription factor (or the pre-mRNA or mRNA encoding it).

In particularly preferred embodiments, the method may include use of a CRISPR activation system (CRISPRa), or variations thereof, for activating/increasing the expression of endogenous genes in the source cell and encoding the transcription factors for which an increased amount is desired, so as to facilitate reprogramming. Such methods are well known to a person skilled in the art, such as those published in Fang et al. Molecular therapy. Nucleic Acids, 20 Nov. 2018, 14:184-191, incorporated herein by reference.

In addition, down-regulation or interference of a negative regulator of transcription expression, increasing efficiency of existing transcription (e.g. SINEUP) are also considered.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. A system or set of components, such as a CRISPR activation system, for example as described herein, is also contemplated as an agent.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from that of natural cells, or is otherwise flanked by a different nucleic acid sequence than that found in nature. An exogenous nucleic acid may also be extra-chromosomal, such as an episomal vector.

The methods of the invention include high-throughput screening applications. For example, a high-throughput screening assay may be used which comprises any of the assays according to the invention wherein aliquots of a system that allows the product or expression of a transcription factor are exposed to a plurality of candidate agents within different wells of a multi-well plate. Further, a high-throughput screening assay according to the disclosure involves aliquots of a system that allows the product or expression of a transcription factor which are exposed to a plurality of candidate agents in a miniaturized assay system of any kind.

The method of the disclosure may be “miniaturized” in an assay system through any acceptable method of miniaturization, including but not limited to multi-well plates, such as 24, 48, 96 or 384-wells per plate, microchips or slides. The assay may be reduced in size to be conducted on a micro-chip support, advantageously involving smaller amounts of reagent and other materials. Any miniaturization of the process which is conducive to high-throughput screening is within the scope of the invention.

In any method of the invention the target cells can be transferred into the same mammal from which the source cells were obtained. In other words, the source cells used in a method of the invention can be an autologous cell, i.e., can be obtained from the same individual in which the target cells are to be administered. Alternatively, the target cell can be allogenically transferred into another individual. Preferably, the cell is autologous to the subject in a method of treating or preventing a medical condition in the individual.

As used herein, “culturing” relates to contacting cells with a cell culture medium, typically for a sufficient time and under conditions to allow cell differentiation or proliferation. The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. Exemplary cell culture medium for use in methods of the invention are shown in Table 2.

TABLE 2
Cell culture media that can be used to culture various cell types,
referred to herein as MG cell media and Photoreceptor cell media

| Cell | Media | Cat#: | Company |
|---|---|---|---|
| MG cells | DMEM | 11995-073 | All from |
| | 10% fetal bovine | 26140079 | Thermo |
| | serum | 35050061 | scientific |
| | 2 mM glutamax | 15140122 | |
| | 0.5% Penicillin- | | |
| | streptomycin | | |
| Photoreceptor cells | Neurobasal | 21103049 | Thermo |
| | medium | | scientific |
| | B27 (1x) | 17504-044 | Sigma-Aldrich |
| | T3 | T2877 | |

A nucleic acid or vector comprising a nucleic acid as described herein may include one or more of the sequences referred to above in Table 1 or a sequence encoding any one or more of the amino acid sequences listed in Table 1.

The term “expression” refers to the cellular processes involved in producing RNA and proteins and as appropriate, secreting proteins, including where applicable, but not limited to, for example, transcription, translation, folding, modification and processing.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “vector” refers to a carrier DNA molecule into which a DNA sequence can be inserted for introduction into a host or source cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “AAV” is an abbreviation for adeno-associated virus, and may be used to refer to the virus itself or derivatives thereof. The term covers all subtypes and both naturally occurring and recombinant forms, except where required otherwise. The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e. a polynucleotide other than a wild-type AAV genome such as a transgene to be delivered to a mammalian cell), it is typically referred to as a “rAAV vector particle” or simply a “rAAV vector”. Thus, production of rAAV particle necessarily includes production of rAAV vector, as such a vector is contained within a rAAV particle.

The term “replication defective” as used herein relative to an AAV viral vector of the invention means the AAV vector cannot independently replicate and package its genome. For example, when a cell of a subject is infected with rAAV virions, the heterologous gene is expressed in the infected cells, however, due to the fact that the infected cells lack AAV rep and cap genes and accessory function genes, the rAAV is not able to replicate further.

An “AAV variant” or “AAV mutant” as used herein refers to a viral particle composed of: a) a variant AAV capsid protein, where the variant AAV capsid protein comprises at least one amino acid difference (e.g., amino acid substitution, amino acid insertion, amino acid deletion) relative to a corresponding parental AAV capsid protein, and where the variant capsid protein confers increased infectivity of a retinal cell compared to the infectivity of the retinal cell by an AAV virion comprising the corresponding parental AAV capsid protein, where the AAV capsid protein does not comprise an amino acid sequence present in a naturally occurring AAV capsid protein; and b) a heterologous nucleic acid comprising a nucleotide sequence encoding a heterologous gene product.

The abbreviation “rAAV” refers to recombinant adeno-associated virus, also referred to as a recombinant AAV vector (or “rAAV vector”). A “rAAV vector” as used herein refers to an AAV vector comprising a polynucleotide sequence not of AAV origin (i.e., a polynucleotide heterologous to AAV), typically a sequence of interest for the genetic transformation of a cell, e.g. a transgene as described herein. In general, the heterologous polynucleotide is flanked by at least one, and generally by two AAV inverted terminal repeat sequences (ITRs). The term rAAV vector encompasses both rAAV vector particles and rAAV vector plasmids.

As used herein, a transgene is a gene (e.g. DNA or RNA, preferably mRNA) that is delivered to a cell by a vector.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g. ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

As used herein, the term “adenovirus” refers to a virus of the family Adenovirida. Adenoviruses are medium-sized (90-100 nm), nonenveloped (naked) icosahedral viruses composed of a nucleocapsid and a double-stranded linear DNA genome.

As used herein, the term “non-integrating viral vector” refers to a viral vector that does not integrate into the host genome; the expression of the gene delivered by the viral vector is temporary. Since there is little to no integration into the host genome, non-integrating viral vectors have the advantage of not producing DNA mutations by inserting at a random point in the genome. For example, a non-integrating viral vector remains extra-chromosomal and does not insert its genes into the host genome, potentially disrupting the expression of endogenous genes. Non-integrating viral vectors can include, but are not limited to, the following: adenovirus, alphavirus, picornavirus, and vaccinia virus. These viral vectors are “non-integrating” viral vectors as the term is used herein, despite the possibility that any of them may, in some rare circumstances, integrate viral nucleic acid into a host cell's genome. What is critical is that the viral vectors used in the methods described herein do not, as a rule or as a primary part of their life cycle under the conditions employed, integrate their nucleic acid into a host cell's genome.

The vectors described herein can be constructed and engineered using methods generally known in the scientific literature to increase their safety for use in therapy, to include selection and enrichment markers, if desired, and to optimize expression of nucleotide sequences contained thereon. The vectors should include structural components that permit the vector to self-replicate in the source cell type. For example, the known Epstein Barr oriP/Nuclear Antigen-1 (EBNA-I) combination (see, e.g., Lindner, S. E. and B. Sugden, The plasmid replicon of Epstein-Barr virus: mechanistic insights into efficient, licensed, extrachromosomal replication in human cells, Plasmid 58:1 (2007), incorporated by reference as if set forth herein in its entirety) is sufficient to support vector self-replication and other combinations known to function in mammalian, particularly primate, cells can also be employed. Standard techniques for the construction of expression vectors suitable for use in the present invention are well-known to one of ordinary skill in the art and can be found in publications such as Sambrook J, et al., “Molecular cloning: a laboratory manual,” (3rd ed. Cold Spring harbor Press, Cold Spring Harbor, N. Y. 2001), incorporated herein by reference as if set forth in its entirety.

In the methods of the invention, genetic material encoding the relevant transcription factors required for a conversion is delivered into the source cells via one or more reprogramming vectors. Each transcription factor can be introduced into the source cells as a polynucleotide transgene that encodes the transcription factor operably linked to a heterologous promoter that can drive expression of the polynucleotide in the source cell.

Suitable reprogramming vectors are any described herein, including episomal vectors, such as plasmids, that do not encode all or part of a viral genome sufficient to give rise to an infectious or replication-competent virus, although the vectors can contain structural elements obtained from one or more virus. One or a plurality of reprogramming vectors can be introduced into a single source cell. One or more transgenes can be provided on a single reprogramming vector. One strong, constitutive transcriptional promoter can provide transcriptional control for a plurality of transgenes, which can be provided as an expression cassette. Separate expression cassettes on a vector can be under the transcriptional control of separate strong, constitutive promoters, which can be copies of the same promoter or can be distinct promoters. Various heterologous promoters are known in the art and can be used depending on factors such as the desired expression level of the transcription factor. It can be advantageous, as exemplified below, to control transcription of separate expression cassettes using distinct promoters having distinct strengths in the source cells. Another consideration in selection of the transcriptional promoters is the rate at which the promoter(s) is silenced. The skilled artisan will appreciate that it can be advantageous to reduce expression of one or more transgenes or transgene expression cassettes after the product of the gene(s) has completed or substantially completed its role in the reprogramming method. Exemplary promoters are the human EF1a elongation factor promoter, CMV cytomegalovirus immediate early promoter and CAG chicken albumin promoter, and corresponding homologous promoters from other species. In human somatic cells, both EF1a and CMV are strong promoters, but the CMV promoter is silenced more efficiently than the EF1a promoter such that expression of transgenes under control of the former is turned off sooner than that of transgenes under control of the latter. The transcription factors can be expressed in the source cells in a relative ratio that can be varied to modulate reprogramming efficiency. Preferably, where a plurality of transgenes is encoded on a single transcript, an internal ribosome entry site is provided upstream of transgene(s) distal from the transcriptional promoter. Although the relative ratio of factors can vary depending upon the factors delivered, one of ordinary skill in possession of this disclosure can determine an optimal ratio of factors.

The skilled artisan will appreciate that the advantageous efficiency of introducing all factors via a single vector rather than via a plurality of vectors, but that as total vector size increases, it becomes increasingly difficult to introduce the vector. The skilled artisan will also appreciate that position of a transcription factor on a vector can affect its temporal expression, and the resulting reprogramming efficiency. As such, Applicants employed various combinations of factors on combinations of vectors. Several such combinations are here shown to support reprogramming.

After introduction of the reprogramming vector(s) and while the source cells are being reprogrammed, the vectors can persist in target cells while the introduced transgenes are transcribed and translated. Transgene expression can be advantageously downregulated or turned off in cells that have been reprogrammed to a target cell type. The reprogramming vector(s) can remain extra-chromosomal. At extremely low efficiency, the vector(s) can integrate into the cell's genome. The examples that follow are intended to illustrate but in no way limit the present invention.

Suitable methods for nucleic acid delivery for transformation of a cell for use with the current invention are believed to include virtually any method by which a nucleic acid (e.g., DNA) can be introduced into a cell as described herein or as would be known to one of ordinary skill in the art (e.g., Stadtfeld and Hochedlinger, Nature Methods 6 (5): 329-330 (2009); Yusa et al., Nat. Methods 6:363-369 (2009); Woltjen, et al., Nature 458, 766-770 (9 Apr. 2009)). Such methods include, but are not limited to, direct delivery of DNA such as by ex vivo transfection (Wilson et al., Science, 244:1344-1346, 1989, Nabel and Baltimore, Nature 326:711-713, 1987), optionally with a lipid-based transfection reagent such as Fugene6 (Roche) or Lipofectamine (Invitrogen), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van Der Eb, Virology, 52:456-467, 1973; Chen and Okayama, Mol. Cell Biol., 7 (8): 2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., J Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); and any combination of such methods, each of which is incorporated herein by reference.

A number of polypeptides capable of mediating introduction of associated molecules into a cell have been described previously and can be adapted to the present invention. See, e.g., Langel (2002) Cell Penetrating Peptides: Processes and Applications, CRC Press, Pharmacology and Toxicology Series. Examples of polypeptide sequences that enhance transport across membranes include, but are not limited to, the Drosophila homeoprotein antennapedia transcription protein (AntHD) (Joliot et al., New Biol. 3:1121-34, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP22 (Elliott and O'Hare, Cell 88:223-33, 1997); the HIV-1 transcriptional activator TAT protein (Green and Loewenstein, Cell 55:1179-1188, 1988; Frankel and Pabo, Cell 55:1 289-1193, 1988); Kaposi FGF signal sequence (kFGF); protein transduction domain-4 (PTD4); Penetratin, M918, Transportan-10; a nuclear localization sequence, a PEP-I peptide; an amphipathic peptide (e.g., an MPG peptide); delivery enhancing transporters such as described in U.S. Pat. No. 6,730,293 (including but not limited to an peptide sequence comprising at least 5-25 or more contiguous arginines or 5-25 or more arginines in a contiguous set of 30, 40, or 50 amino acids; including but not limited to an peptide having sufficient, e.g., at least 5, guanidino or amidino moieties); and commercially available Penetratin™ 1 peptide, and the Diatos Peptide Vectors (“DPVs”) of the Vectocell® platform available from Daitos S. A. of Paris, France. See also, WO/2005/084158 and WO/2007/123667 and additional transporters described therein. Not only can these proteins pass through the plasma membrane but the attachment of other proteins, such as the transcription factors described herein, is sufficient to stimulate the cellular uptake of these complexes.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific (such as glial cell-specific), tissue-specific, or species specific. Promoters may “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

An “enhancer” as used herein encompasses a cis-acting element that stimulates or inhibits transcription of adjacent genes. An enhancer that inhibits transcription also is termed a “silencer”. Enhancers can function (i.e., can be associated with a coding sequence) in either orientation, over distances of up to several kilobase pairs (kb) from the coding sequence and from a position downstream of a transcribed region.

A “termination signal sequence” as used herein encompasses any genetic element that causes RNA polymerase to terminate transcription, such as for example a polyadenylation signal sequence.

A “polyadenylation signal sequence” as used herein encompasses a recognition region necessary for endonuclease cleavage of an RNA transcript that is followed by the polyadenylation consensus sequence AATAAA. A polyadenylation signal sequence provides a “polyA site”, i.e. a site on a RNA transcript to which adenine residues will be added by post-transcriptional polyadenylation.

### Gene Therapy Vectors

Any convenient vector, such as a gene therapy vector or gene delivery vector (used interchangeably herein) that finds use delivering nucleic acids or nucleotide sequences as described herein to cells in the retina is encompassed by the vectors of the present disclosure. For example, the vector may comprise single or double stranded nucleic acid, e.g. single stranded or double stranded DNA or RNA. For example, the gene delivery vector may be a naked DNA or RNA, e.g. a plasmid, a minicircle, etc. As another example, the gene delivery vector may be a virus, e.g. an adenovirus, an adeno-associated virus (AAV), baculovirus or a retrovirus, e.g. Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) or lentivirus. While embodiments encompassing the use of adeno-associated virus are described in greater detail below, it is expected that the ordinarily skilled artisan will appreciate that similar knowledge and skill in the art can be brought to bear on non-AAV gene therapy vectors as well. See, for example, the discussion of retroviral vectors in, e.g., U.S. Pat. Nos. 7,585,676 and 8,900,858, and the discussion of adenoviral vectors in, e.g. U.S. Pat. No. 7,858,367, the full disclosures of which are incorporated herein by reference.

Gene therapy vectors, e.g. rAAV, lentivirus and baculovirus, virions encapsulating the polynucleotide cassettes of the present disclosure, may be produced using standard methodology. In some embodiments, the gene delivery vector is a recombinant adeno-associated virus (rAAV). In such embodiments, the expression construct encoding a set of transcription factors descried herein at (a) to (jj), or biologically active fragments or variants thereof is flanked on the 5′ and 3′ ends by functional AAV inverted terminal repeat (ITR) sequences. By “functional AAV ITR sequences” is meant that the ITR sequences function as intended for the rescue, replication and packaging of the AAV virion. Hence, AAV ITRs for use in the gene delivery vectors of the invention need not have a wild-type nucleotide sequence, and may be altered by the insertion, deletion or substitution of nucleotides or the AAV ITRs may be derived from any of several AAV serotypes, e.g. AAV1, AAV2, AAV3, AAV4, AAVS, AAV6, AAV7, AAV8, AAV9, AAV10, ShH10 and ShH10Y. Preferred AAV vectors have the wild type REP and CAP genes deleted in whole or part, but retain functional flanking ITR sequences.

In such embodiments, the nucleic acid comprising an expression construct is encapsidated within an AAV capsid, which may be derived from any adeno-associated virus serotype, including without limitation, AAV1, AAV2, AAV3, AAV4, AAV5, AAV6, AAV7, AAV8, AAV9, AAV10, etc. For example, the AAV capsid may be a wild type, or native, capsid. Wild type AAV capsids of particular interest include AAV2, AAV5, and AAV9. However, as with the ITRs, the capsid need not have a wild-type nucleotide sequence, but rather may be altered by the insertion, deletion or substitution of nucleotides in the VP1, VP2 or VP3 sequence, so long as the capsid is able to transduce cone cells. Put another way, the AAV capsid may be a variant AAV capsid. Variant AAV capsids of particular interest include those comprising a peptide insertion within residues 580-600 of AAV2 or the corresponding residues in another AAV, e.g. LGETTRP, NETITRP, KAGQANN, KDPKTTN, KDTDTTR, RAGGSVG, AVDTTKF, or STGKVPN, as disclosed in US Application No. US 2014/0294771, the full disclosure of which is incorporated by reference herein. In some embodiments, the AAV vector is a “pseudotyped” AAV created by using the capsid (cap) gene of one AAV and the rep gene and ITRs from a different AAV, e.g. a pseudotyped AAV2 created by using rep from AAV2 and cap from AAV1, AAV3, AAV4, AAV5, AAV6, AAV7, AAV8, or AAV9 together with a plasmid containing a vector based on AAV2. For example, the AAV vector may be rAAV2/1, rAAV2/3, rAAV2/4, rAAV2/5, rAAV2/6, rAAV2/7, rAAV2/8, rAAV2/9, etc. Preferably, the rAAV is replication defective, in that the AAV vector cannot independently further replicate and package its genome. For example, when cone cells are transduced with rAAV virions, the gene is expressed in the transduced cone cells, however, due to the fact that the transduced cone cells lack AAV rep and cap genes and accessory function genes, the rAAV is not able to replicate.

In the case of rAAV virions, an AAV expression vector according to the invention may be introduced into a producer cell, followed by introduction of an AAV helper construct, where the helper construct includes AAV coding regions capable of being expressed in the producer cell and which complement AAV helper functions absent in the AAV vector. This is followed by introduction of helper virus and/or additional vectors into the producer cell, wherein the helper virus and/or additional vectors provide accessory functions capable of supporting efficient rAAV virus production. The producer cells are then cultured to produce rAAV.

In preparing the rAAV compositions, any host cells for producing rAAV virions may be employed, including, for example, mammalian cells (e.g. 293 cells), insect cells (e.g. SF9 cells), microorganisms and yeast. Host cells can also be packaging cells in which the AAV rep and cap genes are stably maintained in the host cell or producer cells in which the AAV vector genome is stably maintained and packaged. Exemplary packaging and producer cells are derived from SF-9, 293, A549 or Hela cells. AAV vectors are purified and formulated using standard techniques known in the art. These steps are carried out using standard methodology. Replication-defective AAV virions encapsulating the recombinant AAV vectors of the instant invention are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, expressly incorporated by reference herein in their entirety. Further compositions and methods for packaging are described in Wang et al. (US 2002/0168342), also incorporated by reference herein in its entirety.

Any suitable method for producing viral particles for delivery of the nucleic acids (e.g. DNA or RNA) or nucleotide sequences as described herein can be used, including but not limited to those described in the examples that follow. Any concentration of viral particles suitable to effectively transduce retinal cells can be prepared for contacting those cells in vitro or in vivo. For example, the viral particles may be formulated at a concentration of 10⁸ vector genomes per ml or more, for example, 5×10⁸ vector genomes per mL; 10⁹ vector genomes per mL; 5×10⁹ vector genomes per mL, 10¹⁰ vector genomes per mL, 5×10¹⁰ vector genomes per mL; 10¹¹ vector genomes per mL; 5×10¹¹ vector genomes per mL; 10¹² vector genomes per mL; 5×10¹² vector genomes per mL; 10¹³ vector genomes per mL; 1.5×10¹³ vector genomes per mL; 3×10¹³ vector genomes per mL; 5×10¹³ vector genomes per mL; 7.5×10¹³ vector genomes per mL; 9×10¹³ vector genomes per mL; 1×10¹⁴ vector genomes per mL, 5×10¹⁴ vector genomes per mL or more, but typically not more than 1×10¹⁵ vector genomes per mL. Similarly, any total number of viral particles suitable to provide appropriate transduction of retinal cells to confer the desired effect or treat the disease can be administered to the mammal or to the primate's eye. In various preferred embodiments, at least 10⁸; 5×10⁸; 10⁹; 5×10⁹; 10¹⁰, 5×10¹⁰, 10¹¹; 5×10¹¹; 10¹²; 10¹³; 5×10¹²; 10¹³; 1.5×10¹³; 3×10¹³; 5×10¹³; 7.5×10¹³; 9×10¹³; 1×10¹⁴ viral particles, or 5×10¹⁴ viral particles or more, but typically not more than 1×10¹⁵ viral particles are injected per eye. Any suitable number of administrations of the vector to the subject eye can be made. In one embodiment, the methods comprise a single administration; in other embodiments, multiple administrations are made over time as deemed appropriate by an attending clinician.

The vector may be formulated into any suitable unit dosage, including, without limitation, 1×10⁸ vector genomes or more, for example, 1×10⁹, 1×10¹⁰, 1×10¹¹, 1×10¹², or 1×10¹³ vector genomes or more, in certain instances, 1×10¹⁴ vector genomes, but usually no more than 4×10¹⁵ vector genomes. In some cases, the unit dosage is at most about 5×10¹⁵ vector genomes, e.g. 1×10¹⁴ vector genomes or less, for example 1×10¹³, 1×10¹², 1×10¹¹, 1×10¹⁰, or 1×10⁹ vector genomes or less, in certain instances 1×10⁸ vector genomes or less, and typically no less than 1×10⁸ vector genomes. In some cases, the unit dosage is 1×10¹⁰ to 1×10¹¹ vector genomes. In some cases, the unit dosage is 1×10¹⁰ to 3×10¹² vector genomes. In some cases, the unit dosage is 1×10⁹ to 3×10¹³ vector genomes. In some cases, the unit dosage is 1×10⁸ to 3×10¹⁴ vector genomes.

In some cases, the unit dosage of pharmaceutical composition may be measured using multiplicity of infection (MOI). By MOI it is meant the ratio, or multiple, of vector or viral genomes to the cells to which the nucleic acid may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI. In some cases, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some aspects, the amount of pharmaceutical composition comprises about 1×10⁸ to about 1×10¹⁵ recombinant viruses, about 1×10⁹ to about 1×10¹⁴ recombinant viruses, about 1×10¹⁰ to about 1×10¹³ recombinant viruses, or about 1×10¹¹ to about 3×10¹² recombinant viruses.

### Formulation

The nucleic acid or vector according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, liposome suspension or any other suitable form that may be administered to a person or animal in need of treatment. However, preferably the construct or vector is formulated for suitable administration to the subject's eye, preferably the retina, preferably by injection, more preferably by retinal injection (e.g subretinal injection), or most preferably by intravitreal injection. It will be appreciated that the vehicle of medicaments according to the invention should be one which is well-tolerated by the subject to whom it is given.

It will be appreciated that the amount of the nucleic acid or vector that is required is determined by its biological activity and bioavailability, which in turn depends on the mode of administration, the physiochemical properties of the nucleic acid or vector and whether it is being used as a monotherapy or in a combined therapy. The frequency of administration will also be influenced by the half-life of the construct or vector within the subject being treated. Optimal dosages to be administered may be determined by those skilled in the art, and will vary with the particular nucleic acid or vector in use, the strength of the pharmaceutical composition, the mode of administration, and the advancement of the retinal disorder. Additional factors depending on the particular subject being treated will result in a need to adjust dosages, including subject age, weight, gender, diet, and time of administration.

Generally, a daily dose of between 0.001 μg/kg of body weight and 10 mg/kg of body weight, or between 0.01 μg/kg of body weight and 1 mg/kg of body weight, of the nucleic acid or vector according to the invention may be used for treating, ameliorating, or preventing a retinal disorder, depending upon the nucleic acid or vector used.

The nucleic acid or vector may be administered before, during or after onset of the cone cell disorder. Daily doses may be given as a single administration (e.g. a single daily injection). Alternatively, the nucleic acid or vector may require administration twice or more times during a day. As an example, the nucleic acid or vector may be administered as two (or more depending upon the severity of the retinal disorder being treated) daily doses of between 0.07 ug and 700 mg (i.e. assuming a body weight of 70 kg). A patient receiving treatment may take a first dose upon waking and then a second dose in the evening (if on a two-dose regime) or at 3- or 4-hourly intervals thereafter. Alternatively, a slow release device may be used to provide optimal doses of the nucleic acid or vector according to the invention to a patient without the need to administer repeated doses.

However, the pharmaceutical vehicle may be a liquid, and the pharmaceutical composition is in the form of a solution. Liquid vehicles are used in preparing solutions, suspensions, emulsions, syrups, elixirs and pressurized compositions. The nucleic acid or vector according to the invention may be dissolved or suspended in a pharmaceutically acceptable liquid vehicle such as water, an organic solvent, a mixture of both or pharmaceutically acceptable oils or fats.

Liquid pharmaceutical compositions, which are sterile solutions or suspensions, can be utilized by, for example, intraocular, particularly intravitreal or subretinal injection. The nucleic acid or vector may be prepared as a sterile solid composition that may be dissolved or suspended at the time of administration using sterile water, saline, or other appropriate sterile injectable medium.

For instances in which retinal glial cells are to be contacted in vivo, an nucleic acid of the invention as described herein (e.g synthetic mRNA), or a vector, preferably an AAV vector, of the invention as described herein, or a recombinant AAV of the invention as described herein can be treated as appropriate for delivery to the eye. In particular, the present invention includes pharmaceutical compositions comprising an nucleic acid of the invention as described herein, a vector, preferably an AAV vector of the invention as described herein, or a recombinant AAV of the invention as described herein and a pharmaceutically-acceptable carrier, diluent or excipient. The nucleic acid of the invention as described herein, vector, preferably an AAV vector of the invention as described herein, or recombinant AAV of the invention as described herein can be combined with pharmaceutically-acceptable carriers, diluents and reagents useful in preparing a formulation that is generally safe, non-toxic, and desirable, and includes excipients that are acceptable for primate use. Such excipients can be solid, liquid, semisolid, or, in the case of an aerosol composition, gaseous. Examples of such carriers or diluents include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide.

Pharmaceutical compositions suitable for internal use in the present invention further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid to the extent that easy syringability exists. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the nucleic acid of the invention as described herein, vector, preferably an AAV vector, of the invention as described herein, or recombinant AAV of the invention as described herein in the required amount in an appropriate solvent with one or a combination of ingredients enumerated above, as required, followed by filtered sterilization. Generally, dispersions are prepared by incorporating the nucleic acid of the invention as described herein, vector, preferably AAV vector, of the invention as described herein, or recombinant AAV of the invention as described herein into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser, e.g. syringe, e.g. a prefilled syringe, together with instructions for administration.

The pharmaceutical compositions of the invention encompass any pharmaceutically acceptable salts, esters, or salts of such esters, or any other compound which, upon administration to an animal comprising a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bio-equivalents.

The term “pharmaceutically acceptable salt” refers to physiologically and pharmaceutically acceptable salts of the compounds of the invention: i.e., salts that retain the desired biological activity of the parent compound and do not impart undesired toxicological effects thereto. A variety of pharmaceutically acceptable salts are known in the art and described, e.g., in in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66:2 (1977). Also, for a review on suitable salts, see Handbook of Pharmaceutical Salts: Properties, Selection, and Use by Stahl and Wermuth (Wiley-VCH, 2002).

Pharmaceutically acceptable base addition salts are formed with metals or amines, such as alkali and alkaline earth metals or organic amines. Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

The nucleic acid of the invention as described herein, vector preferably AAV vector of the invention as described herein, or recombinant AAV of the invention as described herein, can be incorporated into pharmaceutical compositions for administration to mammalian patients, particularly primates and more particularly humans. The subject nucleic acid of the invention as described herein, vector, preferably AAV vector, of the invention as described herein, or recombinant AAV of the invention as described herein can be formulated in nontoxic, inert, pharmaceutically acceptable aqueous carriers, preferably at a pH ranging from 3 to 8, more preferably ranging from 6 to 8. Such sterile compositions will comprise the vector or virion containing the nucleic acid encoding the PHGDH or a biologically active fragment or variant thereof dissolved in an aqueous buffer having an acceptable pH upon reconstitution.

In some embodiments, the pharmaceutical composition provided herein comprise a therapeutically effective amount of a vector or virion in admixture with a pharmaceutically acceptable carrier and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer in which the pharmaceutical composition comprising the tumor suppressor gene contained in the adenoviral vector delivery system, may be in the range of 6.5 to 7.75, preferably 7 to 7.5, and most preferably 7.2 to 7.4.

### Promoters

Any promoter sequences that allow expression in glial cells (preferably retinal glial cells), which is the target tissue/cell type, are useful in the present invention. These include ubiquitous promoters e.g. CAG promoter, and glial cell-specific promoters. The CAG promoter preferably comprises the cytomegalovirus (CMV) early enhancer element, the promoter, the first exon and the first intron of chicken beta-actin (CBA) gene and the splice acceptor of the rabbit beta-globin gene. Examples of glial cell-specific promoters would include, but not be limited to, the promoters for GFAP, GLAST and RLBP1.

### Methods of Treatment

The present invention provides a method of treating a condition associated with or caused by degeneration, or loss, of photoreceptor cells in an individual in need thereof, the method comprising administering to the individual a cell or cell population generated in vitro or ex vivo by any method described herein.

The present invention provides a use of a cell or cell population generated in vitro or ex vivo by any method described herein in the manufacture of a medicament for the treatment of a condition associated with or caused by degeneration of photoreceptor cells in an individual in need thereof.

The present invention provides a cell or cell population generated in vitro or ex vivo by any method described herein for use in the treatment of a condition associated with or caused by degeneration of photoreceptor cells in an individual in need thereof.

In another aspect, the present invention provides a method of decreasing progression of or ameliorating vision loss associated with or cause by degeneration, or loss, of cone photoreceptor cells in a subject, the method comprising administering to the subject a nucleic acid of the invention as described herein, a vector, preferably an AAV vector, of the invention as described herein, a recombinant AAV of the invention as described herein, or a pharmaceutical composition of the invention as described herein, thereby of decreasing progression of or ameliorating vision loss associated with or cause by degeneration, or loss, of cone photoreceptor cells.

In another aspect, the present invention provides use of a nucleic acid of the invention as described herein, a vector, preferably an AAV vector, of the invention as described herein, a recombinant AAV of the invention as described herein, or a pharmaceutical composition of the invention as described herein, in the manufacture of a medicament for decreasing progression of or ameliorating vision loss associated with or cause by degeneration, or loss, of cone photoreceptor cells in a subject.

In another aspect, the present invention provides a nucleic acid of the invention as described herein, a vector, preferably an AAV vector, of the invention as described herein, a recombinant AAV of the invention as described herein, or a pharmaceutical composition of the invention as described herein, for use in decreasing progression of or ameliorating vision associated with or cause by degeneration, or loss, of cone photoreceptor cells in a subject.

In any aspect, preferably the subject is a human.

In any aspect or embodiment, the condition associated with or cause by degeneration, or loss, of cone photoreceptor cells may also be referred to as a cone cell disorder. The degeneration, or loss, of cone photoreceptor cells is associated with or causes changes in vision, typically a reduction in vision.

In some embodiments, the cone cell disorder is a retinal degenerative disorder. In certain embodiments, the retinal degenerative disorder is selected from the group consisting of achromatopsia, blue cone monochromacy, a protan defect, a deutan defect, and a tritan defect. In some embodiments, the cone cell disorder is a macular dystrophy or retinal dystrophy. The macular dystrophy may be selected from the group consisting of Stargardt's macular dystrophy, cone dystrophy (including rod-cone dystrophy and cone-rod dystrophy), Spinocerebellar ataxia type 7, and Bardet-Biedl syndrome-1. Preferably, the macular dystrophy is Stargardt's macular dystrophy or cone-rod dystrophy. In some embodiments, the cone cell disorder is a vision disorder of the central macula or a retinal dystrophy. In certain embodiments, vision disorder of the central macula or retinal dystrophy is selected from the group consisting of age-related macular degeneration, macular telangiectasia, retinitis pigmentosa, diabetic retinopathy, retinal vein occlusions, glaucoma, choroideremia, Sorsby's fundus dystrophy, adult vitelliform macular dystrophy, Best's disease, Leber's congenital amaurosis, and X-linked retinoschisis. Preferably, the vision disorder is retinitis pigmentosa, age-related macular degeneration or diabetic retinopathy.

In any embodiment, the subject has been diagnosed with a condition associated with or cause by degeneration, or loss, of cone photoreceptor cells as described herein. Preferably, the individual has been diagnosed with a cone dystrophy. The individual may have been diagnosed with progressive cone dystrophy or stationary cone dystrophy. The cone dystrophy may be a rod-cone dystrophy or a cone-rod dystrophy.

In some such embodiments, the method further comprises detecting a change in the condition or disorder symptoms. Including any symptom described herein. In some such embodiments, the change comprises a stabilization in the health of the existing or reprogrammed cone cells and/or a reduction in the rate of visual acuity loss of the subject. In certain such embodiments, the change comprises an improvement in in the visual acuity of the subject.

In some such embodiments, the method further comprises detecting a change in the condition or disorder symptoms, wherein the change comprises an increase in the ability of the subject to perceive a colour.

In any aspect of the present invention, the isolated nucleic acid of the invention as described herein, an AAV vector of the invention as described herein, a recombinant AAV of the invention as described herein, or a pharmaceutical composition of the invention as described herein, is administered to the retina of the subject, preferably by retinal injection (e.g. subretinal or intravitreal injection) into an affected eye of said subject.

In another aspect, the present invention provides for a composition comprising any of the AAV vectors or rAAV of the invention as disclosed herein and a pharmaceutically acceptable carrier, excipient or diluent.

In some embodiments, the loss of photoreceptors is a complete loss of cone photoreceptors. In some embodiments, the patient has eyesight of 20/60 or worse including 20/80 or worse, 20/100 or worse, 20/120 or worse, 20/140 or worse, 20/160 or worse, 20/180 or worse, 20/200 or worse, 20/400 or worse, 20/800 or worse, or 20/1000 or worse.

Administration of a cell or cell population to an individual in need thereof to treat a condition associated with or cause by degeneration of cone photoreceptor cells, may be by any method known in the art.

In the methods of the invention, cells to be transplanted are transferred to a recipient in any physiologically acceptable excipient comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996. Choice of the cellular excipient and any accompanying elements of the composition will be adapted in accordance with the route and device used for administration. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium.

The pharmaceutical preparations of the invention are optionally packaged in a suitable container with written instructions for a desired purpose. Such formulations may comprise a cocktail of retinal differentiation and/or trophic factors, in a form suitable for combining with cell or cell population of the invention as described herein. Such a composition may further comprise suitable buffers and/or excipients appropriate for transfer into an animal.

The cell or cell population of the invention as described herein may be formulated with a pharmaceutically acceptable carrier. For example, cell or cell population of the invention as described herein may be administered alone or as a component of a pharmaceutical formulation. The subject compounds may be formulated for administration in any convenient way for use in medicine. Pharmaceutical preparations suitable for administration may comprise the cell or cell population of the invention as described herein in combination with one or more pharmaceutically acceptable sterile isotonic aqueous or nonaqueous solutions (e.g., balanced salt solution (BSS)), dispersions, suspensions or emulsions, or sterile powders which may be reconstituted into sterile injectable solutions or dispersions just prior to use, which may contain antioxidants, buffers, bacteriostats, solutes or suspending or thickening agents. Exemplary pharmaceutical preparations comprises the cell or cell population of the invention as described herein in combination with ALCON® BSS PLUS® (a balanced salt solution containing, in each mL, sodium chloride 7.14 mg, potassium chloride 0.38 mg, calcium chloride dihydrate 0.154 mg, magnesium chloride hexahydrate 0.2 mg, dibasic sodium phosphate 0.42 mg, sodium bicarbonate 2.1 mg, dextrose 0.92 mg, glutathione disulfide (oxidized glutathione) 0.184 mg, hydrochloric acid and/or sodium hydroxide (to adjust pH to approximately 7.4) in water).

When administered, the pharmaceutical preparations for use in this disclosure may be in a pyrogen-free, physiologically acceptable form.

The preparation comprising a cell or cell population of the invention as described herein used in the methods described herein may be transplanted in a suspension, gel, colloid, slurry, or mixture. Further, the preparation may desirably be encapsulated or injected in a viscous form into the vitreous humor for delivery to the site of retinal or choroidal damage. Also, at the time of injection, cryopreserved cell or cell population of the invention as described herein may be resuspended with commercially available balanced salt solution to achieve the desired osmolality and concentration for administration by subretinal injection. The preparation may be administered to an area of the pericentral macula that was not completely lost to disease, which may promote attachment and/or survival of the administered cells.

The cell or cell population of the invention as described herein may be frozen (cryopreserved) as described herein. Upon thawing, the viability of such cells may be at least 20%, at least 30%, at least 40%, at least 50%, at least 60%, at least 70%, at least 80%, at least 90% at least 95% or about 100% (e.g., at least 20%, at least 30%, at least 40%, at least 50%, at least 60%, at least 70%, at least 80%, at least 90% at least 95% or about 100% of the cells harvested after thawing are viable or at least 20%, at least 30%, at least 40%, at least 50%, at least 60%, at least 70%, at least 80%, at least 90% at least 95% or about 100% of the cell number initially frozen are harvested in a viable state after thawing). In some instances, the viability of the cells prior to and after thawing is about 80%. In some instances, at least 90% or at least 95% or about 95% of cells that are frozen are recovered. The cells may be frozen as single cells or as aggregates.

The cell or cell population of the invention as described herein may be delivered in a pharmaceutically acceptable ophthalmic formulation by intraocular injection. When administering the formulation by intravitreal injection, for example, the solution may be concentrated so that minimized volumes may be delivered. Concentrations for injections may be at any amount that is effective and non-toxic, depending upon the factors described herein. The pharmaceutical preparations of cell or cell population of the invention as described herein for treatment of a patient may be formulated at doses of at least about 10⁴ cells/mL. The cell or cell population of the invention as described herein preparations for treatment of a patient are formulated at doses of at least about 10³, 10⁴, 10⁵, 10⁶, 10⁷, 10⁸, 10⁹, or 10¹⁰ cells/mL.

The pharmaceutical preparations of cells of the invention described herein may comprise at least about 1,000; 2,000; 3,000; 4,000; 5,000; 6,000; 7,000; 8,000; or 9,000 cone or cone-like photoreceptor cells. The pharmaceutical preparations of cone or cone-like photoreceptor cells may comprise at least about 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ cone photoreceptor cells. The pharmaceutical preparations of cone or cone-like photoreceptor cells may comprise at least about 1×10²-1×10³, 1×10²-1×10⁴, 1×10⁴, 1×10⁵, or 1×10³-1×10⁶ cone or cone-like photoreceptor cells. The pharmaceutical preparations of cone or cone-like photoreceptor cells may comprise at least about 10,000, 20,000, 25,000, 50,000, 75,000, 100,000, 125,000, 150,000, 175,000, 180,000, 185,000, 190,000, or 200,000 cone or cone-like photoreceptor cells. For example, the pharmaceutical preparation of cone or cone-like photoreceptor cells may comprise at least about 20,000-200,000 cone or cone-like photoreceptor cells in a volume at least about 50-200 μL. Further, the pharmaceutical preparation of cone or cone-like photoreceptor cells may comprise about 50,000 photoreceptor is in a volume of 150 μL, about 200,000 cone or cone-like photoreceptor cells in a volume of 150 μL, or at least about 180,000 photoreceptor cells in a volume at least about 150 μL.

In the aforesaid pharmaceutical preparations and compositions, the number of cone or cone-like photoreceptor cells or concentration of photoreceptor cells may be determined by counting viable cells and excluding non-viable cells. For example, non-viable photoreceptor may be detected by failure to exclude a vital dye (such as Trypan Blue), or using a functional assay (such as the ability to adhere to a culture substrate, phagocytosis, etc.). Additionally, the number of photoreceptor cells or concentration of photoreceptor cells may be determined by counting cells that express one or more photoreceptor cell markers and/or excluding cells that express one or more markers indicative of a cell type other than photoreceptor.

The cone or cone-like photoreceptor cells may be formulated for delivery in a pharmaceutically acceptable ophthalmic vehicle, such that the preparation is maintained in contact with the ocular surface for a sufficient time period to allow the cells to penetrate the affected regions of the eye, as for example, the anterior chamber, posterior chamber, vitreous body, aqueous humor, vitreous humor, cornea, iris/ciliary, lens, choroid, retina (e.g. sub-retina), sclera, suprachoroidal space, conjunctiva, subconjunctival space, episcleral space, intracorneal space, epicorneal space, pars plana, surgically-induced avascular regions, or the macula.

The methods described herein may further comprise the step of monitoring the efficacy of treatment or prevention by measuring electroretinogram responses, optomotor acuity threshold, or luminance threshold in the subject. The method may also comprise monitoring the efficacy of treatment or prevention by monitoring immunogenicity of the cells or migration of the cells in the eye.

Note, that the human cells may be used in human patients, as well as in animal models or animal patients. For example, the human cells may be tested in mouse, rat, cat, dog, or non-human primate models of retinal degeneration. Additionally, the human cells may be used therapeutically to treat animals in need thereof, such as in veterinary medicine. Examples of veterinary subjects or patients include without limitation dogs, cats, and other companion animals, and economically valuable animals such as livestock and horses.

In addition to the use of cone or cone-like photoreceptor cells that have been generated in vitro or in vivo as described above, also contemplated is the use of gene therapy approach to reprogram cells to cone or cone-like photoreceptor cells in situ or in vivo.

As alluded to above, the subject nucleic acids and gene delivery vectors as described herein, referred to collectively herein as the “subject compositions”, find use in expressing a transgene in cone cells of an animal. For example, the subject compositions may be used in research, e.g. to determine the effect that the gene has on cone cell viability and/or function. As another example, the subject compositions may be used in medicine, e.g. to treat a cone cell disorder. Thus, in some aspects of the invention, methods are provided for the expression of a gene in cone cells, the method comprising contacting cone cells with a composition of the present disclosure. In some embodiments, contacting occurs in vitro. In some embodiments, contacting occurs in vivo, i.e., the subject composition is administered to a subject.

For instances in which cells are to be contacted in vitro with a subject nucleic acid or gene delivery vector as described herein, the cells may be from any mammalian species, e.g. rodent (e.g. mice, rats, gerbils, squirrels), rabbit, feline, canine, goat, ovine, pig, equine, bovine, primate, human.

For instances in which cells are to be contacted in vivo with a subject nucleic acid or gene delivery vector as described herein, the subject may be any mammal, e.g. rodent (e.g. mice, rats, gerbils), rabbit, feline, canine, goat, ovine, pig, equine, bovine, or primate.

The methods and compositions of the present disclosure find use in the treatment of any condition that can be addressed, at least in part, by producing functional cone photoreceptor cells. Thus, the compositions and methods of the present disclosure find use in the treatment of individuals in need of a cone cell therapy. By a person in need of a cone cell therapy, it is meant an individual having or at risk of developing a cone cell disorder. By a “cone cell disorder” it is meant any disorder impacting retinal cone cells, including but not limited to vision disorders of the eye that are associated with a defect within cone cells, i.e. a cone-intrinsic defect, e.g. macular dystrophies such as Stargardt's macular dystrophy, cone dystrophy, cone-rod dystrophy, Spinocerebellar ataxia type 7, and Bardet-Biedl syndrome-1; as well as color vision disorders, including achromatopsia, incomplete achromatopsia, blue cone monochromacy, and protan, deutan, and tritan defects; as well as vision disorders of the central macula (within primates) that may be treated by targeting cone cells, e.g. age-related macular degeneration, macular telangiectasia, retinitis pigmentosa, diabetic retinopathy, retinal vein occlusions, glaucoma, Sorsby's fundus dystrophy, adult vitelliform macular dystrophy, Best's disease, rod-cone dystrophy, Leber's congenital amaurosis, and X-linked retinoschisis.

Stargardt's macular dystrophy. Stargardt's macular dystrophy, also known as Stargardt Disease and fundus flavimaculatus, is an inherited form of juvenile macular degeneration that causes progressive vision loss usually to the point of legal blindness. The onset of symptoms usually appears between the ages of six and thirty years old (average of about 16-18 years). Mutations in several genes, including ABCA4, CNGB3, ELOVL4, PROM1, are associated with the disorder. Symptoms typically develop by twenty years of age, and include wavy vision, blind spots, blurriness, impaired color vision, and difficulty adapting to dim lighting. The main symptom of Stargardt disease is loss of visual acuity, which ranges from 20/50 to 20/200. In addition, those with Stargardt disease are sensitive to glare; overcast days offer some relief. Vision is most noticeably impaired when the macula is damaged, which can be observed by fundus exam.

Cone dystrophy. Cone dystrophy (COD) is an inherited ocular disorder characterized by the loss of cone cells. The most common symptoms of cone dystrophy are vision loss (age of onset ranging from the late teens to the sixties), sensitivity to bright lights, and poor color vision. Visual acuity usually deteriorates gradually, but it can deteriorate rapidly to 20/200; later, in more severe cases, it drops to “counting fingers” vision. Color vision testing using color test plates (HRR series) reveals many errors on both red-green and blue-yellow plates. It is believed that the dystrophy is primary, since subjective and objective abnormalities of cone function are found before ophthalmoscopic changes can be seen. However, the retinal pigment epithelium (RPE) rapidly becomes involved, leading to a retinal dystrophy primarily involving the macula. The fundus exam via ophthalmoscope is essentially normal early on in cone dystrophy, and definite macular changes usually occur well after visual loss. The most common type of macular lesion seen during ophthalmoscopic examination has a bull's-eye appearance and consists of a doughnut-like zone of atrophic pigment epithelium surrounding a central darker area. In another, less frequent form of cone dystrophy there is rather diffuse atrophy of the posterior pole with spotty pigment clumping in the macular area. Rarely, atrophy of the choriocapillaris and larger choroidal vessels is seen in patients at an early stage. Fluorescein angiography (FA) is a useful adjunct in the workup of someone suspected to have cone dystrophy, as it may detect early changes in the retina that are too subtle to be seen by ophthalmoscope. Because of the wide spectrum of fundus changes and the difficulty in making the diagnosis in the early stages, electroretinography (ERG) remains the best test for making the diagnosis. Abnormal cone function on the ERG is indicated by a reduced single-flash and flicker response when the test is carried out in a well-lit room (photopic ERG). Mutations in several genes, including GUCA1A, PDE6C, PDE6H, and RPGR, are associated with the disorder.

Spinocerebellar ataxia type 7. Spinocerebellar ataxia is a progressive, degenerative, inherited disease characterized by slowly progressive incoordination of gait and is often associated with poor coordination of hands, speech, and eye movements. There are multiple types of SCA, with Spinocerebellar ataxia type 7 (SCA-7) differing from most other SCAs in that visual problems can occur in addition to poor coordination. SCA-7 is associated with autosomal dominant mutations in the ATXN7/SCA7 gene. When the disease manifests itself before age 40, visual problems rather than poor coordination are typically the earliest signs of disease. Early symptoms include difficulty distinguishing colors and decreased central vison. In addition, symptoms of ataxia (incoordination, slow eye movements, and mild changes in sensation or reflexes) may be detectable. Loss of motor control, unclear speech, and difficulty swallowing become prominent as the disease progresses.

Bardet-Biedl syndrome-1. Bardet-Biedl syndrome-1 (BBS-1) is a pleiotropic disorder with variable expressivity and a wide range of clinical variability observed both within and between families. The main clinical features are rod-cone dystrophy, with childhood-onset visual loss preceded by night blindness; postaxial polydactyly; truncal obesity that manifests during infancy and remains problematic throughout adulthood; specific learning difficulties in some but not all individuals; male hypogenitalism and complex female genitourinary malformations; and renal dysfunction, a major cause of morbidity and mortality. Vision loss is one of the major features of Bardet-Biedl syndrome. Problems with night vision become apparent by mid-childhood, followed by blind spots that develop in the peripheral vision. Over time, these blind spots enlarge and merge to produce tunnel vision. Most people with Bardet-Biedl syndrome also develop blurred central vision (poor visual acuity) and become legally blind by adolescence or early adulthood. Bardet-Biedl syndrome can result from mutations in at least 14 different genes (often called BBS genes) known or suspected to play critical roles in cilia function, with mutations in BBS1 and BBS10 being the most common.

Achromatopsia. Achromatopsia, or Rod monochromatism, is a disorder in which subjects experience a complete lack of the perception of color, such that the subject sees only in black, white, and shades of grey. Other symptoms include reduced visual acuity, photophobia, nystagmus, small central scotoma, and eccentric fixation. The disorder is frequently noticed first in children around six months of age by their photophobic activity and/or their nystagmus. Visual acuity and stability of the eye motions generally improve during the first 6-7 years of life (but remain near 20/200). Mutations in CNGB3, CNGA3, GNAT2, PDE6C, and PDE6HI have been associated with the disorder.

Incomplete achromatopsia. Incomplete achromatopsia is similar to Achromatopsia but with less penetrance. In incomplete achromatopsia, the symptoms are similar to those of complete achromatopsia except in a diminished form. Individuals with incomplete achromatopsia have reduced visual acuity with or without nystagmus or photophobia. Furthermore, these individuals show only partial impairment of cone cell function but again have retained rod cell function.

Blue cone monochromacy. Blue cone (S cone) monochromatism (BCM) is a rare X-linked congenital stationary cone dysfunction syndrome, affecting approximately 1 in 100,000 individuals. Affected males with BCM have no functional long wavelength sensitive (L) or medium wavelength sensitive (M) cones in the retina, due to mutations at the genetic locus for the L and M-opsin genes. Color discrimination is severely impaired from birth, and vision is derived from the remaining preserved S cones and rod photoreceptors. BCM typically presents with reduced visual acuity (6/24 to 6/60), pendular nystagmus, photophobia, and patients often have myopia. The rod-specific and maximal electroretinogram (ERG) usually show no definite abnormality, whereas the 30 Hz cone ERG cannot be detected. Single flash photopic ERG is often recordable, albeit small and late, and the S cone ERG is well preserved.

Color vision deficiency. Color vision deficiency (CVD), or color blindness, is the inability or decreased ability to see color, or perceive color differences, under normal lighting conditions. Individuals suffering from color blindness may be identified as such using any of a number of color vision tests, e.g., color ERG (cERG), pseudoisochromatic plates (Ishihara plates, Hardy-Rand-Ritter polychromatic plates), the Farnsworth-Munsell 100 hue test, the Farnsworth's panel D-15, the City University test, Kollner's rule, etc. Examples of color vision deficiencies include protan defects, deutan defects, and tritan defects. Protan defects include protanopia (an insensitivity to red light) and protanomaly (a reduced sensitivity to red light), and are associated with mutations in the L-Opsin gene (OPN1LW). Deutan defects include deuteranopia (an insensitivity to green light) and deutanomaly (a reduced sensitivity to green light), and are associated with mutations in the M-Opsin gene (OPN1MW). Tritan defects include tritanopia (an insensitivity to blue light) and tritanomaly (a reduced sensitivity to blue light), and are associated with mutations in the S-Opsin gene (OPN1SW).

Age-related macular degeneration. Age-related macular degeneration (AMD) is one of the leading causes of vision loss in people over the age of 50 years. AMD mainly affects central vision, which is needed for detailed tasks such as reading, driving, and recognizing faces. The vision loss in this condition results from a gradual deterioration of photoreceptors in the macula. Side (peripheral) vision and night vision are generally not affected.

Researchers have described two major types of age-related macular degeneration, known as the dry, or “nonexudative” form, and the wet, or “exudative” or “neovascular”, form, both of which may be treated by delivering transgenes in the context of the subject polynucleotide cassettes.

Dry AMD is characterized by a buildup of yellow deposits called drusen between the retinal pigment epithelium and the underlying choroid of the macula, which may be observed by Fundus photography. This results in a slowly progressive loss of vision. The condition typically affects vision in both eyes, although vision loss often occurs in one eye before the other. Other changes may include pigment changes and RPE atrophy. For example, in certain cases called central geographic atrophy, or “GA”, atrophy of the retinal pigment epithelial and subsequent loss of photoreceptors in the central part of the eye is observed. Dry AMD has been associated with mutations in CD59 and genes in the complement cascade.

Wet AMD is a progressed state of dry AMD, and occurs in about 10% of dry AMD patients. Pathological changes include retinal pigment epithelial cells (RPE) dysfunction, fluid collecting under the RPE, and choroidal neovascularization (CNV) in the macular area. Fluid leakage, RPE or neural retinal detachment and bleeding from ruptured blood vessels can occur in severe cases. Symptoms of wet AMD may include visual distortions, such as straight lines appearing wavy or crooked, a doorway or street sign looking lopsided, or objects appearing smaller or farther away than they really are; decreased central vision; decreased intensity or brightness of colors; and well-defined blurry spot or blind spot in the field of vision. Onset may be abrupt and worsen rapidly. Diagnosis may include the use of an Amsler grid to test for defects in the subject's central vision (macular degeneration may cause the straight lines in the grid to appear faded, broken or distorted), fluorescein angiogram to observe blood vessel or retinal abnormalities, and optical coherence tomography to detect retina swelling or leaking blood vessels. A number of cellular factors have been implicated in the generation of CNV, among which are vascular endothelial growth factor (VEGF), platelet-derived growth factor (PDGF), pigment epithelium-derived factor (PEDF), hypoxia inducible factor (HIF), angiopoietin (Ang), and other cytokines, mitogen-activated protein kinases (MAPK) and others.

Macular telangiectasia. Macular telangiectasia (MacTel) is a form of pathologically dilated blood vessels (telangiectasia) in the parafoveal region of the macula. The tissue deteriorates and the retinal structure becomes scarred due to the development of liquid-filled cysts, which impairs nutrition of the photoreceptor cells and destroys vision permanently. There are two types of MacTel, type 1 and type 2. Macular telangiectasia type 2 is a bilateral disease, whose prevalence has recently been shown to be as high as 0.1% in persons 40 years and older. Biomicroscopy may show reduced retinal transparency, crystalline deposits, mildly ectatic capillaries, blunted venules, retinal pigment plaques, foveal atrophy, and neovascular complexes. Fluorescein angiography shows telangiectatic capillaries predominantly temporal to the foveola in the early phase and a diffuse hyperfluorescence in the late phase. High-resolution optical coherence tomography (OCT) may reveal disruption of the photoreceptor inner segment-outer segment border, hyporeflective cavities at the level of the inner or outer retina, and atrophy of the retina in later stages. In Type 1 macular telangiectasia, the disease almost always occurs in one eye, which differentiates it from Type 2. While MacTel does not usually cause total blindness, it commonly causes loss of the central vision, which is required for reading and driving vision, over a period of 10-20 years.

Retinitis pigmentosa. Retinitis Pigmentosa (RP) is a group of inherited disorders characterized by progressive peripheral vision loss and night vision difficulties (nyctalopia) that can lead to central vision loss. Presenting signs and symptoms of RP vary, but the classic ones include nyctalopia (night blindness, most commonly the earliest symptom in RP); visual loss (usually peripheral, but in advanced cases, central visual loss); and photopsia (seeing flashes of light). Because RP is a collection of many inherited diseases, significant variability exists in the physical findings. Ocular examination involves assessment of visual acuity and pupillary reaction, as well as anterior segment, retinal, and funduscopic evaluation. In some instances, the RP is one aspect of a syndrome, e.g. syndromes that are also associated with hearing loss (Usher syndrome, Waardenburg syndrome, Alport syndrome, Refsum disease); Kearns-Sayre syndrome (external ophthalmoplegia, lid ptosis, heart block, and pigmentary retinopathy); Abetalipoproteinemia (Fat malabsorption, fat-soluble vitamin deficiencies, spinocerebellar degeneration, and pigmentary retinal degeneration); mucopolysaccharidoses (eg, Hurler syndrome, Scheie syndrome, Sanfilippo syndrome); Bardet-Biedl syndrome (Polydactyly, truncal obesity, kidney dysfunction, short stature, and pigmentary retinopathy); and neuronal ceroid lipofuscinosis (Dementia, seizures, and pigmentary retinopathy; infantile form is known as Jansky-Bielschowsky disease, juvenile form is Vogt-Spielmeyer-Batten disease, and adult form is Kufs syndrome). Retinitis pigmentosa is most commonly associated with mutations in the RHO, RP2, RPGR, RPGRIP1, PDE6A, PDE6B, MERTK, PRPH2, CNGB1, USH2A, ABCA4, BBS genes.

Diabetic retinopathy. Diabetic retinopathy (DR) is damage to the retina caused by complications of diabetes, which can eventually lead to blindness. Without wishing to be bound by theory, it is believed that hyperglycemia-induced intramural pericyte death and thickening of the basement membrane lead to incompetence of the vascular walls. These damages change the formation of the blood-retinal barrier and also make the retinal blood vessels become more permeable.

There are two stages of diabetic retinopathy: non-proliferative diabetic retinopathy (NPDR), and proliferative diabetic retinopathy (PDR). Nonproliferative diabetic retinopathy is the first stage of diabetic retinopathy, and is diagnosed by fundoscopic exam and coexistent diabetes. In cases of reduced vision, fluorescein angiography may be done to visualize the vessels in the back of the eye to and any retinal ischemia that may be present. All people with diabetes are at risk for developing NPDR, and as such, would be candidates for prophylactic treatment with the subject vectors. Proliferative diabetic retinopathy is the second stage of diabetic retinopathy, characterized by neovascularization of the retina, vitreous hemorrhage, and blurred vision. In some instances, fibrovascular proliferation causes tractional retinal detachment. In some instances, the vessels can also grow into the angle of the anterior chamber of the eye and cause neovascular glaucoma. Individuals with NPDR are at increased risk for developing PDR, and as such, would be candidates for prophylactic treatment with the subject vectors.

Diabetic macular edema. Diabetic macular edema (DME) is an advanced, vision-limiting complication of diabetic retinopathy that affects nearly 30% of patients who have had diabetes for at least 20 years, and is responsible for much of the vision loss due to DR. It results from retinal microvascular changes that compromise the blood-retinal barrier, causing leakage of plasma constituents into the surrounding retina and, consequently, retinal edema. Without wishing to be bound by theory, it is believed that hyperglycemia, sustained alterations in cell signaling pathways, and chronic microvascular inflammation with leukocyte-mediated injury leads to chronic retinal microvascular damage, which triggers an increase in intraocular levels of VEGF, which in turn increases the permeability of the vasculature.

Patients at risk for developing DME include those who have had diabetes for an extended amount of time and who experience one or more of severe hypertension (high blood pressure), fluid retention, hypoalbuminemia, or hyperlipidemia. Common symptoms of DME are blurry vision, floaters, double vision, and eventually blindness if the condition is allowed to progress untreated. DME is diagnosed by funduscopic examination as retinal thickening within 2 disc diameters of the center of the macula. Other methods that may be employed include Optical coherence tomography (OCT) to detect retinal swelling, cystoid edema, and serous retinal detachment; fluorescein angiography, which distinguishes and localizes areas of focal versus diffuse leakage, thereby guiding the placement of laser photocoagulation if laser photocoagulation is to be used to treat the edema; and color stereo fundus photographs, which can be used to evaluate long-term changes in the retina. Visual acuity may also be measured, especially to follow the progression of macular edema and observe its treatment following administration of the subject pharmaceutical compositions.

Retinal vein occlusions. A retinal vein occlusion (RVO) is a blockage of the portion of the circulation that drains the retina of blood. The blockage can cause back-up pressure in the capillaries, which can lead to hemorrhages and also to leakage of fluid and other constituents of blood.

Glaucoma. Glaucoma is a term describing a group of ocular (eye) disorders that result in optic nerve damage, often associated with increased fluid pressure in the eye (intraocular pressure) (IOP). The disorders can be roughly divided into two main categories, “open-angle” and “closed-angle” (or “angle closure”) glaucoma. Open-angle glaucoma accounts for 90% of glaucoma cases in the United States. It is painless and does not have acute attacks. The only signs are gradually progressive visual field loss, and optic nerve changes (increased cup-to-disc ratio on fundoscopic examination). Closed-angle glaucoma accounts for less than 10% of glaucoma cases in the United States, but as many as half of glaucoma cases in other nations (particularly Asian countries). About 10% of patients with closed angles present with acute angle closure crises characterized by sudden ocular pain, seeing halos around lights, red eye, very high intraocular pressure (>30 mmHg), nausea and vomiting, suddenly decreased vision, and a fixed, mid-dilated pupil. It is also associated with an oval pupil in some cases. Modulating the activity of proteins encoded by DLK, NMDA, INOS, CASP-3, Bcl-2, or Bcl-xl may treat the condition.

Sorsby's fundus dystrophy. Sorsby's fundus dystrophy is an autosomal dominant, retinal disease associated with mutations in the TIMP3 gene. Clinically, early, mid-peripheral, drusen and colour vision deficits are found. Some patients complain of night blindness. Most commonly, the presenting symptom is sudden acuity loss, manifest in the third to fourth decades of life, due to untreatable submacular neovascularisation. Histologically, there is accumulation of a confluent lipid containing material 30 μm thick at the level of Bruch's membrane.

Vitelliform macular dystrophy. Vitelliform macular dystrophy is a genetic eye disorder that can cause progressive vision loss. Vitelliform macular dystrophy is associated with the buildup of fatty yellow pigment (lipofuscin) in cells underlying the macula. Over time, the abnormal accumulation of this substance can damage cells that are critical for clear central vision. As a result, people with this disorder often lose their central vision, and their eyesight may become blurry or distorted. Vitelliform macular dystrophy typically does not affect side (peripheral) vision or the ability to see at night.

Researchers have described two forms of vitelliform macular dystrophy with similar features. The early-onset form (known as Best disease) usually appears in childhood; the onset of symptoms and the severity of vision loss vary widely. It is associated with mutations in the VMD2/BEST1 gene. The adult-onset form (Adult vitelliform macular dystrophy) begins later, usually in mid-adulthood, and tends to cause vision loss that worsens slowly over time. It has been associated with mutations in the PRPH2 gene. The two forms of vitelliform macular dystrophy each have characteristic changes in the macula that can be detected during an eye examination.

Leber's congenital amaurosis. Leber's congenital amaurosis (LCA) is a severe dystrophy of the retina that typically becomes evident in the first year of life. Visual function is usually poor and often accompanied by nystagmus, sluggish or near-absent pupillary responses, photophobia, high hyperopia, and keratoconus. Visual acuity is rarely better than 20/400. A characteristic finding is Franceschetti's oculo-digital sign, comprising eye poking, pressing, and rubbing. The appearance of the fundus is extremely variable. While the retina may initially appear normal, a pigmentary retinopathy reminiscent of retinitis pigmentosa is frequently observed later in childhood. The electroretinogram (ERG) is characteristically “nondetectable” or severely subnormal. Mutations in 17 genes are known to cause LCA: GUCY2D (locus name: LCA1), RPE65 (LCA2), SPATA7 (LCA3), AIPL1 (LCA4), LCAS (LCAS), RPGRIP1 (LCA6), CRX (LCAT), CRB1 (LCA8), NMNAT1 (LCA9), CEP290 (LCA10), IMPDH1 (LCA11), RD3 (LCA12), RDH12 (LCA13), LRAT (LCA14), TULP1 (LCA15), KCNJ13 (LCA16), and IQCB1. Together, mutations in these genes are estimated to account for over half of all LCA diagnoses. At least one other disease locus for LCA has been reported, but the gene is not known.

X-linked retinoschisis. X-linked retinoschisis (XLRS) is characterized by symmetric bilateral macular involvement with onset in the first decade of life, in some cases as early as age three months. Fundus examination shows areas of schisis (splitting of the nerve fiber layer of the retina) in the macula, sometimes giving the impression of a spoke wheel pattern. Schisis of the peripheral retina, predominantly inferotemporally, occurs in approximately 50% of individuals. Affected males typically have vision of 20/60 to 20/120. Visual acuity often deteriorates during the first and second decades of life but then remains relatively stable until the fifth or sixth decade. The diagnosis of X-linked juvenile retinoschisis is based on fundus findings, results of electrophysiologic testing, and molecular genetic testing. RS1 is the only gene known to be associated with X-linked juvenile retinoschisis.

An individual affected by a cone cell disorder or at risk for developing a cone cell disorder can be readily identified using techniques to detect the symptoms of the disorder as known in the art, including, without limitation, fundus photography; Optical coherence tomography (OCT); adaptive optics (AO); electroretinography, e.g. ERG, color ERG (cERG); color vision tests such as pseudoisochromatic plates (Ishihara plates, Hardy-Rand-Ritter polychromatic plates), the Farnsworth-Munsell 100 hue test, the Farnsworth's panel D-15, the City university test, Kollner's rule, and the like; and visual acuity tests such as the ETDRS letters test, Snellen visual acuity test, visual field test, contrast sensitivity test, and the like; as will be known by the ordinarily skilled artisan. Additionally or alternatively, the individual affected by a cone cell disorder or at risk for developing a cone cell disorder can be readily identified using techniques to detect gene mutations that are associated with the cone cell disorder as known in the art, including, without limitation, PCR, DNA sequence analysis, restriction digestion, Southern blot hybridization, mass spectrometry, etc. In some embodiments, the method comprises the step of identifying the individual in need of a cone cell therapy. In such instances, any convenient method for determining if the individual has the symptom(s) of a cone cell disorder or is at risk for developing a cone cell disorder, for example by detecting the symptoms described herein or known in the art, by detecting a mutation in a gene as herein or as known in the art, etc. may be utilized to identify the individual in need of a cone cell therapy.

### Administration

In practicing the in vivo methods, a composition for in vivo reprogramming is typically delivered to the retina of the subject in an amount that is effective to result in the expression of, for example, the transgene(s) in the retinal glial cells. In some embodiments, the method comprises the step of detecting the expression of the transgene in cells of the retina, for example retinal glial cells.

In a preferred embodiment, nucleic acids, vectors, AAVs, medicaments according to the invention may be administered to a subject by injection into the blood stream, a nerve or directly into a site requiring treatment, i.e. the eye. For example, the medicament may be injected at least adjacent the retina. Injections may be intravenous (bolus or infusion) or subcutaneous (bolus or infusion), or intradermal (bolus or infusion), or intravitreal (bolus or infusion), or subretinal (bolus or infusion).

Preferably, the nucleic acid of the invention as described herein, vector, preferably AAV vector of the invention as described herein, recombinant AAV of the invention as described herein, or in vitro or ex vivo reprogrammed cells (or composition containing the cells thereof) is administered directly to the subject's eye, preferably to the retina, preferably by injection, more preferably retinal injection (e.g subretinal injection), or most preferably by intravitreal injection.

The composition may be administered to the retina of the by any suitable method. For example, the composition may be administered intraocularly via intravitreal injection or subretinal injection. The general methods for delivering a nucleic acid or vector via intravitreal injection or via subretinal injection may be illustrated by the following brief outlines. These examples are merely meant to illustrate certain features of the methods, and are in no way meant to be limiting.

For subretinal administration, the nucleic acid or vector can be delivered in the form of a suspension injected subretinally under direct observation using an operating microscope. Typically, a volume of 1 to 200 uL, e.g. 50 uL, 100 uL, 150 ul, or 200 uL, but usually no more than 200 uL, of the subject composition will be administered by such methods. This procedure may involve vitrectomy followed by injection of vector suspension using a fine cannula through one or more small retinotomies into the subretinal space. Briefly, an infusion cannula can be sutured in place to maintain a normal globe volume by infusion (of e.g. saline) throughout the operation. A vitrectomy is performed using a cannula of appropriate bore size (for example 20 to 27 gauge), wherein the volume of vitreous gel that is removed is replaced by infusion of saline or other isotonic solution from the infusion cannula. The vitrectomy is advantageously performed because (1) the removal of its cortex (the posterior hyaloid membrane) facilitates penetration of the retina by the cannula; (2) its removal and replacement with fluid (e.g. saline) creates space to accommodate the intraocular injection of the nucleic acid or vector, and (3) its controlled removal reduces the possibility of retinal tears and unplanned retinal detachment.

For intravitreal administration, the nucleic acid or vector can be delivered in the form of a suspension. Initially, topical anesthetic is applied to the surface of the eye followed by a topical antiseptic solution. The eye is held open, with or without instrumentation, and the nucleic acid or vector is injected through the sclera with a short, narrow, for example a 30 gauge needle, into the vitreous cavity of the eye of a subject under direct observation. Typically, a volume of 1 to 100 uL, e.g. 25 uL, 50 uL, or 100 uL, and usually no more than 100 uL, of the subject composition may be delivered to the eye by intravitreal injection without removing the vitreous. Alternatively, a vitrectomy may be performed, and the entire volume of vitreous gel is replaced by an infusion of the subject composition. In such cases, up to about 4 mL of the subject composition may be delivered, e.g. to a human eye. Intravitreal administration is generally well tolerated. At the conclusion of the procedure, there is sometimes mild redness at the injection site. There is occasional tenderness, but most patients do not report any pain. No eye patch or eye shield is necessary after this procedure, and activities are not restricted. Sometimes, an antibiotic eye drop is prescribed for several days to help prevent infection.

In practicing the methods, the composition is typically delivered to the retina of the subject in an amount that is effective to result in the expression of the transgene(s) in the cone cells. In some embodiments, the method comprises the step of detecting the expression of the transgene(s) in the cells of the retina.

There are a number of ways to detect the expression of a transgene, any of which may be used in the subject embodiments. For example, expression may be detected directly, i.e. by measuring the amount of gene product, for example, at the RNA level, e.g. by RT-PCR, Northern blot, RNAse protection; or at the protein level, e.g. by Western blot, ELISA, immunohistochemistry, and the like. As another example, expression may be detected indirectly, i.e. by detecting the impact of the gene product on the viability or function of the cone photoreceptor in the subject. For example, if the gene product encoded by the transgene improves the viability of the cone cell, the expression of the transgene may be detected by detecting an improvement in viability of the cone cell, e.g. by fundus photography, Optical coherence tomography (OCT), Adaptive Optics (AO), and the like. If the gene product encoded by the transgene alters the activity of the cone cell, the expression of the transgene may be detected by detecting a change in the activity of the cone cell, e.g. by electroretinogram (ERG) and color ERG (cERG); functional adaptive optics; color vision tests such as pseudoisochromatic plates (Ishihara plates, Hardy-Rand-Ritter polychromatic plates), the Farnsworth-Munsell 100 hue test, the Farnsworth's panel D-15, the City university test, Kollner's rule, and the like; and visual acuity tests such as the ETDRS letters test, Snellen visual acuity test, visual field test, contrast sensitivity test, and the like, as a way of detecting the presence of the delivered polynucleotide. In some instances, both an improvement in viability and a modification in cone cell function may be detected.

In some embodiments, the method results in a therapeutic benefit, e.g. preventing the development of a disorder, halting the progression of a disorder, reversing the progression of a disorder, etc. In some embodiments, the method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy. For example, therapeutic efficacy in treating retinal degeneration may be observed as a reduction in the rate of retinal degeneration or a cessation of the progression of retinal degeneration, effects which may be observed by, e.g., fundus photography, OCT, or AO, by comparing test results after administration of the composition to test results before administration of the subject composition. As another example, therapeutic efficacy in treating a progressive cone dysfunction may be observed as a reduction in the rate of progression of cone dysfunction, as a cessation in the progression of cone dysfunction, or as an improvement in cone function, effects which may be observed by, e.g., ERG and/or cERG; colour vision tests; functional adaptive optics; and/or visual acuity tests, for example, by comparing test results after administration of the composition to test results before administration of the subject composition and detecting a change in cone viability and/or function.

Individual doses are typically not less than an amount required to produce a measurable effect on the subject, and may be determined based on the pharmacokinetics and pharmacology for absorption, distribution, metabolism, and excretion (“ADME”) of the subject composition or its by-products, and thus based on the disposition of the composition within the subject. This includes consideration of the route of administration as well as dosage amount, which can be adjusted for subretinal (applied directly to where action is desired for mainly a local effect), intravitreal (applied to the vitreous for a pan-retinal effect), or parenteral (applied by systemic routes, e.g. intravenous, intramuscular, etc.) applications. Effective amounts of dose and/or dose regimen can readily be determined empirically from preclinical assays, from safety and escalation and dose range trials, individual clinician-patient relationships.

It will be understood that the invention disclosed and defined in this specification extends to all alternative combinations of two or more of the individual features mentioned or evident from the text or drawings. All of these different combinations constitute various alternative aspects of the invention.

### EXAMPLES

Here the inventors have found an in vitro method for reprogramming Muller glial cells into cone photoreceptors through transduction with a cocktail of transcription factors, termed induced cones (iCones). Furthermore, the inventors have demonstrated how to perform an in vivo method of reprogramming cells in the eye to cone photoreceptors using iCone factors described herein, which may be used to prevent the progressive loss of vision associated with diseases that cause photoreceptor degeneration.

### Example 1—Materials and Methods

### Reporter Cell Generation

To establish a human MG cell line with cone reporter, human MG cells MIO-M1 were transduced with the OPN1LW/MW DsRed reporter lentivirus at a MOI=2, the cells were incubated with the virus overnight in 10% FBS/DMEM medium with 8 μg/mL of polybrene. Two days after transduction, the cells were selected with 2 μg/ml of puromycin (ThermoFisher Scientific, A11138-03) for a period of two days to generate a stable cell line.

### Genome-Wide CRISPRa Screening

The cone reporter MG cells were used for CRISPRa screening using a human CRISPR/Cas9 SAM pooled lentivirus library (LentiSAM v2), consisting of 70,290 sgRNAs targeting 23,430 genes. Reporter MG cells were transduced with the pooled lentiviral library overnight in 10% FBS/DMEM medium with polybrene. Following transduction, the virus was removed and fresh 10% FBS/DMEM medium with TSA (Sigma-Aldrich) was added to the culture. On day 3 after transduction, the medium was replaced by NBM/B27/TSA, which was maintained for the remaining of the reprogramming until day 14.

Flow cytometry sorting was performed on day 14 to isolate DsRed+ iCones using a BD Influx cell sorter (BD Biosciences). DNA extraction was performed on the sample, the sgRNAs were amplified from the pooled gDNA of the DsRed+ cells. The PCR amplification was performed with a Q5 High-Fidelity 2× Master Mix (NEB) and it was monitored with a Fast 7500 Real-Time PCR Systems (Applied Biosystems) in the presence of 1×SYBR Green I (Thermo Fisher Scientific). Afterwards, the PCR product was separated in a 2% (w/v) agarose gel and the sgRNAs were purified with a QIAquick Gel Extraction Kit (QIAGEN). The sgRNA sequences were analysed by Illumina NextSeq 500 (Australian Genome Research Facility). The sgRNA distribution was determined with the python script “count_spacers.py” provided by Joung et al., Nat. Protoc., 2017, 12:828-863.

### Gene Ontology and Network Topology Analysis

Gene ontology was performed using Enrichr (Chen et al., BMC Bioformatics, 2013, 14: 128). For network topology analysis, transcription factors were extracted using DAVID v6.8 (LHRI) (Huang et al., Nature Protoc., 2009, 4(1): 44-57) and network topology analysis was performed using Cytoscape V3.8 (Shannon et al., Genome Research, 2003, 13: 2498-2504).

### Multielectrode Array

The microelectrode array (MEA) recording system (Multichannel Systems) was used to measure the extracellular field potential of iPH cells. MIO-M1 cells were cultured onto MEA plates the day before transfection and recordings were performed 14 days following reprogramming. Data were analyzed with MC Rack software.

In Vitro Reprogramming for iCones

On day −1, 6×10⁴ cells of the Müller glia cell line MIO-M1 were seeded in a 12 well plate and cultured with 10% FBS/DMEM. On day 0, the cells were co-transduced with combinations of the reprogramming transgene lentiviruses and the cone reporter lentivirus (OPN1LW/MW-DsRed). The following day, fresh medium with 10 ng/ml of TSA (Sigma-Aldrich) was added. On day 3, the culture medium was replaced to NBM/B27/TSA/T3 containing Neurobasal medium (NBM), B27 (Thermo Fisher Scientific), 10 ng/ml of TSA and T3, which were maintained until day 14. At day 14, DsRed+ cells were quantified manually to assess the reprogramming efficiency. For characterisation of iCones, RNA was extracted and sent for RNAseq (Australian Genome Research Facility). Briefly, quality of RNA samples were checked using bioanalyzer and transcriptome libraries were prepared using the TruSeq Stranded mRNA kit (Illumina). Subsequently, samples were processed for 100 bp single-end sequencing using an Illumina Novaseq 6000. The human reference transcriptome GRCh38 was used as an index and transcript level quantification was performed using Salmon to obtain gene-level counts. The tximport package was used to import and summarize the gene-level counts, using the lengthScaledTPM function, and data are expressed as transcripts per million (TPM) for gene expression analysis.

In Vivo Delivery of iCone Genes

Intravitreal injection of AAV containing iCone factors were performed in P23H3 rats (LaVail et al., Exp. Eye Res., 2018, 167:56-90), which are characterised by progressive photoreceptor degeneration. P23H-3 rats at 7 weeks of age were injected with AAV (ShH10Y serotype) carrying iCone genes by intravitreal delivery. Briefly, animals were anesthetized with ketamine (Ilium Ketamil, 20 mg/kg subcutaneous or intramuscular) and xylazine (Ilium Xylazil-20, 2 mg/kg subcutaneous). 1% tropicamide was applied to induce mydriasis. A heat pad was used to maintain the animals' body temperature at 37° C. Intravitreal injection was performed to delivery 3 μl AAV into the vitreous cavity in the treated eyes. Untreated eyes were used as naïve control.

### Electroretinogram (ERG) Analysis

Dark-adapted full field electroretinography (ffERG) was performed to assess the retinal function before treatment (baseline) and 4 weeks after treatment. Animals were placed in the dark for 12 hr before recording of the ffERG. Animals were anesthetized with ketamine (20 mg/kg subcutaneous or intramuscular) and xylazine (2 mg/kg subcutaneous), then maintained with ketamine at one-third the original dose as required. Topical application of a sterile saline solution (0.9%) was used to keep the cornea hydrated during assessment. Pupils were dilated with 1% tropicamide and 2.5% phenylephrine, and ocular lubricant (HPMC PAA gel) was applied to prevent corneal desiccation. ffERG was performed using a Espion E2. The retinal response (mean of 3 measurements) was recorded for stimulus intensities from 0.1 to 30 cd·s·m-2. For analysis, the a-wave and b-wave readings after treatment were normalised to the baseline (before treatment) for each individual eye to assess the changes in retinal function following treatment.

### Immunohistochemistry on Retina

At 4 weeks post-treatment, the P23H3 rats were terminated and the posterior eyecups were surgically extracted and fixed in 4% PFA for 2 hours at room temperature. Samples were placed in 10% sucrose for 1 hour then 20% for 1 hour and 30% overnight at 4° C. Eyecups were placed in 1/1 mix of 30% sucrose/OCT for 1 hour the next day then embedded in OCT compound and cryosectioned.

Standard immunostaining procedure was performed as previously described (Wong et al., Stem Cells, 29(10): 1517-27). Briefly, samples were fixed in 4% paraformaldehyde, followed by blocking with 10% goat serum (Sigma) and permeabilization with 0.1% Triton X-100 (Sigma). The samples were then immunostained with antibodies against Recoverin (Millipore), followed by the appropriate Alexa Fluor 488 or 568 secondary antibodies (Abcam), and nuclear counterstain with DAPI (Sigma, 1 ug/ml). Samples were imaged using a Zeiss Axio Vert.A1 fluorescent microscope or a Nikon Eclipse TE2000-U. Specificity of the staining is confirmed by absence of signal in isotype control.

### Example 2—Experimental Setup for Genome-Wide CRISPRa Screening for Genes that Promote Reprogramming of Human Muller Glial (MG) Cells to Cones

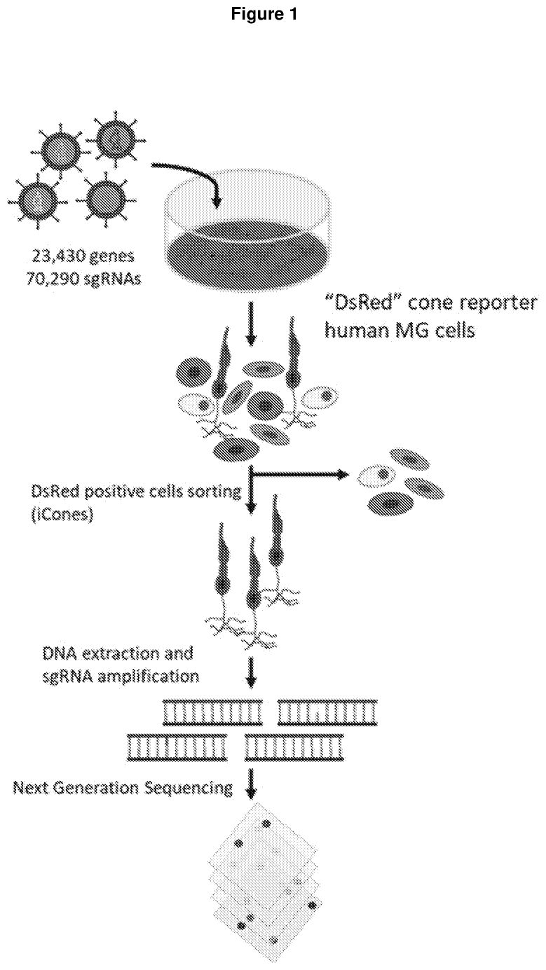

FIG. 1 illustrates the experimental setup for genome-wide CRISPR activation (CRISPRa) screening to identify genes promoting cone reprogramming. To facilitate live monitoring and detection of cell reprogramming, the inventors generated human Muller glial (MG) cells (MIO-M1) carrying a fluorescent reporter for the cone marker L/M opsins (OPN1LW/MW-DsRed), using the promoter region for L/M opsins.

iCone Generation

For iCone generation, MIO-M1 were transduced with lentiviruses containing iCone factors (MOI=10). The virus was incubated overnight in 10% FBS/DMEM medium with 8 μg/mL of polybrene. After the transduction, the virus was removed and fresh 10% FBS/DMEM medium with 10 ng/μL of TSA was added to the cells. On day 3 after transduction, the medium was replaced by NBM with B27 and 10 ng/μL of TSA. For the rest of the reprogramming, the medium was replaced with fresh NBM+B27+10 ng/μL of TSA every two days until day 14

### Example 3—Identification of Genes that Promote iCone Reprogramming

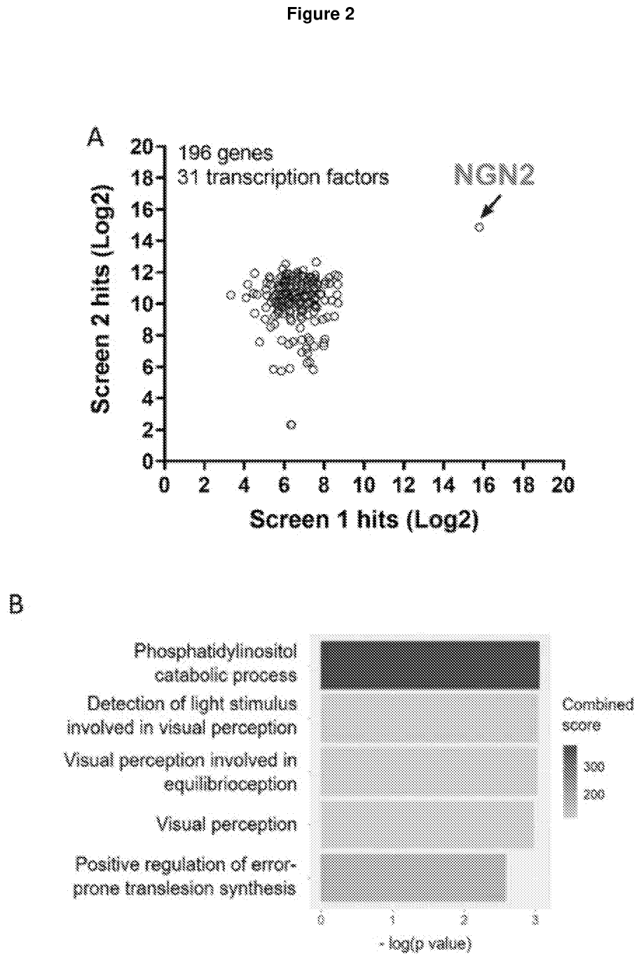

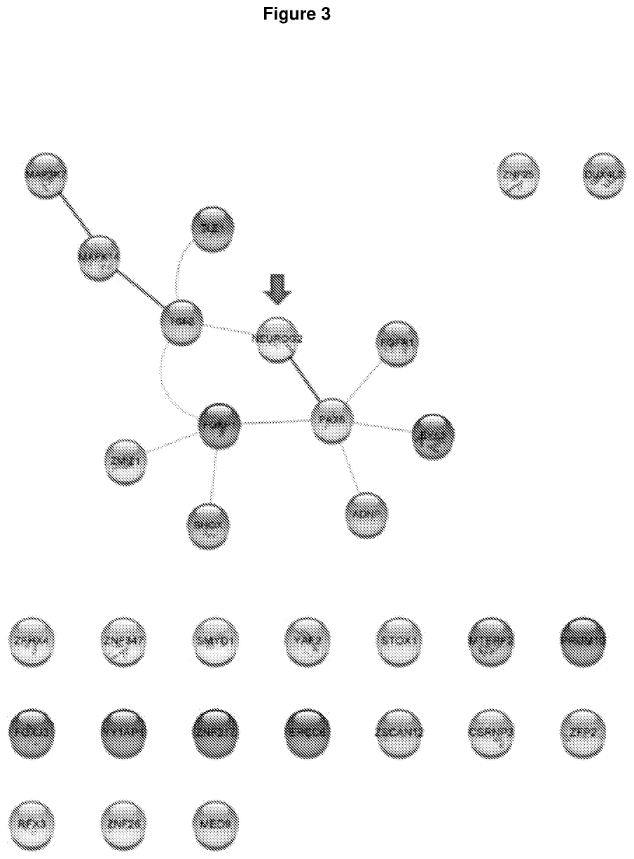

Using the reporter cell line as described above, the inventors performed genome-wide CRISPRa screening to identify genes that reprogram MG cells into cone photoreceptors, termed induced cones (iCones). The reporter MG cells were transduced with pooled lentiviruses carrying the CRISPRa library (SAM library containing 70,290 sgRNAs targeting 23,430 genes). The SAM library and sequences of sgRNA are described in Konermann et al., Nature, 2015, 517(7536): 583-8, the contents of which are incorporated by reference in its entirety. After 14 days, DsRed+ iCones were sorted using flow cytometry and sequenced to identify candidate genes that promote iCone reprogramming. The pilot screenings identified 196 candidate genes, including 31 transcription factors (FIG. 2A). Gene ontology analysis of the candidate genes showed that the top 4 biological processes are related to phototransduction and visual perception, which support their roles in iCone reprogramming (FIG. 2B). Notably, the top hit NEUROG2 (also referred to as NGN2) is a master transcription factor in neural development and is significantly over-represented compared to other candidate genes. Network topology analysis showed a key transcriptional network among the candidate genes, including NEUROG2 as a core factor (FIG. 3). Taking these candidate genes and a list of transcription factors with known roles in retinal development, the inventors shortlisted 12 transcription factors for further reprogramming tests: CRX (C), MEF2C (M), THRB (T), RAX (R), NEUROD1 (N), RORA (Roa), OTX2 (O), PAX6 (P), FOXP1 (F), ASCL1 (A), NEUROG2 (Ng), ONECUT1 (On).

### Example 4—Secondary Screening of Transcription Factor Cocktails for iCone Reprogramming and Photoreceptor Characterisation

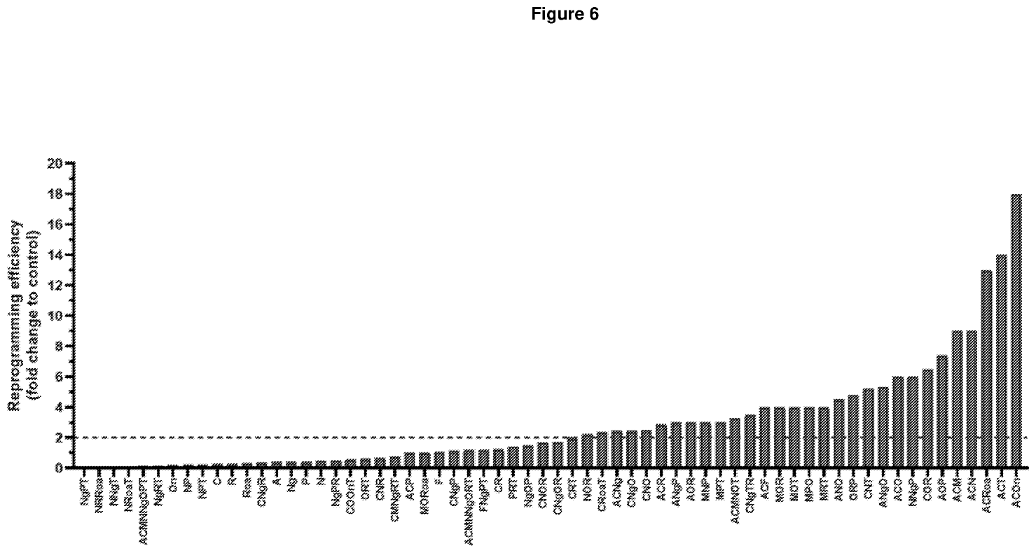

Next, the inventors shortlisted 12 transcription factors with conserved roles in retinal/neural development and performed an initial screen of iCone reprogramming. Using the CRISPR activation system as described above the inventors induced expression of the 12 transcription factors in “cocktails”. The inventors identified several factor cocktails that successfully promoted iCones reprogramming within 2 weeks (FIG. 6; Table 3).

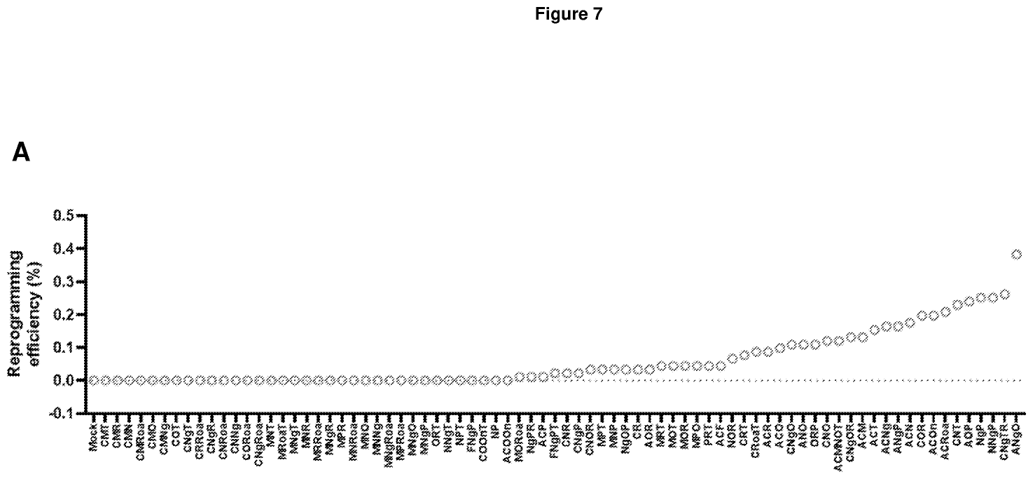

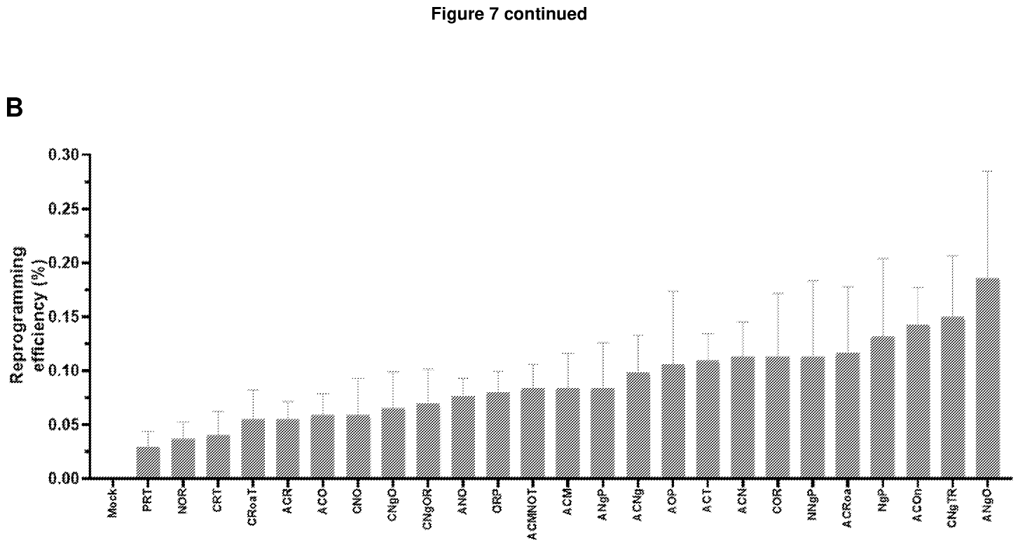

To confirm these findings the inventors performed another primary screen using a lentiviral transgene system to overexpress the cocktails of the transcription factors and subsequently validated the top hits in secondary screens (FIG. 7). These screenings have identified multiple factor cocktails that can successfully promote iCones reprogramming (FIG. 7B).

TABLE 3
Transcription factor cocktails for iCone reprogramming
with 2-fold increase compared to control.

| Cocktail | Reprogramming efficiency (mean |
|---|---|
| abbreviation | fold change compared to control) |
| CRT | 2 |
| NOR | 2.222353 |
| CRoaT | 2.345588 |
| ACNg | 2.471053 |
| CNgO | 2.471053 |
| CNO | 2.5 |
| ACR | 2.870175 |
| ANgP | 3 |
| AOR | 3 |
| MNP | 3 |
| MPT | 3 |
| ACMNOT | 3.2746 |
| CNgTR | 3.469736 |
| ACF | 4 |
| MOR | 4 |
| MOT | 4 |
| MPO | 4 |
| MRT | 4 |
| ANO | 4.515789 |
| ORP | 4.807018 |
| CNT | 5.25 |
| ANgO | 5.333333 |
| ACO | 6 |
| NNgP | 6 |
| COR | 6.5 |
| AOP | 7.381579 |
| ACM | 9 |
| ACN | 9 |
| ACRoa | 13 |
| ACT | 14 |
| ACOn | 18 |

Ng: NEUROG2,
C: CRX,
R: RAX,
Roa: RORA,
N: NEUROD1,
O: OTX2,
A: ASCL1,
P: PAX6,
T: THRB,
M: MEF2C,
F: FOXP1,
On: ONECUT1

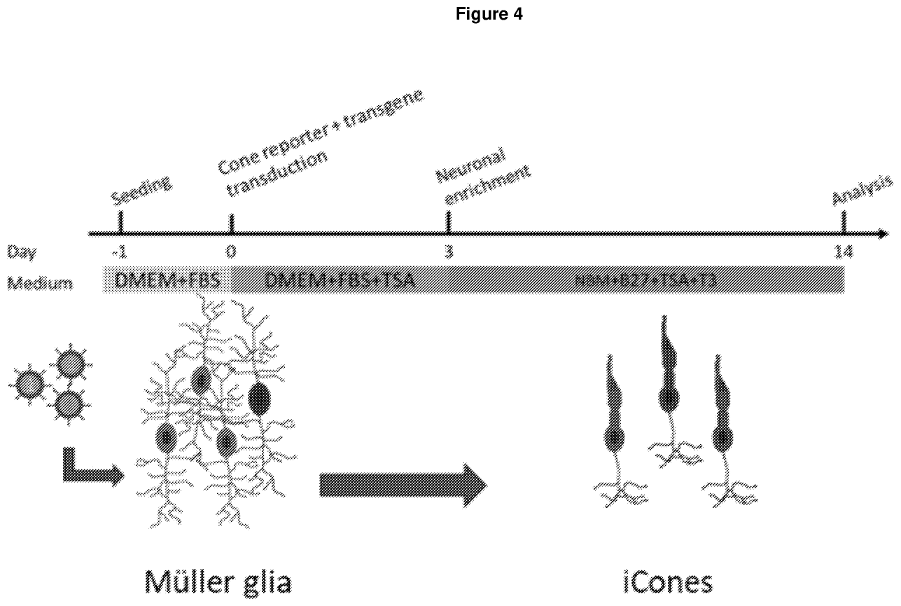

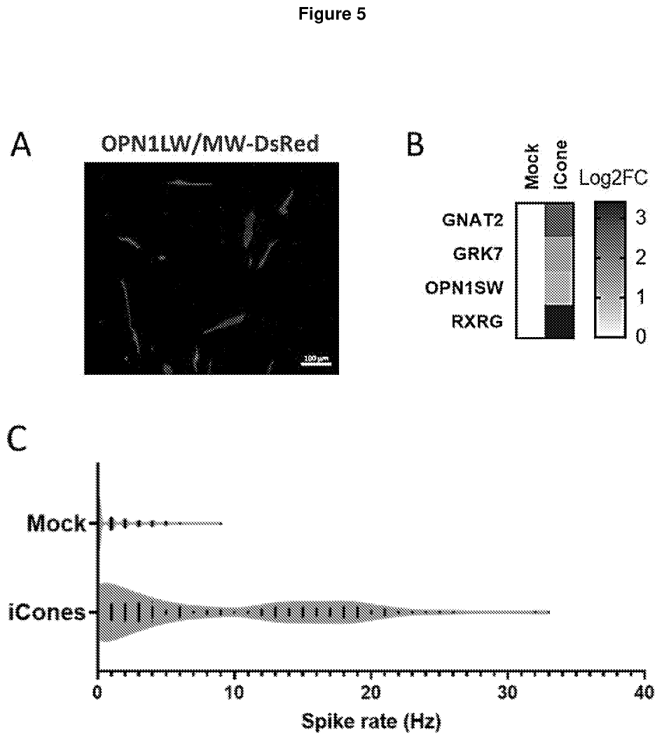

The inventors performed screening with different factor cocktails to promote iCone reprogramming (FIG. 4). The inventors demonstrated that all 12 candidate iCone genes could be successfully expressed in MG cells using a lentiviral system (data not shown). Characterisation of the iCones showed expression of a panel of cone marker genes, including OPN1L/MW, GNAT2, GRK7, OPN1SW and RXRG (FIG. 5A&5B). These results provide the evidence for the feasibility of using cell reprogramming to convert human MG cells into iCones in vitro.

Furthermore, multielectrode array analysis showed that the iCones possess functional electrophysiology (FIG. 5C). These results provide the first evidence for the feasibility of using direct reprogramming human MG cells to generate cones in vitro.

### Example 5—Reprogramming Retinal Cells to Cone Photoreceptor Cells In Vivo Using iCone Factors

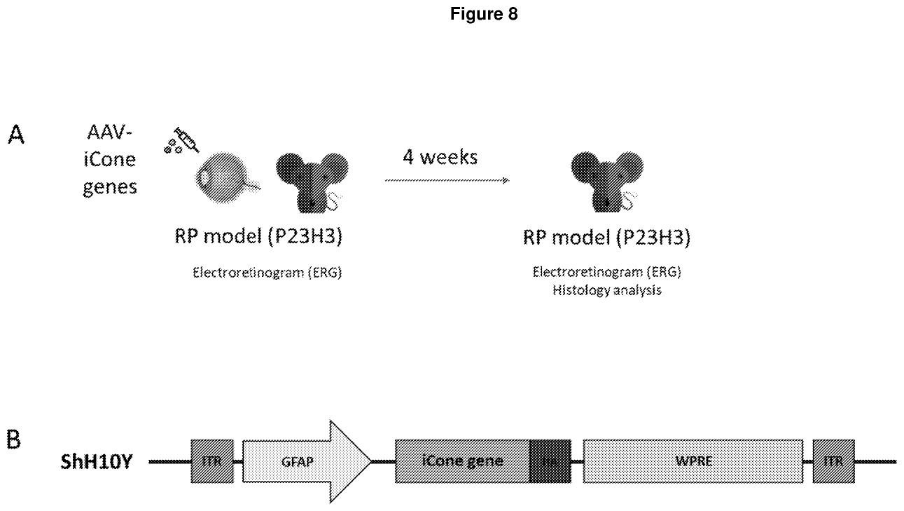

The inventors tested the ability of using iCone factors to prevent vision loss in a rat photoreceptor degeneration model. P23H-3 is a well-established rat retinitis pigmentosa (RP) model caused by a rhodopsin mutation (as described in LaVail et al., Exp Eye Res, 2018; 167:56-90). P23H-3 undergoes a gradual photoreceptor loss (cones and rods) characteristic of human autosomal dominant RP, making it an ideal model to evaluate iCone reprogramming (FIG. 8A) and allowing clinical translation to RP patients.

The inventors utilised the adeno-associated viruses (AAV) as a delivery system to target the Muller glia (MG) cells in the retina (FIG. 8B). To ensure specific targeting of MG cells in vivo, the inventors utilised the MG-specific AAV serotype ShH10Y by intravitreal delivery, coupled with the MG-specific promoter GFAP to drive expression of iCone genes. The inventors performed viral delivery of a set of iCone factors (ACNg: Ascl1, Crx, Ngn2) by intravitreal injection into P23H rats, and analysed the rats visual function using electroretinogram (ERG) before and after treatment for 4 weeks.

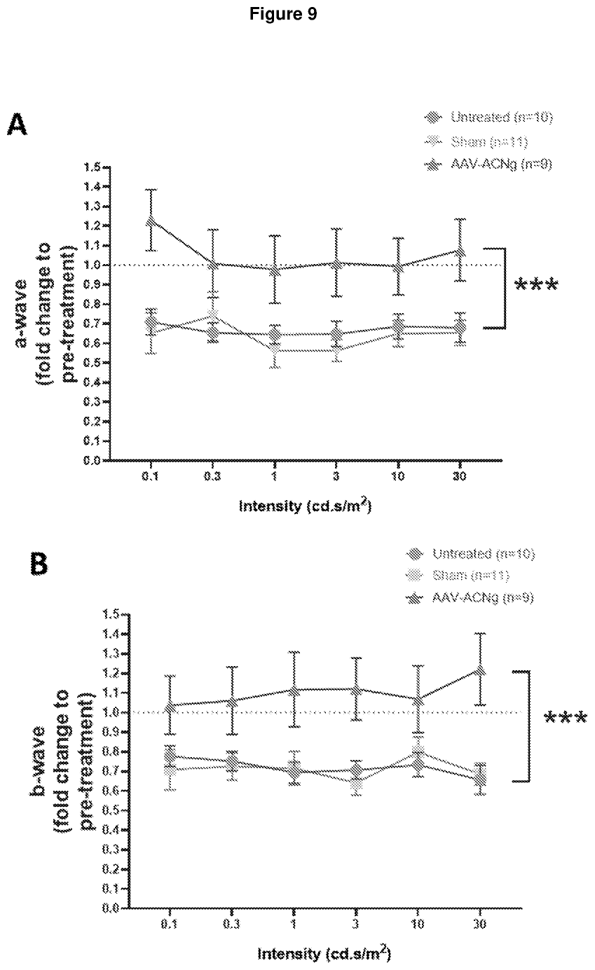

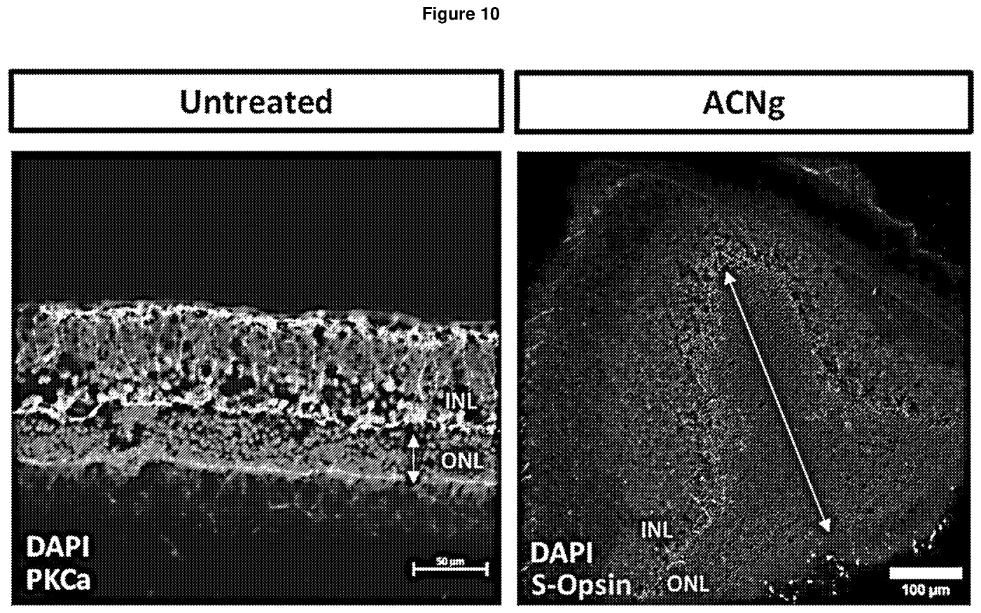

At 4 weeks post-treatment, electroretinogram (ERG) analysis showed that AAV delivery of iCone factors ACNg to the retina improved visual responses in P23H-3 rats (FIG. 9). Also, the inventors observed localised increase in the photoreceptor layer thickness (outer nuclear layer, ONL) in P23H3 rats treated with iCone factors compared to sham controls (FIG. 10). Altogether, these results support the use of in vivo reprogramming as a novel therapeutic approach to treat photoreceptor degeneration and rescue vision.

1. A method for reprogramming a source cell, the method comprising increasing the protein expression of one or more transcription factors, or biologically active fragments or variants thereof, in the source cell, wherein the source cell is reprogrammed to exhibit at least one characteristic of a target cell, wherein:
the source cell is a glial cell;
the target cell is a photoreceptor cell; and
the transcription factors are one or more of those selected from NEUROG2, CRX, RAX, RORA, NEUROD1, OTX2, ASCL1, PAX6, THRB, MEF2C, FOXP1 and ONECUT1.

2. An in vitro method of generating a cell exhibiting at least one characteristic of a photoreceptor cell from a source cell, the method comprising:
increasing the amount of one or more transcription factors, or biologically active fragments or variant thereof, in the source cell; and
culturing the source cell for a sufficient time and under conditions to allow differentiation to a photoreceptor cell; thereby generating the cell exhibiting at least one characteristic of a photoreceptor cell from a source cell, wherein:
the source cell is a glial cell,
the transcription factors are one or more of those selected from NEUROG2, CRX, RAX, RORA, NEUROD1, OTX2, ASCL1, PAX6, THRB, MEF2C, FOXP1 and ONECUT1.

3. An in vitro method for reprogramming a source cell to a cell that exhibits at least one characteristic of a photoreceptor cell, the method comprising:
providing a source cell, or a cell population comprising a source cell;
transfecting said source cell with one or more nucleic acids comprising a nucleotide sequence that encodes one or more transcription factors; and
culturing said cell or cell population, and optionally monitoring the cell or cell population for at least one characteristic of a photoreceptor cell, wherein:
the source cell is a glial cell and
the transcription factors are one or more of those selected from NEUROG2, CRX, RAX, RORA, NEUROD1, OTX2, ASCL1, PAX6, THRB, MEF2C, FOXP1 and ONECUT1.

4. An in vitro method for reprogramming a source cell to a cell that exhibits at least one characteristic of a photoreceptor cell, the method comprising:
providing a source cell, or a cell population comprising a source cell;
transfecting said source cell with one or more nucleic acids for increasing the expression of one or more genes encoding one or more transcription factors; and
culturing said cell or cell population, and optionally monitoring the cell or cell population for at least one characteristic of a photoreceptor cell, wherein:
the source cell is a glial cell and
the transcription factors are one or more of those selected from NEUROG2, CRX, RAX, RORA, NEUROD1, OTX2, ASCL1, PAX6, THRB, MEF2C, FOXP1 and ONECUT1.

5. A method of claim 4, wherein the one or more nucleic acids comprise sgRNAs for use in a CRISPR activation system for increasing expression of genes encoding the one or more transcription factors.

6. A method of any one of claims 3 to 5, wherein the method comprises transfecting the source cell with nucleic acids encoding or for increasing the expression of least two of: NEUROG2, CRX, RAX, RORA, NEUROD1, OTX2, ASCL1, PAX6, THRB, MEF2C, FOXP1 and ONECUT1; at least three of NEUROG2, CRX, RAX, RORA, NEUROD1, OTX2, ASCL1, PAX6, THRB, MEF2C, FOXP1 and ONECUT1; at least four of NEUROG2, CRX, RAX, RORA, NEUROD1, OTX2, ASCL1, PAX6, THRB, MEF2C, FOXP1 and ONECUT1; at least five of NEUROG2, CRX, RAX, RORA, NEUROD1, OTX2, ASCL1, PAX6, THRB, MEF2C, FOXP1 and ONECUT1; at least six of NEUROG2, CRX, RAX, RORA, NEUROD1, OTX2, ASCL1, PAX6, THRB, MEF2C, FOXP1 and ONECUT1.

7. A method of any one of claims 1 to 5, wherein the transcription factors are one or more of those selected from NEUROG2, CRX, RAX, RORA, NEUROD1, OTX2, ASCL1, PAX6, THRB, MEF2C, FOXP1 and ONECUT1, wherein the combination results in a photoreceptor, or photoreceptor-like, cell with a fold increase in opsin mRNA expression of equal to, or greater than, 2, 3, 4, 5, 6, 7, 8, 9, 10, 15, 20, 25, 30, 35, 40, 45, 50, 55, 60, 65, 70 or 75 fold compared to the opsin expression in the source cell type.

8. A method of claim 7, wherein the fold increase is equal to, or greater than, 4, 5, 6, 7, 8, 9, 10, 15, 20, 25, 30, 35, 40, 45, 50, 55, 60, 65, 70 or 75 fold compared to the opsin expression in the source cell type.

9. A method of claim 8, wherein the fold increase is equal to, or greater than, 10, 15, 20, 25, 30, 35, 40, 45, 50, 55, 60, 65, 70 or 75 fold compared to the opsin expression in the source cell type.

10. A method of any one of claims 1 to 9, wherein the source cell is a glial cell, and the transcription factors, biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2;
(k) CRX, NEUROD1 and THRB;
(l) OTX2, RAX, and PAX6;
(m) ASCL1, NEUROD1, and OTX2;
(n) MEF2C, RAX and THRB;
(o) MEF2C, PAX6 and OTX2;
(p) MEF2C, OTX2 and THRB;
(q) MEF2C, OTX2 and RAX;
(r) ASCL1, CRX and FOXP1;
(s) CRX, NEUROG2, THRB and RAX;
(t) ASCL1, CRX, MEF2C, NEUROD1, OTX2 and THRB;
(u) MEF2C, PAX6 and THRB;
(v) MEF2C, NEUROD1 and PAX6;
(w) ASCL1, OTX2 and RAX;
(x) ASCL1, NEUROG2 and PAX6;
(y) ASCL1, CRX and RAX;
(z) CRX, NEUROD1 and OTX2;
(aa) CRX, NEUROG2 and OTX2;
(bb) ASCL1, CRX and NEUROG2;
(cc) CRX, RORA and THRB;
(dd) NEUROD1, OTX2 and RAX;
(ee) CRX, RAX and THRB;
(ff) MEF2C, OTX2 and RORA;
(gg) NEUROG2, PAX6 and RAX;
(hh) ASCL1, CRX and PAX6;
(ii) FOXP1, NEUROG2, PAX6 and THRB;
(jj) CRX, NEUROD1 and RAX;
(kk) CRX, NEUROG2 and PAX6;
(ll) CRX, NEUROD1, OTX2 and RAX;
(mm) NEUROG2, OTX2 and PAX6;
(nn) CRX and RAX;
(oo) PAX6 and RAX;
(pp) CRX, NEUROG2, OTX2 and RAX; or
(bb) NEUROG2 and PAX6.

11. A method of any one of claims 1 to 9, wherein the source cell is a glial cell, and the transcription factors, biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2;
(k) OTX2, RAX, and PAX6;
(l) ASCL1, NEUROD1, and OTX2;
(m) MEF2C, RAX and THRB;
(n) MEF2C, PAX6 and OTX2;
(o) MEF2C, OTX2 and THRB;
(p) MEF2C, OTX2 and RAX;
(q) ASCL1, CRX and FOXP1;
(r) CRX, NEUROG2, THRB and RAX;
(s) ASCL1, CRX, MEF2C, NEUROD1, OTX2 and THRB;
(t) MEF2C, PAX6 and THRB;
(u) MEF2C, NEUROD1 and PAX6;
(v) ASCL1, OTX2 and RAX;
(w) ASCL1, NEUROG2 and PAX6;
(x) ASCL1, CRX and RAX;
(y) CRX, NEUROD1 and OTX2;
(z) CRX, NEUROG2 and OTX2;
(aa) ASCL1, CRX and NEUROG2;
(bb) CRX, RORA and THRB;
(cc) NEUROD1, OTX2 and RAX;
(dd) CRX, RAX and THRB;
(ee) MEF2C, OTX2 and RORA;
(ff) NEUROG2, PAX6 and RAX;
(gg) ASCL1, CRX and PAX6;
(hh) FOXP1, NEUROG2, PAX6 and THRB;
(ii) CRX, NEUROD1 and RAX;
(jj) CRX, NEUROG2 and PAX6;
(kk) CRX, NEUROD1, OTX2 and RAX;
(ll) NEUROG2, OTX2 and PAX6;
(mm) CRX and RAX;
(nn) PAX6 and RAX;
(oo) CRX, NEUROG2, OTX2 and RAX; or
(pp) NEUROG2 and PAX6.

12. A method of any one of claims 1 to 9, wherein the source cell is a glial cell, and the transcription factors, biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2;
(k) CRX, NEUROD1 and THRB;
(l) OTX2, RAX, and PAX6;
(m) ASCL1, NEUROD1, and OTX2;
(n) MEF2C, RAX and THRB;
(o) MEF2C, PAX6 and OTX2;
(p) MEF2C, OTX2 and THRB;
(q) MEF2C, OTX2 and RAX;
(r) ASCL1, CRX and FOXP1;
(s) CRX, NEUROG2, THRB and RAX;
(t) ASCL1, CRX, MEF2C, NEUROD1, OTX2 and THRB;
(u) MEF2C, PAX6 and THRB;
(v) MEF2C, NEUROD1 and PAX6;
(w) ASCL1, OTX2 and RAX;
(x) ASCL1, NEUROG2 and PAX6;
(y) ASCL1, CRX and RAX;
(z) CRX, NEUROD1 and OTX2;
(aa) CRX, NEUROG2 and OTX2;
(bb) ASCL1, CRX and NEUROG2;
(cc) CRX, RORA and THRB;
(dd) NEUROD1, OTX2 and RAX; or
(qq) CRX, RAX and THRB.

13. A method of any one of claims 1 to 9, wherein the source cell is a glial cell, and the transcription factors, biologically active fragments or variants thereof, are:
(a) ASCL1, NEUROG2 and OTX2;
(b) CRX, NEUROG2, THRB and RAX;
(c) NEUROD1, NEUROG2 and PAX6;
(d) NEUROG2 and PAX6;
(e) ASCL1, OTX2 and PAX6;
(f) CRX, NEUROD1 and THRB;
(g) ASCL1, CRX and RORA;
(h) ASCL1, CRX and ONECUT1;
(i) CRX, OTX2 and RAX;
(j) ASCL1, CRX and NEUROD1;
(k) ASCL1, NEUROG2 and PAX6;
(l) ASCL1, CRX and NEUROG2;
(m) ASCL1, CRX and THRB;
(n) ASCL1, CRX and MEF2C;
(o) CRX, NEUROG2, OTX2 and RAX;
(p) ASCL1, CRX, MEF2C, OTX2, and THRB;
(q) CRX, NEUROD1 and OTX2;
(r) OTX2; RAX and PAX6;
(s) ASCL1; NEUROD1 and OTX2;
(t) CRX; NEUROG2 and OTX2;
(u) ASCL1; CRX and OTX2;
(v) ASCL1, CRX and RAX;
(w) CRX, RORA and THRB;
(x) CRX, RAX and THRB; or
(y) NEUROD1; OTX2 and RAX.

14. A method of any one of claims 1 to 9, wherein the source cell is a glial cell, and the transcription factors, biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2;
(k) CRX, NEUROD1 and THRB;
(l) OTX2, RAX, and PAX6;
(m) ASCL1, NEUROD1, and OTX2;
(n) MEF2C, RAX and THRB;
(o) MEF2C, PAX6 and OTX2;
(p) MEF2C, OTX2 and THRB;
(q) MEF2C, OTX2 and RAX;
(r) ASCL1, CRX and FOXP1;
(s) CRX, NEUROG2, THRB and RAX;
(t) ASCL1, CRX, MEF2C, NEUROD1, OTX2 and THRB;
(u) MEF2C, PAX6 and THRB;
(v) MEF2C, NEUROD1 and PAX6;
(w) ASCL1, OTX2 and RAX; or
(x) ASCL1, NEUROG2 and PAX6.

15. A method of any one of claims 1 to 9, wherein the source cell is a glial cell, and the transcription factors, biologically active fragments or variants thereof, are:
(a) ASCL1, NEUROG2 and OTX2;
(b) CRX, NEUROG2, THRB and RAX;
(c) NEUROD1, NEUROG2 and PAX6;
(d) NEUROG2 and PAX6;
(e) ASCL1, OTX2 and PAX6;
(f) CRX, NEUROD1 and THRB;
(g) ASCL1, CRX and RORA;
(h) ASCL1, CRX and ONECUT1;
(i) CRX, OTX2 and RAX;
(j) ASCL1, CRX and NEUROD1;
(k) ASCL1, NEUROG2 and PAX6;
(l) ASCL1, CRX and NEUROG2; or
(m) ASCL1, CRX and THRB.

16. A method of any one of claims 1 to 9, wherein the source cell is a glial cell, and the transcription factors, biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2;
(k) CRX, NEUROD1 and THRB;
(l) OTX2, RAX, and PAX6;
(m) ASCL1, NEUROD1, and OTX2;
(n) MEF2C, RAX and THRB;
(o) MEF2C, PAX6 and OTX2;
(p) MEF2C, OTX2 and THRB;
(q) MEF2C, OTX2 and RAX; or
(r) ASCL1, CRX and FOXP1.

17. A method of any one of claims 1 to 9, wherein the source cell is a glial cell, and the transcription factors, biologically active fragments or variants thereof, are:
(a) ASCL1, NEUROG2 and OTX2;
(b) CRX, NEUROG2, THRB and RAX;
(c) NEUROD1, NEUROG2 and PAX6;
(d) NEUROG2 and PAX6;
(e) ASCL1, OTX2 and PAX6; or
(f) CRX, NEUROD1 and THRB.

18. A method of any one of claims 1 to 9, wherein the source cell is a glial cell, and the transcription factors, biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6;
(i) ASCL1, CRX and OTX2;
(j) ASCL1, NEUROG2, and OTX2; or
(k) CRX, NEUROD1 and THRB.

19. A method of any one of claims 1 to 9, wherein the source cell is a glial cell, and the transcription factors, biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C;
(f) ASCL1, OTX2 and PAX6;
(g) CRX, OTX2 and RAX;
(h) NEUROD1, NEUROG2 and PAX6; or
(i) ASCL1, CRX and OTX2.

20. A method of any one of claims 1 to 9, wherein the source cell is a glial cell, and the transcription factors, biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1;
(e) ASCL1, CRX, and MEF2C; or
(f) ASCL1, OTX2 and PAX6.

21. A method of any one of claims 1 to 9, wherein the source cell is a glial cell, and the transcription factors, biologically active fragments or variants thereof, are:
(a) ASCL1, CRX and ONECUT1;
(b) ASCL1, CRX and THRB;
(c) ASCL1, CRX and RORA;
(d) ASCL1, CRX and NEUROD1; or
(e) ASCL1, CRX, and MEF2C.

22. A method of any one of claims 1 to 9, wherein the source cell is a glial cell, and the transcription factors, biologically active fragments or variants thereof, are:
(a) ASCL1, CRX and ONECUT1;
(b) ASCL1, CRX and THRB; or
(c) ASCL1, CRX and RORA.

23. A method of any one of claims 1 to 9, wherein the source cell is a glial cell, and the transcription factors, biologically active fragments or variants thereof, are:
(a) ASCL1, NEUROG2 and OTX2; or
(b) CRX, NEUROG2, THRB and RAX.

24. A method of any one of claims 1 to 9, wherein the source cell is a glial cell, and the transcription factors, biologically active fragments or variants thereof, are:
(a) ASCL1, CRX and ONECUT1; or
(b) ASCL1, CRX and THRB.

25. A method of any one of claims 1 to 24, wherein the nucleic acid encodes for one or more of the transcription factors listed in Table 1.

26. A method of any one of claims 1 to 25, wherein the source cell is a human cell.

27. A method of any one of claims 1 to 26, wherein the glial cell is selected from the group consisting of a Müller glial (MG) cell, an astrocyte and a microglia

28. A method of claim 27, wherein the source cell is a Müller glial cell.

29. A method of any one of claims 2 to 6, wherein culturing the source cell for a sufficient time and under conditions to allow differentiation to a photoreceptor cell including culturing the cells for at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days in a relevant medium as shown in Table 2.

30. A method of any one of claims 1 to 29, wherein the method further comprises the step of expanding the cells exhibiting at least one characteristic of a photoreceptor cell to increase the proportion of cells in the population exhibiting at least one characteristic of a photoreceptor cell.

31. A method of any one of claims 1 to 30, wherein the method further comprises the step of administering the cells, or cell population including a cell, exhibiting at least one characteristic of a target cell type, to an individual.

32. A cell exhibiting at least one characteristic of a photoreceptor cell produced by a method of any one of claims 1 to 31.

33. The cell of claim 32, wherein the cell comprises at least one characteristic of a cone cell.

34. A population of cells, wherein at least 1% of cells exhibit at least one characteristic of a photoreceptor cell and those cells are produced by a method of any one of claims 1 to 33.

35. A population of cells of claim 43, wherein at least 0.01%, at least 0.02%, at least 0.03%, at least 0.04%, at least 0.05, at least 0.06%, at least 0.07%, at least 0.08%, at least 0.09%, at least 0.1%, at least 0.15%, at least 0.2%, at least 0.25%, at least 0.3%, at least 0.35%, at least 0.4%, at least 0.45%, at least 0.5, at least 0.6%, at least 0.7%, at least 0.8%, at least 0.9%, at least 1%, at least 2%, at least 3%, at least 4%, at least 5% at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or at least 100% of the cells in the population exhibit at least one characteristic of a photoreceptor cell.

36. A kit for producing a cell exhibiting at least one characteristic of a photoreceptor cell, the kit comprising one or more nucleic acids having one or more nucleic acid sequences encoding a transcription factor selected from NEUROG2, CRX, RAX, RORA, NEUROD1, OTX2, ASCL1, PAX6, THRB, MEF2C, FOXP1 and ONECUT1.

37. A kit for producing a cell exhibiting at least one characteristic of a photoreceptor cell, the kit comprising one or more nucleic acids, optionally sgRNAs, for increasing the expression of one or more genes encoding a transcription factor selected from NEUROG2, CRX, RAX, RORA, NEUROD1, OTX2, ASCL1, PAX6, THRB, MEF2C, FOXP1 and ONECUT1.

38. A kit of claim 36 or 37, wherein the transcription factors are one or more of the specific combinations defined in (a) to (qq) of claim 10.

39. A kit of any one of claims 36 to 38, wherein the kit further comprises instructions for reprogramming a source cell to a cell exhibiting at least one characteristic of a photoreceptor cell according to any one of the methods of claims 1 to 25.

40. A kit of any one of claims 36 to 38, when used in a method of any one of claims 1 to 25.

41. A method of treating a condition associated with or caused by degeneration of photoreceptor cells in an individual in need thereof, the method comprising administering to the individual a cell of claim 32 or 33 or cell population of claim 34 or 35.

42. Use of a cell of claim 32 or 33 or cell population of claim 34 or 35 in the manufacture of a medicament for the treatment of a condition associated with or caused by degeneration of photoreceptor cells in an individual in need thereof.

43. A cell of claim 32 or 33 or cell population of claim 34 or 35, for use in the treatment of a condition associated with or caused by degeneration of photoreceptor cells in an individual in need thereof.

44. A method of claim 41, use of claim 42, or cell or cell population of claim 43, wherein the condition associated with or cause by degeneration of photoreceptor cells is any one of retinitis pigmentosa, age-related macular degeneration, choroideremia and diabetic retinopathy.

45. A nucleic acid comprising a nucleotide sequence encoding one or more of the transcription factors defined in any one of claims 1 to 24.

46. A nucleic acid comprising a nucleotide sequence encoding one or more of the sets of transcription factors defined in claim 10.

47. A vector comprising a nucleic acid of claim 45 or 46.

48. An in vitro or ex vivo cell comprising a nucleic acid of claim 45 or 46, or vector of claim 47.

49. A CRISPR activation system for increasing the expression one or more sets of transcription factors defined in claim 10

50. A CRISPR activation system of claim 49 comprising sgRNAs that target genes selected from the group consisting of: CRX, MEF2C, THRB, RAX, NEUROD1, RORA, OTX2, NEUROG2/NGN2, PAX6, FOXP1, ASCL1 and ONECUT1.

51. A nucleic acid or vector of claims 45 to 46, further comprising an expression construct.

52. A nucleic acid or vector of claim 51, wherein the expression construct comprises one or more features of an AAV vector.

53. A nucleic acid or vector of claim 51, wherein the expression construct comprises a promoter.

54. A nucleic acid or vector of claim 53, wherein the promoter is a ubiquitous promoter or a retinal glial cell-specific promoter.

55. A nucleic acid or vector of claim 53, wherein the promoter is the CAG promoter.

56. A nucleic acid or vector of claim 55, wherein the CAG promoter comprises the cytomegalovirus (CMV) early enhancer element, the promoter, the first exon and the first intron of chicken beta-actin (CBA) gene and the splice acceptor of the rabbit beta-globin gene.

57. A nucleic acid or vector of claim 53, wherein the promoter is the GFAP, GLAST or RLBP1 promoter.

58. A nucleic acid or vector of any one of claims 51 to 57, wherein the expression construct further comprises a nucleotide sequence encoding a Kozak sequence.

59. A nucleic acid or vector of any one of claims 51 to 58, wherein the expression construct further comprises a nucleotide sequence encoding a Woodchuck Hepatitis Virus Post-transcriptional Regulatory Element (WPRE).

60. A nucleic acid or vector of any one of claims 51 to 58, wherein the expression construct further comprises a nucleotide sequence encoding a bovine growth hormone (bGH) polyA tail.

61. A nucleic acid or vector of any one of claims 51 to 60, wherein the expression construct further comprises AAV Inverted Terminal Repeats (ITRs).

62. An adeno-associated viral (AAV) vector comprising a nucleotide sequence encoding one or more of the transcription factors defined in any one of claims 1 to 24, or encoding one or more of the sets of transcription factors defined in claim 10, or biologically active fragments or variants thereof.

63. An AAV vector according to claim 62, wherein the nucleotide sequence encoding one or more of the transcription factors defined in any one of claims 1 to 24, or encoding one or more of the sets of transcription factors defined in claim 10 is flanked by two AAV Inverted Terminal Repeats (ITRs).

64. An AAV vector according to claim 62 or 63, wherein the vector is recombinant, synthetic, purified, or substantially purified.

65. A recombinant adeno-associated virus (rAAV) comprising:
(i) an AAV capsid protein; and
(ii) an AAV vector of claims 62 to 64.

66. A pharmaceutical composition comprising a nucleic acid of claims 45, 46 or 51 to 61, vector of claim 47, or AAV vector of 62 to 64, or a recombinant AAV of claim 64, and a pharmaceutically acceptable carrier, diluent or excipient.

67. A cell comprising:
(i) a first vector encoding one of more adeno-associated virus rep protein and/or one or more adeno-associated virus cap protein; and
(ii) a second vector comprising a nucleotide sequence encoding one or more of the transcription factors defined in any one of claims 1 to 24, or encoding one or more of the sets of transcription factors defined in claim 10, or biologically active fragments or variants thereof.

68. A method of decreasing progression of or ameliorating vision loss associated with cone dystrophy in a subject, the method comprising administering to the subject a nucleic acid of claims 45, 46 or 51 to 61, vector of claim 47, AAV vector of 62 to 64, or a recombinant AAV of claim 65, or a pharmaceutical composition of claim 66, thereby of decreasing progression of or ameliorating vision loss associated with or caused by degeneration, or loss, of cone photoreceptor cells.

69. Use of a nucleic acid of claims 45, 46 or 51 to 61, vector of claim 47, AAV vector of 62 to 64, or a recombinant AAV of claim 65, or a pharmaceutical composition of claim 66, in the manufacture of a medicament for decreasing progression of or ameliorating vision loss associated with or caused by degeneration, or loss, of cone photoreceptor cells in a subject.

70. A nucleic acid of claims 45, 46 or 51 to 61, vector of claim 47, AAV vector of 62 to 64, or a recombinant AAV of claim 65, or a pharmaceutical composition of claim 66, for use in decreasing progression of or ameliorating vision associated with or caused by degeneration, or loss, of cone photoreceptor cells in a subject.

71. A method, use or nucleic acid of claims 68 to 70, wherein the subject is a human.

72. A method, use or nucleic acid of claims 68 to 70, wherein the condition associated with or caused by degeneration, or loss, of cone photoreceptor cells is a cone cell disorder.

73. A method, use or nucleic acid of claim 72, wherein the cone cell disorder is a colour vision disorder.

74. A method, use or nucleic acid of claim 73, wherein the colour vision disorder is selected from the group consisting of achromatopsia, blue cone monochromacy, a protan defect, a deutan defect, and a tritan defect.

75. A method, use or nucleic acid of claim 72, wherein the cone cell disorder is a macular dystrophy.

76. A method, use or nucleic acid of claim 75, wherein the macular dystrophy is selected from the group consisting of Stargardt's macular dystrophy, cone dystrophy, Spinocerebellar ataxia type 7, and Bardet-Biedl syndrome-1.

77. A method, use or nucleic acid of claim 72, wherein the cone cell disorder is a vision disorder of the central macula.

78. A method, use or nucleic acid of claim 77, wherein the vision disorder of the central macula is selected from the group consisting of age-related macular degeneration, macular telangiectasia, retinitis pigmentosa, diabetic retinopathy, retinal vein occlusions, glaucoma, choroideremia, Sorsby's fundus dystrophy, adult vitelliform macular dystrophy, Best's disease, Leber's congenital amaurosis, and X-linked retinoschisis.

79. A method, use or nucleic acid of any one of claims 6 to 78, wherein the nucleic acid, AAV vector, a recombinant AAV, or a pharmaceutical composition is administered to the subject by retinal injection into an affected eye of said subject.

80. A method, use or nucleic acid of any one of claims 6 to 78, wherein the nucleic acid, AAV vector, a recombinant AAV, or a pharmaceutical composition is administered to the subject by intravitreal injection into an affected eye of said subject.

81. A method, use or nucleic acid of any one of claims 6 to 78, wherein the nucleic acid, AAV vector, a recombinant AAV, or a pharmaceutical composition is administered to the subject by subretinal injection into an affected eye of said subject.


SEQUENCE LISTING (17 sequences)

SEQ ID NO: 1 - DNA, 245 bp, synthetic construct
  source 1..245: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 2 - DNA, 681 bp, synthetic construct
  source 1..681: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 3 - DNA, 408 bp, synthetic construct
  source 1..408: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 4 - DNA, 10 bp, synthetic construct
  source 1..10: mol_type = other DNA
ggcaccatgg

SEQ ID NO: 5 - DNA, 593 bp, synthetic construct
  source 1..593: mol_type = other DNA
atcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcccc

SEQ ID NO: 6 - DNA, 269 bp, synthetic construct
  source 1..269: mol_type = other DNA
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 7 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 8 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 9 - DNA, 89 bp, synthetic construct
  source 1..89: mol_type = other DNA
gcggccaattcagtcgataactataacggtcctaaggtagcgatttaaatcccgccatgctacttatctacgtagccatgctctaggta

SEQ ID NO: 10 - DNA, 30 bp, synthetic construct
  source 1..30: mol_type = other DNA
tctagcggccgcgaattcgatatcaagctt

SEQ ID NO: 11 - DNA, 21 bp, synthetic construct
  source 1..21: mol_type = other DNA
ggcggccgcgcaccgtcgact

SEQ ID NO: 12 - DNA, 79 bp, synthetic construct
  source 1..79: mol_type = other DNA
ctcgactagagcatggctacgtagataagtagcatggcgggatttaaattagggataacagggtaatggcgcgggccgc

SEQ ID NO: 13 - DNA, 3288 bp, synthetic construct
  source 1..3288: mol_type = other DNA
ggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgcaattgtatctaagccgtgtaatgagaacatccacaccttagtgaatcgatgcgccgcttcggaataccgttttggctacctgttactaagcccatcgcgattttcaggtatcgtgcacgtagggttgcaccgcacgcatgtcgaactggtggcgaagtacgattccacgaccggtgtacgattcaactacgcggacgtgacgagcttcttatatatgcttcgcccgccggaccggcctcgtgatggggtagctgcgcatgagcttatgacaaataacgagtgtgtacttgtttaatcatctcacagtgaaagtcgggagaataggagccgctacacacattttaccgcaactacacctaactgagatactaccatagatgactacccatccctctaggccttagataaccggatacagtgactttgataggtttgtggggtacagctatgacttgcatagctgcgtatggaggaaggaacttttgcgtgttagtatgttgacccgtgtattacgcatgcgggtagatcatgtgggtagagacatccaggtcaagttctcgaccttctcgtgggaggtgatccagttcactataggaccattccattcgagcatggcactaagtacgccgtccccattctggtaatcatcatccctatcagggtttggagtgagtggtgacggttattccccaggaatggacttcttattcacagtcggtcacattgggctactccttgggtcttccgcttggcccggtctgttgggccgccattgcgtgagtttcggccctgtgctgcgctgtgtcggcgattctcattggggcctcacatctggaaaccccgacctattttgacggcaccattggcggaagttgctgggcttgtgcaccgtgaagtcctccgcgggcctcccccctacaaaagacgataagctccggcaagcagcattgatcaacgcaaggatcggcgatattaacagagatacggctgattactcttgttggtgtggtatcgctaaactgcgccgcggagccttatggcaaagtcgtccgcgaaccattccggtagcgcttaaggtccatagcacattcatcgcatcccggcgtgcgttcaatttgacgaccccttggcgcaaaggtgctggccacgtgctaaattaaagcggctgcactgctgtaaggtccgttacggagagggcggcctggggagagcactagcccatcgacctgtacaggaacactctatattgttctcggacggacagattactagagtgccgctttcagcccccctgtcgtcgccgacgtctgtaatatggcgttgttgtgattccactctattgaggcattaactgatgcgtaaggagatctggaatgaactggcctatgtcactgaaactgtgcaaacacccaatgtcgttagtgtaggttctgaccgatacctgcttcgttgagaactcacaattttacaactggggacataatccctacgcccatcatctactcgcgtccctgtggctccagttcatgtaatgggagagtatcctccacaagatctagtgctatggtggtatagtaagctcgtactgtggtagacgcgacacgggtaggaccatcagtaataaggatagtgcgaaagctcacagaccactgcctatagggggtgcttacttttacaaaaagcgactgtcagtataaccccacgaggattcgaaaaggtgaaccgacccagacgatccggaggcacgggcctcaaagccgcgacacgacggctgtcggccggtaacagtaaccccggagtgaactcctatggggctggatagaacagccctggtgggccccatcagcaacccgaatacgtggcttttcgggaggcggccggaggggcgatgtcttccactattcgaggccgttcgttaatacttgttgcgttcctagccgctatatttgtctctttgccgactaatgtggacaagcacaccatagccatttatcggagcgcctcggaatacggtatgagcaggcgcctcgtgagaccattgcgaataccaggtgtcctgtggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagt

SEQ ID NO: 14 - DNA, 150 bp, synthetic construct
  source 1..150: mol_type = other DNA
taactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 15 - DNA, 11 bp, synthetic construct
  source 1..11: mol_type = other DNA
catgtacatgt

SEQ ID NO: 16 - DNA, 816 bp, synthetic construct
  source 1..816: mol_type = other DNA
atgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 17 - DNA, 668 bp, synthetic construct
  source 1..668: mol_type = other DNA
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca